(19) United States
(12) Patent Application Publication
(10) Pub. No.: US 2017/0059582 A1
(43) Pub. Date: Mar. 2, 2017

(54) METHODS FOR DIAGNOSING CELIAC DISEASE USING CIRCULATING CYTOKINES/CHEMOKINES

(71) Applicant: ImmusanT, Inc., Cambridge, MA (US)

(72) Inventor: Robert P. Anderson, Shrewsbury, MA (US)

(73) Assignee: ImmusanT, Inc., Cambridge, MA (US)

(21) Appl. No.: 15/306,126

(22) PCT Filed: Apr. 24, 2015
(86) PCT No.: PCT/US2015/027522

§ 371 Date: Oct. 24, 2016

Related U.S. Application Data
(60) Provisional application No. 62/014,666, filed on Jun. 19, 2014.
(60) Provisional application No. 62/043,395, filed on Aug. 28, 2014.
(60) Provisional application No. 62/043,390, filed on Aug. 28, 2014.
(60) Provisional application No. 62/043,386, filed on Aug. 28, 2014.
(60) Provisional application No. 62/057,163, filed on Sep. 29, 2014.
(60) Provisional application No. 62/057,152, filed on Sep. 29, 2014.
(60) Provisional application No. 62/082,832, filed on Nov. 21, 2014.
(60) Provisional application No. 62/116,027, filed on Feb. 13, 2015.
(60) Provisional application No. 62/115,963, filed on Feb. 13, 2015.
(60) Provisional application No. 62/116,002, filed on Feb. 13, 2015.
(60) Provisional application No. 62/116,052, filed on Feb. 13, 2015.
(60) Provisional application No. 62/115,897, filed on Feb. 13, 2015.
(60) Provisional application No. 62/115,925, filed on Feb. 13, 2015.
(60) Provisional application No. 62/014,676, filed on Jun. 19, 2014.
(60) Provisional application No. 62/014,681, filed on Jun. 19, 2014.
(60) Provisional application No. 62/014,373, filed on Jun. 19, 2014.
(60) Provisional application No. 62/014,401, filed on Jun. 19, 2014.
(60) Provisional application No. 62/011,794, filed on Jun. 13, 2014.
(60) Provisional application No. 62/011,493, filed on Jun. 12, 2014.
(60) Provisional application No. 62/011,540, filed on Jun. 12, 2014.
(60) Provisional application No. 62/011,508, filed on Jun. 12, 2014.
(60) Provisional application No. 62/011,561, filed on Jun. 12, 2014.
(60) Provisional application No. 62/011,566, filed on Jun. 12, 2014.
(60) Provisional application No. 62/009,090, filed on Jun. 6, 2014.
(60) Provisional application No. 62/009,146, filed on Jun. 6, 2014.
(60) Provisional application No. 61/984,043, filed on Apr. 25, 2014.
(60) Provisional application No. 61/983,989, filed on Apr. 24, 2014.
(60) Provisional application No. 61/983,981, filed on Apr. 24, 2014.
(60) Provisional application No. 61/984,028, filed on Apr. 24, 2014.

(51) Int. Cl. G01N 33/68

(52) U.S. Cl. CPC G01N 33/6863; G01N 2800/06; G01N 2333/5421; G01N 2333/523; G01N 2800/52

(57) ABSTRACT

Provided herein are compositions, kits, and methods for measuring circulating cytokines and chemokines in a subject that has or is suspected of having Celiac disease.

### RELATED APPLICATIONS

This application claims the benefit under 35 U.S.C. §119(e) of U.S. provisional application No. 61/983,981, filed Apr. 24, 2014, U.S. provisional application No. 62/011,561, filed Jun. 12, 2014, U.S. provisional application No. 62/014,676, filed Jun. 19, 2014, U.S. provisional application No. 62/057,152, filed Sep. 29, 2014, U.S. provisional application No. 62/115,925, filed Feb. 13, 2015, U.S. provisional application No. 61/984,028, filed Apr. 24, 2014, U.S. provisional application No. 61/984,043, filed Apr. 25, 2014, U.S. provisional application No. 62/011,566, filed Jun. 12, 2014, U.S. provisional application No. 62/014,681, filed Jun. 19, 2014, U.S. provisional application No. 62/057,163, filed Sep. 29, 2014, U.S. provisional application No. 62/115,897, filed Feb. 13, 2015, U.S. provisional application No. 61/983,989, filed Apr. 24, 2014, U.S. provisional application No. 62/014,666, filed Jun. 19, 2014, U.S. provisional application No. 62/009,146, filed Jun. 6, 2014, U.S. provisional application No. 62/043,386, filed Aug. 28, 2014, U.S. provisional application No. 62/115,963, filed Feb. 13, 2015, U.S. provisional application No. 61/983,993, filed Apr. 24, 2014, U.S. provisional application No. 62/011,508, filed Jun. 12, 2014, U.S. provisional application No. 62/116,052, filed Feb. 13, 2015, U.S. provisional application No. 62/043,395, filed Aug. 28, 2014, U.S. provisional application No. 62/082,832, filed Nov. 21, 2014, U.S. provisional application No. 62/009,090, filed Jun. 6, 2014, U.S. provisional application No. 62/014,373, filed Jun. 19, 2014, U.S. provisional application No. 62/043,390, filed Aug. 28, 2014, U.S. provisional application No. 62/116,002, filed Feb. 13, 2015, U.S. provisional application No. 62/011,493, filed Jun. 12, 2014, U.S. provisional application No. 62/011,794, filed Jun. 13, 2014, U.S. provisional application No. 62/014,401, filed Jun. 19, 2014, U.S. provisional application No. 62/116,027, filed Feb. 13, 2015, and U.S. provisional application No. 62/011,540, filed Jun. 12, 2014, the contents of each of which are incorporated by reference herein in their entirety.

### BACKGROUND OF THE INVENTION

Celiac disease is an autoimmune-like disorder of the small intestine that occurs in people of all ages and affects approximately 1% of people in Europe and North America. Celiac disease causes damage to the villi of the small intestine due to an inappropriate immune response to gluten peptides, leading to malabsorption and an increased risk of intestinal cancer. Correctly diagnosing celiac disease is important in order to ensure that those affected by celiac disease receive proper treatment.

### SUMMARY OF THE INVENTION

The disclosure relates, at least in part, to the measurement of circulating cytokines and chemokines for use in identifying subjects having or suspected of having Celiac disease.

Aspects of the disclosure relate to a method, comprising measuring a level of at least one circulating cytokine or chemokine in a subject that has or is suspected of having celiac disease, wherein the subject has been administered a first composition comprising at least one gluten peptide, and assessing the likelihood the subject has celiac disease.

In some embodiments, the method further comprises obtaining a sample from the subject and the measuring is performed on the sample. In some embodiments, the sample from the subject is obtained 1 hour to 6 hours after the subject has been administered the first composition. In some embodiments, the sample from the subject is obtained 4 hours to 6 hours after the subject has been administered the first composition. In some embodiments, the sample from the subject is a plasma, serum or urine sample. In some embodiments, the subject has been administered the first composition by injection. In some embodiments, the method further comprises administering the first composition to the subject prior to measuring the level of the at least one circulating cytokine or chemokine. In some embodiments, the at least one circulating cytokine or chemokine is MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 or sCD40L. In some embodiments, the at least one circulating cytokine or chemokine is MCP-1, IL-6, IL-8 or G-CSF. In some embodiments, the at least one circulating cytokine or chemokine is MCP-1. In some embodiments, the at least one circulating cytokine or chemokine is IL-2. In some embodiments, the at least one circulating cytokine or chemokine is IL-2, IL-8, or MCP-1. In some embodiments, the at least one circulating cytokine or chemokine is at least four circulating cytokines or chemokines comprising IL-2, IL-8, and MCP-1. In some embodiments, the at least one circulating cytokine or chemokine is at least two circulating cytokines or chemokines comprising IL-8 and MCP-1. In some embodiments, the at least one circulating cytokine or chemokine is selected from MCP-1, IL-10, IL-6, IL-8, IL-2 and G-CSF. In some embodiments, the at least one circulating cytokine or chemokine is MCP-1. In some embodiments, the at least one circulating cytokine or chemokine is IL-2. In some embodiments, the at least one circulating cytokine or chemokine is IL-2, IL-8, IL-10, or MCP-1. In some embodiments, the at least one circulating cytokine or chemokine is at least three circulating cytokines or chemokines comprising IL-2, IL-8, and MCP-1. In some embodiments, the at least one circulating cytokine or chemokine is at least two circulating cytokines or chemokines comprising IL-8 and MCP-1.

In some embodiments, an elevated level of the at least one circulating cytokine or chemokine compared to a control level of the at least one circulating cytokine or chemokine indicates that the subject has celiac disease, and the step of assessing comprises comparing the level of the at least one circulating cytokine or chemokine to a control level of the at least one circulating cytokine or chemokine. In some embodiments, the control level is a baseline level. In some embodiments, the baseline level is a level of the at least one circulating cytokine or chemokine prior to administration of the first composition.

In some embodiments, the method further comprises recording whether or not the subject has celiac disease based on the assessing. In some embodiments, the method further comprises treating, suggesting a treatment, or giving information in regard to a treatment to the subject. In some embodiments, the treating or treatment comprises administration of a second composition comprising a gluten peptide to the subject. In some embodiments, measuring the level of the at least one circulating cytokine or chemokine comprises an immuno-based assay. In some embodiments, the immuno-based assay comprises an ELISA or a multiplex bead-based assay.

In some embodiments, the first composition comprises at least one of: (a) a first peptide comprising the amino acid sequence PFPQPELPY (SEQ ID NO: 1) and PQPELPYPQ (SEQ ID NO: 2); (b) a second peptide comprising the amino acid sequence PFPQPEQPF (SEQ ID NO: 3) and PQPEQPFPW (SEQ ID NO: 4); and (c) a third peptide comprising the amino acid sequence PIPEQPQPY (SEQ ID NO: 5). In some embodiments, the first composition comprises the first and second peptide, the first and third peptide, or the second and third peptide. In some embodiments, the first composition comprises the first and second peptide. In some embodiments, the first composition comprises the first, second, and third peptide. In some embodiments, the first peptide comprises LQPFPQPELPYPQPQ (SEQ ID NO: 6); the second peptide comprises QPFPQPEQPFPWQP (SEQ ID NO: 7); and the third peptide comprises PEQPIPEQPQPYPQQ (SEQ ID NO: 8). In some embodiments, the first, second and/or third peptides comprise an N-terminal acetyl group or pyroglutamate group, and/or a C terminal amide group. In some embodiments, the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and the third peptide comprises EPEQPIPEQPQPYPQQ (SEQ ID NO: 11), wherein the N-terminal E is a pyroglutamate. In some embodiments, the first composition comprises 10 micrograms of the first peptide and an equimolar amount of each of the second and third peptides; 15 micrograms of the first peptide and an equimolar amount of each of the second and third peptides; 20 micrograms of the first peptide and an equimolar amount of each of the second and third peptides; or 50 micrograms of the first peptide and an equimolar amount of each of the second and third peptides. In some embodiments, the first composition is administered once to the subject.

In some embodiments, the second composition comprises at least one of:
(a) the first peptide comprising the amino acid sequence PFPQPELPY (SEQ ID NO: 1) and PQPELPYPQ (SEQ ID NO: 2);
(b) the second peptide comprising the amino acid sequence PFPQPEQPF (SEQ ID NO: 3) and PQPEQPFPW (SEQ ID NO: 4); and
(c) the third peptide comprising the amino acid sequence PIPEQPQPY (SEQ ID NO: 5).

In some embodiments, the second composition comprises the first and second peptide, the first and third peptide, or the second and third peptide. In some embodiments, the second composition comprises the first and second peptide. In some embodiments, the second composition comprises the first, second, and third peptide. In some embodiments, the first peptide comprises LQPFPQPELPYPQPQ (SEQ ID NO: 6); the second peptide comprises QPFPQPEQPFPWQP (SEQ ID NO: 7); and the third peptide comprises PEQPIPEQPQPYPQQ (SEQ ID NO: 8). In some embodiments, the first, second and/or third peptides comprise an N-terminal acetyl group or pyroglutamate group, and/or a C terminal amide group. In some embodiments, the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and the third peptide comprises EPEQPIPEQPQPYPQQ (SEQ ID NO: 11), wherein the N-terminal E is a pyroglutamate.

In some embodiments, the subject is HLA-DQ2.5 positive.

In some embodiments, the method further comprises measuring a T cell response to the first composition comprising the at least one gluten peptide. In some embodiments, measuring a T cell response comprises contacting a sample comprising a T cell from the subject with the first composition comprising the at least one gluten peptide and measuring the T cell response in the sample. In some embodiments, the T cell response is measured by measuring a level of IFN-γ. In some embodiments, measuring the level of IFN-γ comprises an immuno-based assay. In some embodiments, the immuno-based assay comprises an ELISA or a multiplex bead-based assay.

Other aspects of the disclosure relate to kits. In some embodiments, the kit comprises i) the first composition as defined in any one of the embodiments above, ii) a means for injecting the first composition, and iii) a binding partner for the at least one cytokine or chemokine as defined in any one of the embodiments above. In some embodiments, the kit comprises i) the first composition as defined in any one of the embodiments above and ii) a binding partner for MCP-1. In some embodiments, the kit further comprises a binding partner for IL-2, a binding partner for IL-8, and/or a binding partner for IL-10. In some embodiments, the kit further comprises a binding partner for IFN-γ. In some embodiments, the kit further comprises the second composition as defined in any one of the embodiments above.

In any one of the methods, compositions or kits provided herein, the composition comprising at least one gluten peptide, such as the first composition, is a composition comprising a protein that comprises the at least one gluten peptide. In any one of the methods, compositions or kits provided herein the composition comprising at least one gluten peptide, such as the first composition, is administered orally. In any one of the methods, compositions or kits provided herein, the composition comprising at least one gluten peptide, such as the first composition, is a composition comprising a protein that comprises the at least one gluten peptide and the composition is administered orally. In one embodiment of any one of these methods, compositions or kits, the composition comprising a protein that comprises the at least one gluten peptide is a composition comprising gluten, such as a foodstuff that contains gluten. For example, the composition comprising a gluten peptide may be a foodstuff (e.g., a baked good such a cookie, muffin, or bread) containing wheat gluten, barley hordein, and/or rye secalin. In some embodiments, the foodstuff contains 3 grams of gluten. In some embodiments, the composition is administered orally once to the subject.

In some embodiments of any one of the methods described above, the subject has been on a gluten-free diet for a defined period of time or has not been on a gluten free diet prior to performance of the method. In some embodiments, the subject has been on a gluten-free diet for no more than 14 days, no more than 30 days, no more than 60 days, or no more than 90 days. In some embodiments, the subject has been on a gluten-free diet for 14 days, 30 days, 60 days, or 90 days. In some embodiments, the subject has not been on a gluten-free diet prior to performance of any one of the methods described herein.

In some embodiments of any one of the methods described herein, the subject is a subject who has been subjected to or is subjected to a gluten challenge, e.g., by administering a composition comprising a gluten protein or any composition described herein. In some embodiments of any one of the methods described herein, the subject is a subject who has been administered a composition comprising a gluten protein or any composition described herein.

The disclosure also relates, in part, to circulating cytokines and chemokines and T cells for use in assessing tolerance or efficacy of treatment of Celiac disease.

Aspects of the disclosure relate to a method for assessing tolerance to gluten or a gluten peptide in a subject having or suspected of having Celiac disease, the method comprising: (a) measuring in a subject having or suspected of having Celiac disease that has been administered a first composition comprising at least one gluten peptide a level of at least one circulating cytokine or chemokine; and (b) assessing the tolerance of the subject to the at least one gluten peptide based on the measuring.

Other aspects of the disclosure relate to a method for assessing tolerance to gluten or a gluten peptide in a subject having or suspected of having Celiac disease, the method comprising: (a) measuring T cell response in a subject having or suspected of having Celiac disease that has been administered a first composition comprising at least one gluten peptide to the at least one gluten peptide of the first composition; and (b) assessing the tolerance of the subject to the at least one gluten peptide based on the measuring. The measuring of the T cell response may be performed alone or in combination with a measurement of the at least one circulating cytokine or chemokine.

Other aspects of the disclosure relate to a method for identifying a subject as being suitable for treatment with any of the compositions comprising a gluten peptide as provided herein. In one embodiment of such methods, the method comprises measuring in a subject having or suspected of having Celiac disease that has been administered the composition comprising at least one gluten peptide a level of at least one circulating cytokine or chemokine; and assessing the suitability of treating the subject with the composition. In another embodiment of such methods, the method comprises measuring in a subject having or suspected of having Celiac disease that has been administered the composition comprising a T cell response to the composition; and assessing the suitability of treating the subject with the composition.

In some embodiments of any one of the methods provided herein, the method further comprises obtaining a sample from the subject and the measuring is performed on the sample. In some embodiments, the subject has been administered the first composition by injection, such as intradermal injection, or oral administration. In some embodiments, the subject has been administered the first composition by injection, such as intradermal injection. In some embodiments, the method further comprises administering the first composition to the subject prior to the measuring.

In some embodiments of any one of the methods provided herein, the assessing comprises comparing the level of the at least one circulating cytokine or chemokine to a circulating cytokine or chemokine control level.

In some embodiments of any one of the methods provided herein, the method further comprises treating the subject prior to measuring and assessing. In some embodiments of any one of the methods provided herein, the method further comprises treating the subject or suggesting a treatment to the subject based on the assessing. In some embodiments of any one of the methods provided herein, the treating comprises continuing with the treatment, or the suggesting comprises suggesting the subject continue with the treatment, based on the assessing. In some embodiments of any one of the methods provided herein, the treating comprises ceasing the treatment, or the suggesting comprises suggesting the subject cease the treatment, based on the assessing. In some embodiments of any one of the methods provided herein, the treating comprises administering a different or additional treatment, or the suggesting comprises suggesting the subject be treated with an additional or different treatment, based on the assessing.

In some embodiments of any one of the methods provided herein, the method further comprises recording the level(s), the result(s) of the assessing and/or the treatment, or suggestion for treatment, based on the assessing.

In some embodiments of any one of the methods provided herein, the at least one circulating cytokine or chemokine is selected from MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 and sCD40L. In some embodiments, the at least one circulating cytokine or chemokine is selected from MCP-1, IL-10, IL-6, IL-8, IL-2 and G-CSF. In some embodiments, the at least one circulating cytokine or chemokine is MCP-1. In some embodiments, the at least one circulating cytokine or chemokine is IL-2. In some embodiments, the at least one circulating cytokine or chemokine is IL-2, IL-8, IL-10, or MCP-1. In some embodiments, the at least one circulating cytokine or chemokine is at least three circulating cytokines or chemokines comprising IL-2, IL-8, and MCP-1. In some embodiments, the at least one circulating cytokine or chemokine is at least two circulating cytokines or chemokines comprising IL-8 and MCP-1.

In some embodiments of any one of the methods provided herein, measuring the level of the at least one circulating cytokine or chemokine comprises an immuno-based assay. In some embodiments, the immuno-based assay comprises an ELISA or a multiplex bead-based assay. In some embodiments, the sample obtained from the subject is a plasma, serum, or urine sample. In some embodiments, the sample obtained from the subject is a plasma or serum sample. In some embodiments, the sample obtained from the subject is a urine sample.

In some embodiments of any one of the methods provided herein, the composition comprises at least one of:

(a) a first peptide comprising the amino acid sequence PFPQPELPY (SEQ ID NO: 1) and PQPELPYPQ (SEQ ID NO: 2);

(b) a second peptide comprising the amino acid sequence PFPQPEQPF (SEQ ID NO: 3) and PQPEQPFPW (SEQ ID NO: 4); and

(c) a third peptide comprising the amino acid sequence PIPEQPQPY (SEQ ID NO: 5). In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises LQPFPQPELPYPQPQ (SEQ ID NO: 6); the second peptide comprises QPFPQPEQPFPWQP (SEQ ID NO: 7); and the third peptide comprises PEQPIPEQPQPYPQQ (SEQ ID NO: 8). In some embodiments, the first, second and/or third peptides comprise an N-terminal acetyl group or pyroglutamate group, and/or a C terminal amide group. In some embodiments, the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and the third peptide comprises EPEQPIPEQPQPYPQQ (SEQ ID NO: 11), wherein the N-terminal E is a pyroglutamate.

In some embodiments of any one of the methods provided herein, the method further comprises orally administering or directing the subject to consume gluten. In some embodiments, the subject is orally administered or directed to consume gluten for three days. In some embodiments, a measuring step is performed six days after the last of the gluten is orally administered or consumed. In some embodiments, the measuring comprises determining a T cell response to the first composition comprising the at least one gluten peptide. In some embodiments, measuring a T cell response comprises contacting a sample comprising a T cell from the subject (e.g., a whole blood sample) with the first composition comprising the at least one gluten peptide and measuring the T cell response in the sample. In some embodiments, the T cell response is measured by measuring a level of IFN-γ. In some embodiments, measuring the level of IFN-γ comprises an immuno-based assay. In some embodiments, the immuno-based assay comprises an ELISA or a multiplex bead-based assay.

In some embodiments of any one of the methods provided herein, the method further comprises measuring a T cell response to the first composition comprising the at least one gluten peptide. In some embodiments, measuring a T cell response comprises contacting a sample comprising a T cell from the subject (e.g., a whole blood sample) with the first composition comprising the at least one gluten peptide and measuring the T cell response in the sample. In some embodiments, the T cell response is measured by measuring a level of IFN-γ. In some embodiments, measuring the level of IFN-γ comprises an immuno-based assay. In some embodiments, the immuno-based assay comprises an ELISA or a multiplex bead-based assay.

Other aspects of the disclosure relate to a method for assessing the efficacy of treatment of celiac disease, the method comprising (a) measuring in a subject that has been administered a first composition comprising at least one gluten peptide (i) a level of at least one circulating cytokine or chemokine, and/or (ii) a level of at least one circulating T cell; and (b) assessing the efficacy based on the measuring. In some embodiments, the method can further comprise (c) treating the subject, or suggesting a treatment to the subject, based on the assessing. In some embodiments, the method further comprises obtaining a sample from the subject and the measuring is performed on the sample. In some embodiments, the subject has been administered the first composition by injection, such as intradermal injection, or oral administration. In some embodiments, the subject has been administered the first composition by injection, such as intradermal injection. In some embodiments, the method further comprises administering the first composition to the subject prior to the measuring.

In some embodiments of any one of the methods provided herein, the assessing comprises comparing the level of the at least one circulating cytokine or chemokine and/or the level of at least one circulating T cell to a circulating cytokine or chemokine control level and/or a circulating T cell control level.

In some embodiments of any one of the methods provided herein, the treating comprises continuing with the treatment, or the suggesting comprises suggesting the subject continue with the treatment, based on the assessing. In some embodiments, the treating comprises ceasing the treatment, or the suggesting comprises suggesting the subject cease the treatment, based on the assessing. In some embodiments, the treating comprises administering a different or additional treatment, or the suggesting comprises suggesting the subject be treated with an additional or different treatment, based on the assessing.

In some embodiments of any one of the methods provided herein, the method further comprises recording the level(s), the result(s) of the assessing and/or the treatment, or suggestion for treatment, based on the assessing.

In some embodiments of any one of the methods provided herein, the at least one circulating cytokine or chemokine is selected from MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 and sCD40L. In some embodiments, the at least one circulating cytokine or chemokine is selected from MCP-1, IL-10, IL-6, IL-8, IL-2 and G-CSF. In some embodiments, the at least one circulating cytokine or chemokine is MCP-1. In some embodiments, the at least one circulating cytokine or chemokine is IL-2. In some embodiments, the at least one circulating cytokine or chemokine is IL-2, IL-8, IL-10, or MCP-1. In some embodiments, the at least one circulating cytokine or chemokine is at least three circulating cytokines or chemokines comprising IL-2, IL-8, and MCP-1. In some embodiments, the at least one circulating cytokine or chemokine is at least two circulating cytokines or chemokines comprising IL-8 and MCP-1. In some embodiments, measuring the level of the at least one circulating cytokine or chemokine comprises an immuno-based assay. In some embodiments, the immuno-based assay comprises an ELISA or a multiplex bead-based assay.

In some embodiments of any one of the methods provided herein, the at least one circulating T cell recognizes the at least one gluten peptide in the composition. In some embodiments, measuring the level of the at least one circulating T cell comprises a Major Histocompatibility Complex (MHC) tetramer assay.

In some embodiments of any one of the methods provided herein, the sample obtained from the subject is a plasma, serum, or urine sample. In some embodiments, the sample obtained from the subject is a plasma or serum sample. In some embodiments, the sample obtained from the subject is a urine sample. In some embodiments, the sample is obtained from the sample is obtained within 4 hours to 6 hours of administration of the composition.

In some embodiments of any one of the methods provided herein, the composition comprises at least one of:

(a) a first peptide comprising the amino acid sequence PFPQPELPY (SEQ ID NO: 1) and PQPELPYPQ (SEQ ID NO: 2);

(b) a second peptide comprising the amino acid sequence PFPQPEQPF (SEQ ID NO: 3) and PQPEQPFPW (SEQ ID NO: 4); and

(c) a third peptide comprising the amino acid sequence PIPEQPQPY (SEQ ID NO: 5). In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises LQPFPQPELPYPQPQ (SEQ ID NO: 6); the second peptide comprises QPFPQPEQPFPWQP (SEQ ID NO: 7); and the third peptide comprises PEQPIPEQPQPYPQQ (SEQ ID NO: 8). In some embodiments, the first, second and/or third peptides comprise an N-terminal acetyl group or pyroglutamate group, and/or a C terminal amide group. In some embodiments, the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and the third peptide comprises EPEQPIPEQPQPYPQQ (SEQ ID NO: 11), wherein the N-terminal E is a pyroglutamate. In some embodiments, the first composition comprises 10 micrograms of the first peptide and an equimolar amount of each of the second and third peptides; 15 micrograms of the first peptide and an equimolar amount of each of the second and third peptides; 20 micrograms of the first peptide and an equimolar amount of each of the second and third peptides; or 50 micrograms of the first peptide and an equimolar amount of each of the second and third peptides. In some embodiments, the first composition is administered once to the subject.

In some embodiments of any one of the methods provided herein, the method further comprises orally administering or directing the subject to consume gluten. In some embodiments, the subject is orally administered or directed to consume gluten for three days. In some embodiments, a measuring step is performed six days after the last of the gluten is orally administered or consumed. In some embodiments, the measuring comprises determining a T cell response to the first composition comprising the at least one gluten peptide. In some embodiments, measuring a T cell response comprises contacting a sample comprising a T cell from the subject (e.g., a whole blood sample) with the first composition comprising the at least one gluten peptide and measuring the T cell response in the sample. In some embodiments, the T cell response is measured by measuring a level of IFN-γ. In some embodiments, measuring the level of IFN-γ comprises an immuno-based assay. In some embodiments, the immuno-based assay comprises an ELISA or a multiplex bead-based assay.

In some embodiments of any one of the methods provided herein, the method further comprises measuring a T cell response to the first composition comprising the at least one gluten peptide. In some embodiments, the measuring a T cell response comprises contacting a sample comprising a T cell from the subject (e.g., a whole blood sample) with the first composition comprising the at least one gluten peptide and measuring the T cell response in the sample. In some embodiments, the T cell response is measured by measuring a level of IFN-γ. In some embodiments, measuring the level of IFN-γ comprises an immuno-based assay. In some embodiments, the immuno-based assay comprises an ELISA or multiplex bead-based assay.

Other aspects of the disclosure relate to kits. In some embodiments, the kit comprises i) the first composition as defined in any one of the embodiments herein, ii) a means for injecting the first composition, and iii) a binding partner for the at least one cytokine, chemokine, or T cell as defined in any one of the embodiments above. In some embodiments, the kit comprises i) the first composition as defined in any one of the embodiments herein and ii) a binding partner for MCP-1, IL-2 or IL-8. In some embodiments, when the kit comprises a binding partner for MCP-1, the kit further comprises a binding partner for IL-2, a binding partner for IL-8, or a binding partner for IL-10. In some embodiments, when the kit comprises a binding partner for MCP-1, the kit further comprises a binding partner for IL-2 and a binding partner for IL-8. In some embodiments of any one of the kits provided, the kit further comprises a binding partner for IFN-γ.

In any one of the methods, compositions or kits provided herein, the composition comprising at least one gluten peptide, such as the first composition, is a composition comprising a protein that comprises the at least one gluten peptide. In any one of the methods, compositions or kits provided herein, the composition comprising at least one gluten peptide, such as the first composition, is a composition comprising a protein that comprises the at least one gluten peptide and the composition is administered orally. In one embodiment of any one of these methods, compositions or kits, the composition comprising a protein that comprises the at least one gluten peptide is a composition comprising gluten, such as a foodstuff that contains gluten. For example, the composition comprising a gluten peptide may be a foodstuff (e.g., a baked good such a cookie, muffin, or bread) containing wheat gluten, barley hordein, and/or rye secalin. In some embodiments, the foodstuff contains 3 grams of gluten. In some embodiments, the composition is administered orally once to the subject.

In some embodiments of any one of the methods described above, the subject has been on a gluten-free diet for a defined period of time or has not been on a gluten free diet prior to performance of the method. In some embodiments, the subject has been on a gluten-free diet for no more than 14 days, no more than 30 days, no more than 60 days, or no more than 90 days. In some embodiments, the subject has been on a gluten-free diet for 14 days, 30 days, 60 days, or 90 days. In some embodiments, the subject has not been on a gluten-free diet prior to performance of any one of the methods described herein.

In some embodiments of any one of the methods described herein, the subject is a subject who has been subjected to or is subjected to a gluten challenge, e.g., by administering a composition comprising a gluten protein or any composition described herein. In some embodiments of any one of the methods described herein, the subject is a subject who has been administered a composition comprising a gluten protein or any composition described herein.

In some embodiments of any one of the methods described herein, the method further comprises recording a value (e.g., an amount or level) of one or more circulating cytokines or chemokines.

The details of one or more embodiments of the disclosure are set forth in the description below. Other features or advantages of the present disclosure will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-P are a series of graphs showing the mean fold-change in cytokine/chemokine plasma levels up to 6 hours after subjects were administered their initial dose of a gluten peptide composition or placebo (normal saline). All subjects were HLA-DQ2.5+, followed strict gluten free diet, and had celiac disease. Subjects in Cohort 1 and Cohort 2 were dosed 4 or 5 weeks following 3-day oral challenge with gluten 9 g/day. The gluten peptide composition was administered intradermally, 0.1 mL, to 8 subjects in Cohort 1 (150 μg; A, E, I, M), 10 in Cohort 2 (300 μg; B, F, J, N), and 7 in Cohort 7 (150 μg; C, G, K, O). Placebo (0.1 mL normal saline) was administered to 4 subjects in Cohort 1, 3 in Cohort 2 and 7 in Cohort 7 (D, H, L, P). Two-fold-increase in levels measured in triplicate plasma samples by Luminex 38-plex bead assay was considered significant.

FIGS. 2A-C are graphs of plasma levels of interleukin (IL)-2 (A), IL-8 (B), and monocyte chemotactic protein-1 (MCP-1), in individual subjects, which showed pronounced elevations in HLA-DQ2.5+ celiac disease patients on gluten free diet following administration. Cytokine and chemokine levels during the six hours after intradermal injection of the gluten peptide composition are expressed as fold-change compared to immediate pre-dose. Subjects shown from Cohort 1 are those who received 150 μg of the gluten peptide composition intradermally for 4-5 weeks after 3-day oral gluten challenge (n=8), subjects received 300 μg of the gluten peptide composition intradermally for 4-5 weeks after 3-day oral gluten challenge (n=10), and in Cohort 7 subjects were administered 150 μg intradermally without prior gluten challenge (n=7). Subjects in Cohorts 1, 2 and 7 who received placebo are not shown.

FIG. 3 is a diagram that shows the dosage regimen for administration in Example 1.

FIG. 4 is a table showing a representative subject from Cohort 1 who received 150 μg of the gluten peptide composition intradermally for 4-5 weeks after 3-day oral gluten challenge. The GI symptoms as well as the IL-2, IL-8, IL-10, and MCP-1 fold increase at visit 6 (first dose) and visit 21 (last dose).

FIGS. 5A-5U are a series of graphs showing the plasma levels of cytokines and chemokines in HLA-DQ2.5+ subjects administered the gluten peptide composition intradermally (150 ug in Cohorts 1 and 7, or 300 ug in Cohort 2) or placebo (0.1 mL normal saline) immediately after the 1st dose, or after the 16^{th} dose (the last dose after a regimen of twice a week for 8 weeks). The cytokines/chemokines tested included EGF, FGF-2, EOTAXIN, TGF-a, G-CSF, Flt-3L, GM-CSF, FRACKTALKINE, IFNa2, IFNg, GRO, IL-10, MCP-3, IL-12(P40), MDC, IL-12P70, IL-13, IL-15, sCD40L, IL-17A, IL-1RA, IL-1a, IL-9, IL-1b, IL-2, IL-3, IL-4, IL-5, IL-6, IL-7, IL-8, IP-10, MCP-1, MIP-1a, MIP-1b, TNFa, TNFb, and VEGF. FIGS. 5A-5D show the plasma levels of cytokines and chemokines 2 hours after the 1^{st} dose in Cohort 1, 2, 7, and placebo, respectively. FIGS. 5E-5H show the plasma levels of cytokines and chemokines 4 hours after the 1^{st} dose in Cohort 1, 2, 7, and placebo, respectively. FIGS. 5I-5L show the plasma levels of cytokines and chemokines 6 hours after the 1^{st} dose in Cohort 1, 2, 7, and placebo, respectively. FIGS. 5M-50 show the plasma levels of cytokines and chemokines 2 hours after the 16^{th} dose in Cohort 1, 7, and placebo, respectively. FIGS. 5P-5R show the plasma levels of cytokines and chemokines 4 hours after the 16^{th} dose in Cohort 1, 7, and placebo, respectively. FIGS. 5S-5U show the plasma levels of cytokines and chemokines 6 hours after the 16^{th} dose in Cohort 1, 7, and placebo, respectively.

FIG. 6 is a table showing responsiveness and tolerance by ex vivo whole blood cytokine release stimulated by immuno-dominant gluten-derived T cell epitopes before and after HLA-DQ2.5+ celiac disease patients were treated with the gluten peptide composition. Reactivity was defined by IFN-γ levels being greater than or equal to 7.2 pg/ml.

FIG. 7 is a diagram showing the study design described in Example 2.

FIG. 8 is a series of graphs of an IL-2 timecourse for plasma from subjects from cohort 1 (150 micrograms of peptide composition).

FIG. 9 is a series of graphs of an IL-8 timecourse for plasma from subjects from cohort 1 (150 micrograms of peptide composition).

FIG. 10 is a series of graphs of an MCP-1 timecourse for plasma from subjects from cohort 1 (150 micrograms of peptide composition).

FIG. 11 is a series of graphs of an IL-2 timecourse for plasma from subjects from cohort 2 (300 micrograms of peptide composition).

FIG. 12 is a series of graphs of an IL-8 timecourse for plasma from subjects from cohort 2 (300 micrograms of peptide composition).

FIG. 13 is a series of graphs of an MCP-1 timecourse for plasma from subjects from cohort 2 (300 micrograms of peptide composition).

FIG. 14 is a series of graphs of an IL-2 timecourse for plasma from subjects from cohort 7 (150 micrograms of peptide composition, biopsy).

FIG. 15 is a series of graphs of an IL-8 timecourse for plasma from subjects from cohort 7 (150 micrograms of peptide composition, biopsy).

FIG. 16 is a series of graphs of an MCP-1 timecourse for plasma from subjects from cohort 7 (150 micrograms of peptide composition, biopsy).

FIG. 17 is a series of graphs of an assessment of treatment at visits 6-21 (n=8), looking at fold-change versus pre-1st dose (V6) of IFN-γ, IL-2, IL-8, and MCP-1 in plasma samples from cohort 1 (150 micrograms of peptide composition).

FIGS. 18A-F are a series of graphs showing IL-8 and MCP-1 After 1st Dose of peptide composition 150 mcg intradermal (i.d.) or saline i.d. vs. oral cookie (3 g gluten) in DQ2.5+ Celiac Disease Subjects on a Gluten-free diet.

FIGS. 18G-L are a series of graphs showing IL-2 and IL-6 After 1st Dose of peptide composition 150 mcg i.d. or saline i.d. vs. oral cookie (3 g gluten) in DQ2.5+ Celiac Disease Subjects on a Gluten-free diet.

FIGS. 18M-R are a series of graphs showing IP-10 and IL-10 After 1st Dose of peptide composition 150 mcg i.d. or saline i.d. vs. oral cookie (3 g gluten) in DQ2.5+ Celiac Disease Subjects on a Gluten-free diet.

FIG. 19A is a schedule of assessments for Cohorts A-E as outlined in Example 4.

FIG. 19B is a schedule of assessments for Cohorts F and G as outlined in Example 4.

### DETAILED DESCRIPTION OF THE INVENTION

Celiac disease is diagnosed by small bowel biopsy showing villous atrophy, crypt hyperplasia and raised intra-epithelial lymphocytes, and supported by the presence of celiac disease-specific serology (IgA specific for transglutaminase and/or IgA and IgG specific for deamidated gliadin peptide). Intestinal histology and serological abnormalities normalize or improve within weeks to months of adopting gluten-free diet. In general, celiac disease can be excluded if certain alleles encoding HLA-DQA1*05, DQB1*02 and DQB1*0302 are not present. In patients who have adopted a gluten-free diet (GFD) without definitive diagnosis, reintroduction of gluten into the diet has been necessary to make a firm diagnosis of celiac disease. Reintroduction of >3 g/day gluten (about 1.5 slices of wheat bread) daily leads to intestinal tissue damage in the majority of patients with celiac disease usually strictly adherent to gluten free diet.

Small bowel biopsy typically requires an endoscopy, which is inconvenient and may be inconclusive if biopsies are not performed at multiple sites in the duodenum, processed meticulously and interpreted correctly. Requiring small bowel biopsy may also delay treatment because of the importance of continuing to consume gluten until after the procedure. Furthermore, celiac disease cannot be diagnosed in patients who have excluded gluten from their diet if serology and histology do show typical diagnostic features.

Oral gluten challenge for 3 days mobilizes gluten-reactive T cells that can generally be measured six days after commencing the challenge. However, patients may not tolerate consuming gluten for three days and results are not available for a number of days. As described herein, one or more levels of circulating cytokines and/or chemokines may be used to identify (e.g., diagnose) subjects as having Celiac disease after being administered one or more gluten peptides, such as by injection. The circulating cytokines and/or chemokines can then be measured soon after the administration. This allows for a diagnostic of Celiac disease that avoids the consumption of gluten for a number of days with results being more rapidly available. Accordingly, the disclosure provides compositions, methods, and kits related to measuring the level of at least one circulating cytokine and/or chemokine in subjects having or suspected of having Celiac disease as described herein.

Currently, the only approved treatment for celiac disease is a gluten-free diet. Other therapeutics are being developed, but assessing therapeutic efficacy has been challenging.

As described herein, one or more levels of circulating cytokines, chemokines, or T cells (or T cell response) may also be used to assess the therapeutic effectiveness of a celiac disease treatment, such as in restoring tolerance to gluten and allowing gluten to be safely included in the diet without causing disease relapse. Accordingly, the disclosure provides compositions, methods, and kits related to measuring the level of at least one circulating cytokine, chemokine, and/or T cell (or T cell response) in subjects having or suspected of having Celiac disease.

### Diagnostic Methods

One aspect of the disclosure relates to methods of identifying (e.g., diagnosing) subjects, such as subjects having or suspected of having Celiac disease.

In some embodiments, the method comprises measuring a level of at least one circulating cytokine or chemokine in a subject that has or is suspected of having celiac disease, wherein the subject has been administered a composition comprising at least one gluten peptide as described herein. In some embodiments, the method further comprises assessing the likelihood the subject has celiac disease. In some embodiments, assessing comprises comparing the level of the at least one circulating cytokine or chemokine to a control level of the at least one circulating cytokine or chemokine. Levels as used herein can be absolute or relative amounts. In some embodiments, assessing comprises determining the ratio of the level of the at least one circulating cytokine or chemokine to the control level. In some embodiments, the control level of the at least one circulating cytokine or chemokine is a baseline level of the circulating cytokine or chemokine. In some embodiments, the baseline level is the level of the circulating cytokine or chemokine in the subject prior to the administration of the one or more gluten peptides. In some embodiments of any one of the methods provided herein, the method can further comprise the step of determining a baseline level of the circulating cytokine or chemokine in the subject.

In some embodiments, an elevated level of the at least one circulating cytokine or chemokine compared to a control level, such as a baseline level, of the at least one circulating cytokine or chemokine indicates that the subject has or is likely to have celiac disease. In some embodiments, a ratio greater than 1 (e.g., greater than 2, 3, 4, 5, 6, 7, 8, 9, 10 or more) of the at least one circulating cytokine or chemokine to the control level, such as a baseline level, indicates that the subject has or is likely to have celiac disease. In some embodiments of any one of the methods provided herein, the method further comprises recording whether or not the subject has or is likely to have celiac disease based on the level or ratio.

In some embodiments, the level of the at least one circulating cytokine or chemokine is measured in a sample, e.g., a serum, plasma or urine sample, obtained from the subject. Samples are described elsewhere herein. In some embodiments, the sample is obtained from the subject within 1-24 hours, such as within 1-6 hours, of administration of the composition.

In some embodiments of any one of the methods provided herein, the method further comprises administering the composition comprising at least one gluten peptide as described herein to the subject, e.g., by injection. In some embodiments, the composition is administered via intradermal injection. In some embodiments, the composition is administered once. In some embodiments, the composition is administered once via intradermal injection.

In some embodiments of any one of the methods provided herein, the method further comprises performing other testing. Any method of other testing as described herein is contemplated. In some embodiments, the other testing comprises a serology test, genotyping, an intestinal biopsy, and/or a T cell response test. In some embodiments of any one of the methods provided herein, the method further comprises performing one or more additional tests on the subject. In some embodiments, the method further comprises contacting a sample comprising a T cell from the subject with a gluten peptide and measuring a T cell response in the sample. In some embodiments, a T cell response is measured by measuring a level of IFN-γ, where an increased level of IFN-γ compared to a control level (e.g., a level of IFN-γ in a sample that has not been contacted with a gluten peptide) may identify a subject as having Celiac disease. In some embodiments, a level of IFN-γ at or above a cut-off level (e.g., at or above 7.2 pg/ml) may identify a subject as having or likely as having Celiac disease.

In some embodiments of any one of the methods provided herein, the method further comprising treating or suggesting a treatment if the subject is identified as having or likely of having celiac disease. In some embodiments of any one of the methods provided herein, the method further comprises recommending a gluten-free diet and/or providing information in regard thereto to the subject. In some embodiments of any one of the methods provided herein, the method further comprises administering a treatment, or providing information in regard thereto, to the subject. Suitable treatments are described herein. In some embodiments, the treatment is a composition comprising a gluten peptide as described herein. In some embodiments, the treatment comprises a gluten-free diet.

### Therapeutic Efficacy Methods

One aspect of the disclosure relates to methods of assessing the efficacy of treatment of celiac disease (e.g., responsiveness to a therapeutic gluten peptide composition). In some embodiments, the method comprises (a) measuring in a subject that has been administered a first composition comprising at least one gluten peptide

(i) a level of at least one circulating cytokine or chemokine, and/or

(ii) a level of at least one circulating T cell; and (b) assessing the efficacy based on the measuring. The method, in some embodiments, can further include (c) treating the subject, or suggesting a treatment to the subject, based on the assessing.

In some embodiments, assessing comprises comparing the level of the at least one circulating cytokine, chemokine, or T cell to a control level of the at least one circulating cytokine, chemokine, or T cell. Levels as used herein can be absolute or relative amounts. In some embodiments, assessing comprises determining the ratio of the level of the at least one circulating cytokine, chemokine, or T cell to the control level. In some embodiments, the control level of the at least one circulating cytokine, chemokine, or T cell is a baseline level of the circulating cytokine, chemokine, or T cell. In some embodiments, the baseline level is the level of the circulating cytokine, chemokine, or T cell in the subject prior to the administration of the one or more gluten peptides. In some embodiments of any one of the methods provided herein, the method can further comprise the step of determining a baseline level of the circulating cytokine, chemokine, or T cell in the subject.

In some embodiments, the assessing comprises comparing the level of the at least one circulating cytokine or chemokine, and/or the level of at least one circulating T cell to a circulating cytokine or chemokine control level, such as a baseline level, and/or a circulating T cell control level, respectively. In some embodiments, the method further comprises recording the level(s), the result(s) of the assessing and/or the treatment, or suggestion for treatment, based on the assessing.

In some embodiments, a ratio of about 1 of the at least one circulating cytokine, chemokine, or T cell compared to a control level, such as a baseline level or negative control, of the at least one circulating cytokine, chemokine, or T cell indicates that a treatment has been effective. In some embodiments, a ratio of greater than 1 of the at least one circulating cytokine, chemokine, or T cell compared to a control level, such as a baseline level or negative control, of the at least one circulating cytokine, chemokine, or T cell indicates that a treatment has not been effective or completely effective. In some embodiments, a ratio of greater than or about equal to 1 of the at least one circulating cytokine, chemokine, or T cell compared to a control level, such as a positive control, of the at least one circulating cytokine, chemokine, or T cell indicates that a treatment has not been effective or completely effective. In some embodiments, a ratio of less than 1 of the at least one circulating cytokine, chemokine, or T cell compared to a control level, such as a positive control, of the at least one circulating cytokine, chemokine, or T cell indicates that a treatment has been effective. In some embodiments, the method further comprises recording whether or not the treatment has been effective or completely effective based on the level or ratio.

In some embodiments, a level of the at least one circulating cytokine, chemokine, or T cell that is no more than two-fold above a control level, such as a baseline level or negative control, of the at least one circulating cytokine, chemokine, or T cell indicates that a treatment has been effective. In some embodiments, a level of the at least one circulating cytokine, chemokine, or T cell that is two-fold or more above a control level, such as a baseline level or negative control, of the at least one circulating cytokine, chemokine, or T cell indicates that a treatment has not been effective or completely effective. In some embodiments, a level of IL-2 and IL-8 that are each no more than two-fold above a control level, such as a baseline level or negative control, of IL-2 and IL-8 indicates that a treatment has been effective. In some embodiments, a level of IL-2 and IL-8 that is two-fold or more above a control level, such as a baseline level or negative control, of IL-2 and IL-8 indicates that a treatment has not been effective or completely effective.

In some embodiments, the measuring is performed on a sample obtained from the subject, e.g., a serum, plasma or urine sample. Samples are described herein. In some embodiments, the method further comprises obtaining the sample from the subject. In some embodiments, the sample is obtained from the subject within 4-6 hours of administration of the composition. In some embodiments, the sample is obtained from the subject within 1-24 hours, such as within 1-6 hours, of administration of the composition.

In some embodiments, the method further comprises administering the composition comprising at least one gluten peptide as described herein to the subject, e.g., by injection or oral administration. In some embodiments, the composition is administered via intradermal injection. In some embodiments, the composition is administered once. In some embodiments, the composition is administered once via intradermal injection.

In some embodiments, treating comprises continuing with the treatment, or suggesting comprises suggesting the subject continue with the treatment, based on the assessing. In some embodiments, treating comprises ceasing the treatment, or suggesting comprises suggesting the subject cease the treatment, based on the assessing. In some embodiments, treating comprises administering a different or additional treatment, or the suggesting comprises suggesting the subject be treated with an additional or different treatment, based on the assessing. Exemplary treatments are described herein. In some embodiments, the treatment is a composition comprising a gluten peptide as described herein.

In some embodiments, the method further comprises orally administering or directing the subject to consume gluten prior to the measuring step. In some embodiments, the subject is orally administered or directed to consume gluten for at least three days. In some embodiments, the measuring step is performed six days after the last of the gluten is orally administered or consumed.

In some embodiments, the method further comprises performing other testing. Any method of other testing as described herein is contemplated. In some embodiments, the other testing comprises a serology test, genotyping, an intestinal biopsy, and/or a T-cell response test. In some embodiments, the method further comprises contacting a sample comprising a T cell from the subject (e.g., a whole blood sample) with a gluten peptide and measuring a T cell response in the sample. In some embodiments, a T cell response is measured by measuring a level of IFN-γ. In some embodiments, a decreased or similar level of IFN-γ compared to a control level (e.g., a level of IFN-γ in a sample that has not been contacted with a gluten peptide) indicates that a treatment has been effective. In some embodiments, a level of IFN-γ below a cut-off level (e.g., below 7.2 pg/ml) indicates that a treatment has been effective. In some embodiments, a T cell response is measured by measuring a level of IFN-γ, where an elevated level of IFN-γ compared to a control level (e.g., a level of IFN-γ in a sample that has not been contacted with a gluten peptide) indicates that a treatment has not been effective. In some embodiments, a level of IFN-γ at or above a cut-off level (e.g., at or above 7.2 pg/ml) indicates that a treatment has not been effective.

Another aspect of the disclosure relates to methods of assessing tolerance to a gluten peptide in a subject having Celiac disease. In some embodiments, tolerance is a state of lessened responsiveness or non-responsiveness of the immune system to a gluten peptide.

In some embodiments, the method can be any of the methods provided herein. In one embodiment, the method comprises (a) measuring in a subject that has been administered a first composition comprising at least one gluten peptide a level of at least one circulating cytokine or chemokine; and (b) assessing the tolerance of the subject to the at least one gluten peptide based on the measuring. In some embodiments, the subject is a subject that has previously received or is receiving treatment for Celiac disease. In some embodiments, the treatment is a composition comprising a gluten peptide as described herein.

In some embodiments, assessing comprises comparing the level of the at least one circulating cytokine or chemokine to a control level of the at least one circulating cytokine or chemokine. Levels as used herein can be absolute or relative amounts. In some embodiments, assessing comprises determining the ratio of the level of the at least one circulating cytokine or chemokine to the control level. In some embodiments, the control level of the at least one circulating cytokine or chemokine is a baseline level of the circulating cytokine or chemokine. In some embodiments, the baseline level is the level of the circulating cytokine or chemokine in the subject prior to the administration of the one or more gluten peptides. In some embodiments of any one of the methods provided herein, the method can further comprise the step of determining a baseline level of the circulating cytokine or chemokine in the subject.

In some embodiments, the assessing comprises comparing the level of the at least one circulating cytokine or chemokine to a circulating cytokine or chemokine control level, such as a baseline level. In some embodiments, the method further comprises recording the level(s) or the result(s) of the assessing.

In some embodiments, a ratio of about 2 or less (e.g., less than 2, less than 1, or less than 0.5) of the at least one circulating cytokine or chemokine compared to a control level, such as a baseline level or negative control, of the at least one circulating cytokine or chemokine indicates that the subject has been tolerized to the gluten peptide. In some embodiments, a ratio of greater than about 2 (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30) of the at least one circulating cytokine or chemokine to a control level, such as a baseline level or negative control, of the at least one circulating cytokine or chemokine indicates that the subject has not been tolerized to the gluten peptide. In some embodiments, the method further comprises recording whether or not the subject has been tolerized to a gluten peptide based on the level or ratio.

In some embodiments, the measuring is performed on a sample obtained from the subject, e.g., a serum, plasma, or urine sample. Samples are described herein. In some embodiments, the method further comprises obtaining the sample from the subject. In some embodiments, the sample is obtained from the subject within 1-24 hours, such as within 1-6 hours, of administration of the composition.

In some embodiments, the method further comprises administering the composition comprising at least one gluten peptide as described herein to the subject, e.g., by injection or oral administration. In some embodiments, the composition is administered via intradermal injection. In some embodiments, the composition is administered once. In some embodiments, the composition is administered once via intradermal injection.

In some embodiments, the method further comprises treating the subject or recommending a treatment to the subject based on the assessing. In some embodiments, treating comprises continuing with the treatment, or suggesting comprises suggesting the subject continue with the treatment, based on the assessing. In some embodiments, treating comprises ceasing the treatment, or suggesting comprises suggesting the subject cease the treatment, based on the assessing. In some embodiments, treating comprises administering a different or additional treatment, or the suggesting comprises suggesting the subject be treated with an additional or different treatment, based on the assessing. Exemplary treatments are described herein. In some embodiments, the treatment is a composition comprising a gluten peptide as described herein.

In some embodiments, the method further comprises orally administering or directing the subject to consume gluten prior to the measuring step. In some embodiments, the subject is orally administered or directed to consume gluten for at least three days. In some embodiments, the measuring step is performed six days after the gluten is orally administered or consumed.

In some embodiments, the method further comprises performing other testing. Any method of other testing as described herein is contemplated. In some embodiments, the other testing comprises a serology test, genotyping, an intestinal biopsy, and/or a T cell response test. In some embodiments, the method further comprises contacting a sample comprising a T cell from the subject (e.g., a whole blood sample) with a gluten peptide and measuring a T cell response in the sample. In some embodiments, a T cell response is measured by measuring a level of IFN-γ. In some embodiments, a decreased or similar level of IFN-γ compared to a control level (e.g., a level of IFN-γ in a sample that has not been contacted with a gluten peptide) may indicate that a subject has been tolerized to the gluten peptide. In some embodiments, a level of IFN-γ below a cut-off level (e.g., below 7.2 pg/ml) may indicate that a subject has been tolerized to the gluten peptide. In some embodiments, a T cell response is measured by measuring a level of IFN-γ, where an elevated level of IFN-γ compared to a control level (e.g., a level of IFN-γ in a sample that has not been contacted with a gluten peptide) may indicate that a subject has not been tolerized to the gluten peptide. In some embodiments, a level of IFN-γ at or above a cut-off level (e.g., above 7.2 pg/ml) may indicate that a subject has not been tolerized to the gluten peptide.

### Circulating Cytokines and Chemokines

Aspects of the disclosure relate to circulating cytokines and/or chemokines and uses thereof in a method, composition or kit described herein. As used herein, a “circulating cytokine or chemokine” is a cytokine or chemokine present in vivo in a subject, e.g., within the blood, plasma, serum, urine etc. of the subject, that may be measured in a sample obtained from the subject, e.g., in a blood (such as plasma or serum) or urine sample. The levels of such circulating cytokines or chemokines may be increased or decreased in the subject as a result of administration of a composition comprising a gluten peptide to the subject, such as for a treatment of Celiac disease. Non-limiting examples of circulating cytokines and chemokines that can be used in any one of the methods, compositions and kits described herein include, but are not limited to, those shown in Table 1. The sequences of the genes, mRNAs, and proteins for each cytokines/chemokine can be determined by one of ordinary skill in the art using the National Center for Biotechnology Information (NCBI) gene database at www.ncbi.nlm.nih.gov/gene.

TABLE 1
Cytokines and chemokines.

| Cytokine or | Cytokine or | | NCBI Reference |
|---|---|---|---|
| Chemokine | Chemokine Symbol | NCBI Human | Sequences |
| Symbol | (/Alternative Symbol) | Gene ID | Human Protein ID(s) |
| Chemokine (C-C | MCP-1/CCL2 | 6347 | NP_002973.1 |
| motif) ligand 2 | | | |
| Chemokine (C—X—C | IP-10/CXCL10 | 3627 | NP_001556.2 |
| motif) ligand | | | |
| 10 | | | |
| Interleukin 6 | IL-6 | 3569 | NP_000591.1 |
| Interleukin 8 | IL-8 | 3576 | NP_000575.1 |
| Granulocyte | G-CSF | 1440 | NP_000750.1, |
| colony- | | | NP_001171618.1, |
| stimulating factor | | | NP_757373.1, |
| | | | NP_757374.2 |
| Interleukin 2 | IL-2 | 3558 | NP_000577.2 |
| Interleukin 1 | IL-1RA | 3557 | NP_000568.1, |
| receptor | | | NP_776213.1, |
| antagonist | | | NP_776214.1, |
| | | | NP_776215.1 |
| Chemokine (C—X—C | GRO/CXCL1 | 2919 | NP_001502.1 |
| motif) ligand 1 | | | |
| Chemokine (C-C | EOTAXIN/CCL11 | 6356 | NP_002977.1 |
| motif) ligand 11 | | | |
| Granulocyte- | GM-CSF | 1437 | NP_000749.2 |
| macrophage | | | |
| colony- | | | |
| stimulating factor | | | |
| Interleukin 10 | IL-10 | 3586 | NP_000563.1 |
| Tumor necrosis | TNFa | 7124 | NP_000585.2 |
| factor alpha | | | |
| Interferon, alpha 2 | IFNa2 | 3440 | NP_000596.2 |
| Chemokine (C-C | MIP-1b/CCL4 | 6351 | NP_002975.1 |
| motif) ligand 4 | | | |
| Interleukin 12 | IL-12P70 (heterodimer | IL-12A 3592 | IL-12A |
| | of IL-12A and IL-12B) | IL-12B 3593 | NP_000873.2 |
| | | | IL-12B |
| | | | NP_002178.2 |
| Interleukin 1, | IL-1a | 3552 | NP_000566.3 |
| alpha | | | |
| Interleukin 17A | IL-17A | 3605 | NP_002181.1 |
| Epidermal growth | EGF | 1950 | NP_001171601.1, |
| factor | | | NP_001171602.1, |
| | | | NP_001954.2 |
| Chemokine (C-C | MIP-1a/CCL3 | 6348 | NP_002974.1 |
| motif) ligand 3 | | | |
| Chemokine (C—X3—C | FRACTALKINE/ | 6376 | NP_002987.1 |
| motif) | CX3CL1 | | |
| ligand 1 | | | |
| Interferon gamma | IFNg or IFN-γ | 3458 | NP_000610.2 |
| Vascular | VEGF | 7422 | NP_001020537.2, |
| endothelial | | | NP_001020538.2, |
| growth factor | | | NP_001020539.2, |
| | | | NP_001020540.2, |
| | | | NP_001020541.2, |
| | | | NP_001028928.1, |
| | | | NP_001165093.1, |
| | | | NP_001165094.1, |
| | | | NP_001165095.1, |
| | | | NP_001165096.1, |
| | | | NP_001165097.1, |
| | | | NP_001165098.1, |
| | | | NP_001165099.1, |
| | | | NP_001165100.1, |
| | | | NP_001165101.1, |
| | | | NP_001191313.1, |
| | | | NP_001191314.1, |
| | | | NP_001273973.1, |
| | | | NP_003367.4 |
| Interleukin 9 | IL-9 | 3578 | NP_000581.1 |
| Fibroblast growth | FGF-2 | 2247 | NP_001997.5 |
| factor 2 | | | |
| Interleukin 1, beta | IL-1b | 3553 | NP_000567.1 |
| Fms-related | Flt-3L | 2323 | NP_001191431.1, |
| tyrosine kinase 3 | | | NP_001191432.1, |
| ligand | | | NP_001265566.1, |
| | | | NP_001265567.1 |
| Interleukin 15 | IL-15 | 3600 | NP_000576.1, |
| | | | NP_751915.1 |
| Lymphotoxin | TNFb/LTA | 4049 | NP_000586.2, |
| alpha | | | NP_001153212.1 |
| Interleukin 12B | IL-12(P40)/IL12B | 3593 | NP_002178.2 |
| Chemokine (C-C | MCP-3/CCL7 | 6354 | NP_006264.2 |
| motif) ligand 7 | | | |
| Interleukin 4 | IL-4 | 3565 | NP_000580.1, |
| | | | NP_758858.1 |
| Chemokine (C-C | MDC/CCL22 | 6367 | NP_002981.2 |
| motif) ligand 22 | | | |
| Interleukin 13 | IL-13 | 3596 | NP_002179.2 |
| soluble CD40 | sCD40L | 959 | NP_000065.1 |
| ligand | | | |
| Transforming | TGF-a | 7039 | NP_001093161.1, |
| growth factor, | | | NP_003227.1 |
| alpha | | | |
| Interleukin 3 | IL-3 | 3562 | NP_000579.2 |
| Interleukin 5 | IL-5 | 3567 | NP_000870.1 |
| Interleukin 7 | IL-7 | 3574 | NP_000871.1, |
| | | | NP_001186815.1, |
| | | | NP_001186816.1, |
| | | | NP_001186817.1 |

In some embodiments, the at least one circulating cytokine or chemokine is MCP-1, IL-6, IL-10, IL-8, or G-CSF. In some embodiments, the at least one circulating cytokine or chemokine is IL-2, IL-8, IL-10, or MCP-1. In some embodiments, the at least one circulating cytokine or chemokine comprises one or more of IL-2, IL-8, IL-10, and MCP-1. In some embodiments, the at least one circulating cytokine or chemokine comprises IL-2, IL-8, IL-10, and MCP-1. In some embodiments, the at least one circulating cytokine or chemokine comprises IL-8, IL-10, and MCP-1. In some embodiments, the at least one circulating cytokine or chemokine comprises IL-2, IL-10, and MCP-1. In some embodiments, the at least one circulating cytokine or chemokine comprises IL-2, IL-8, and MCP-1. In some embodiments, the at least one circulating cytokine or chemokine comprises IL-2, IL-8, and IL-10. In some embodiments, the at least one circulating cytokine or chemokine is MCP-1, IL-6, IL-8, or G-CSF. In some embodiments, the at least one circulating cytokine or chemokine is IL-2, IL-8, or MCP-1. In some embodiments, the at least one circulating cytokine or chemokine comprises one or more of IL-2, IL-8, and MCP-1. In some embodiments, the at least one circulating cytokine or chemokine comprises IL-8 and MCP-1. In some embodiments, the at least one circulating cytokine or chemokine comprises IL-2 and MCP-1. In some embodiments, the at least one circulating cytokine or chemokine comprises IL-2 and IL-8. In some embodiments, the at least one circulating cytokine or chemokine comprises IL-2. In some embodiments, the at least one circulating cytokine or chemokine comprises IL-8. In some embodiments, the at least one circulating cytokine or chemokine comprises MCP-1. In some embodiments, the at least one circulating cytokine or chemokine comprises one or more of IL-2, IP-10, and IFN-γ. In some embodiments, the at least one circulating cytokine or chemokine comprises IL-2, IP-10, and IFN-γ.

In some embodiments, an elevated level (e.g., an elevated level of protein or nucleic acid (e.g., mRNA level)) of the at least one circulating cytokine or chemokine compared to a control level of the at least one circulating cytokine or chemokine indicates that the subject has or is likely to have celiac disease. In some embodiments, methods provided herein comprise use of the ratio of the level of the at least one circulating cytokine or chemokine to a control level, such as a baseline level.

In some embodiments, the level of more than one circulating cytokine or chemokine is measured, e.g., the level of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more circulating cytokines or chemokines.

Assays for detecting cytokine or chemokine protein levels include, but are not limited to, immunoassays (also referred to herein as immune-based or immuno-based assays, e.g., Western blot or enzyme-linked immunosorbent assay (ELISA)), Mass spectrometry, and multiplex bead-based assays. Binding partners for protein detection can be designed using methods known in the art and as described herein. In some embodiments, the protein binding partners, e.g., antibodies, bind to a part of or an entire amino acid sequence of at least one cytokine or chemokine, the sequence(s) being identifiable using the Genbank IDs described herein. Other examples of protein detection and quantitation methods include multiplexed immunoassays as described for example in U.S. Pat. Nos. 6,939,720 and 8,148,171, and published U.S. Patent Application No. 2008/0255766, and protein microarrays as described for example in published U.S. Patent Application No. 2009/0088329.

An exemplary ELISA involves at least one binding partner, e.g., an antibody or antigen-binding fragment thereof, with specificity for the at least one cytokine or chemokine.

The sample with an unknown amount of the at least one cytokine or chemokine can be immobilized on a solid support (e.g., a polystyrene microtiter plate) either non-specifically (via adsorption to the surface) or specifically (via capture by another binding partner specific to the same at least one cytokine, as in a “sandwich” ELISA). After the cytokine or chemokine is immobilized, the binding partner for the at least one cytokine or chemokine can be added, forming a complex with the immobilized at least one cytokine or chemokine. The binding partner can be attached to a detectable label as described herein (e.g., a fluorophore or an enzyme), or can itself be detected by an agent that recognizes the at least one cytokine or chemokine binding partner that is attached to a detectable label as described herein (e.g., a fluorophore or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

Assays may also include a multiplex bead-based assay, such as an assay commercially available from Luminex (see, e.g., the MAGPIX® system). Multiplex bead-based assays are known in the art.

Assays for detecting cytokine or chemokine nucleic acid, such as RNA, include, but are not limited to, Northern blot analysis, RT-PCR, sequencing technology, RNA in situ hybridization (using e.g., DNA or RNA probes to hybridize RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer with addressable location, such as Affymetrix microarray (Affymetrix®, Santa Clara, Calif.)). Designing nucleic acid binding partners, such as probes, is well known in the art. In some embodiments, the nucleic acid binding partners bind to a part of or an entire nucleic acid sequence of at least one cytokine or chemokine, the sequence(s) being identifiable using the Genbank IDs described herein.

### Circulating T Cells

Aspects of the disclosure relate to circulating T cells and uses thereof in a method or kit described herein. As used herein, a “circulating T cell” is a T cell present in vivo in a subject, e.g., within the blood of the subject, that may be measured in a sample obtained from the subject, e.g., in a blood (such as plasma or serum) sample. The levels of such circulating T cells may be increased or decreased in the subject as a result of administration of a composition comprising a gluten peptide to the subject. Non-limiting examples of circulating T cells that can be used in the methods and kits described herein include, but are not limited to, at least one circulating T cell that recognizes at least one gluten peptide, e.g., a gluten peptide comprised in a composition described herein. In some embodiments, the T cells recognizes at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). A T cell that recognizes a gluten peptide is a T cell that comprises a T cell receptor that binds to the gluten peptide and/or that binds to the gluten peptide attached to one or more Major Histocompatibility Complex (MHC) molecules. In some embodiments, the circulating T cell is a CD4⁺ T cell. In some embodiments, the level of more than one circulating T cell is measured. The circulating T cell may be measured by direct assessment of T cells, for example by staining with MHC-peptide multimer and flow cytometery or by functional cytokine release assays, such as interferon-γ secretion in plasma from whole blood incubated with the cognate peptide of the T cell population of interest (e.g., a gluten peptide described herein) or another T cell response method described herein or otherwise known in the art.

Assays for detecting circulating T cells include, but are not limited to, a Major Histocompatibility Complex (MHC) tetramer assay and a T cell response assay. Such assays are known in the art (see, e.g., John D. Altman et al. (1996). “Phenotypic Analysis of Antigen-Specific T Lymphocytes.” Science 274 (5284): 94-96; Hanne Quarsten et al. (2001) “Staining of Celiac Disease-Relevant T Cells by Peptide-DQ2 Multimers.” Journal of Immunology 167(9):4861-4868; Melinda Rai et al. (2007) “Tetramer visualization of gut-homing gluten-specific T cells in the peripheral blood of celiac disease patients.” PNAS 104(8): 2831-2836). T cell response assays are described herein and are known in the art (see, e.g., Ontiveros N, Tye-Din J A, Hardy M Y, Anderson R P. Ex vivo whole blood secretion of interferon-γ (IFN-γ) and IFN-γ-inducible protein-10 (IP-10) measured by ELISA are as sensitive as IFN-γ ELISpot for the detection of gluten-reactive T cells in HLA-DQ2.5+ associated celiac disease. Clin Exp Immunol. 2014; 175:305-315).

An exemplary MHC tetramer assay involves use of DQ2 (DQA1*0501/DQB1*0201) MHC molecules containing a biotin. The DQ2 molecules are mixed with peptides, e.g., gluten peptides, to form DQ2-peptide complexes. Tetramers may be made by conjugating the DQ2-peptide complexes with streptavidin labeled with a fluorophore. For tetramer staining, circulating T cells are contacted with the tetramers and the tetramers bound to the circulating T cells are then detected, e.g., by flow cytometry. Secondary T cell markers may also be used in connection with the tetramer assay, e.g., anti-CD4 antibodies, anti-CD3 antibodies, and anti-CD45RA antibodies.

### Samples

Samples, as used herein, refer to biological samples taken or derived from a subject, e.g., a subject having or suspected of having Celiac disease. Examples of samples include fluid samples. In some embodiments, the sample comprises plasma, serum or urine. In some embodiments of any one of the methods provided herein, the methods comprise obtaining or providing the sample. In some embodiments of any one of the methods provided herein, the sample is obtained from the subject after administration to the subject of a composition comprising a gluten peptide as described herein. In some embodiments, the sample is obtained, e.g., at least 1, 2, 3, 4, 5, or 6 hours after administration of the composition to the subject. In some embodiments, the sample is obtained, e.g., within 4 hours of administration of the composition. In some embodiments, the sample is obtained, e.g., 4 hours after administration of the composition. In some embodiments, the sample is obtained, e.g., within 24 hours or within 6 hours of administration of the composition. In some embodiments, the sample is obtained, e.g., within 1 hour to 24 hours, within 1 hour to 6 hours, within 2 hours to 6 hours, within 2 hours to 4 hours, within 3 hours to 5 hours, within 3 hours to 6 hours, within 4 hours to 6 hours, or within 5 hours to 6 hours of administration of the composition. In some embodiments, the sample is obtained within 4 hours to 6 hours of administration of the composition.

In some embodiments of any one of the methods provided herein, a second sample is obtained, e.g., a control sample. Controls and control samples are described herein. In some embodiments, the second sample is obtained prior to administration of a composition comprising a gluten peptide as described herein. In some embodiments, the second sample is obtained at least, e.g., 1, 2, 3, 4, 5, 6, 12, 24, or more hours before administration of the composition. In some embodiments, the second sample is obtained no more than 1, 2, 3, 4, 5, 6, 7, 8, 9, 10, 15, 20, 25, 30 or 60 minutes before administration of the composition. In some embodiments, the second sample is obtained at least, e.g., 1, 2, 3, 4, 5, 6, 12, 24, or more hours before administration of the composition. In some embodiments, additional samples are obtained, e.g., at different time points during treatment of a subject.

In some embodiments of any one of the methods provided herein, a sample is not contacted with a gluten peptide or a composition comprising a gluten peptide ex vivo after the sample is obtained from the subject.

### Subjects

A subject may include any subject that has or is suspected of having Celiac disease. Preferably, the subject is a human. In some embodiments, the subject has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). In some embodiments, the subject is HLA-DQ2.5 positive (i.e., has both susceptibility alleles DQA1*05 and DQB1*02). In some embodiments, a subject may have a family member that has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). The presence of susceptibility alleles can be detected by any nucleic acid detection method known in the art, e.g., by polymerase chain reaction (PCR) amplification of DNA extracted from the patient followed by hybridization with sequence-specific oligonucleotide probes. In some embodiments of any one of the methods provided herein, the subject is on a gluten-free diet. In some embodiments, the subject is a subject having been administered a treatment as described herein.

In some embodiments, the subject has been on a gluten-free diet for a defined period of time or has not been on a gluten free diet prior to performance of any one of the methods described herein. In some embodiments, the subject has been on a gluten-free diet for at least 14 days, at least 30 days, at least 60 days, or at least 90 days prior to performance of any one of the methods described herein. In some embodiments, the subject has been on a gluten-free for at least 14 days, at least 30 days, at least 60 days, or at least 90 days prior, but no more than 1 year. In some embodiments, the subject has been on a gluten-free diet for no more than 14 days, no more than 30 days, no more than 60 days, or no more than 90 days. In some embodiments, the subject has been on a gluten-free diet for 14 days, 30 days, 60 days, or 90 days. In some embodiments, the subject has not been on a gluten-free diet prior to performance of any one of the methods described herein. In some embodiments, a subject may be determined to have been on a gluten-free diet or not on a gluten-free diet by asking the subject whether they have ingested gluten during the defined period of time. In some embodiments, a subject may be determined to have been on a gluten-free diet or not on a gluten-free diet by performing other testing as described herein (e.g., biopsy or serology) or a T cell response assay as described herein.

### Controls and Control Subjects

In some embodiments, methods provided herein comprise measuring a level of at least one circulating cytokine or chemokine in a sample obtained from a subject after administration of a composition comprising a gluten peptide and comparing the level to one or more control levels. In some embodiments, the control level is the level of the circulating cytokine or chemokine prior to administration of the composition comprising a gluten peptide. In some embodiments, the control level is the level of the circulating cytokine or chemokine without the influence of the composition. In some embodiments, the control level is a baseline level.

In some embodiments, methods provided herein comprise measuring a level of a circulating cytokine or chemokine, or a circulating T cell in a sample obtained from a subject after administration of a composition comprising a gluten peptide and comparing the level to one or more control levels. In some embodiments, the control level is the level of the circulating cytokine or chemokine, or circulating T cell prior to administration of the composition comprising a gluten peptide. In some embodiments, the control level is the level of the circulating cytokine or chemokine, or circulating T cell without the influence of the composition. In some embodiments, the control level is a baseline level. In still other embodiments, the control level is a positive control level where the level is indicative of an immune response against a gluten peptide.

However, other or further controls are also contemplated. For example, a control level may be a level in a sample from a control subject (or subjects). In some embodiments, a control subject has one or more HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein but does not have Celiac disease. In some embodiments, a control subject does not have any of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein. In some embodiments, a control subject is a healthy individual not having or suspected of having Celiac disease. In some embodiments, a control level is a pre-determined value from a control subject or subjects, such that the control level need not be measured every time the methods described herein are performed.

### Gluten Peptides and Compositions Comprising Gluten Peptides

As used herein the term “gluten peptide” includes any peptide comprising a sequence derived from, or encompassed within, one or more of gluten proteins alpha (α), beta (β), γ (γ) and omega (ω) gliadins, and low and high molecular weight (LMW and HMW) glutenins in wheat, B, C and D hordeins in barley, β, γ and ω secalins in rye, and optionally avenins in oats, including deamidated variants thereof containing one or more glutamine to glutamate substitutions. In some embodiments, the gluten peptide(s) stimulate a CD4+ T cell specific response.

A gluten peptide may include one or more sequences of epitopes known to be recognized by a CD4⁺ T cell in a subject with Celiac disease, e.g., PELP (SEQ ID NO: 12), PELPY (SEQ ID NO: 13), QPELPYP (SEQ ID NO: 89), PQPELPY (SEQ ID NO: 90), FPQPELP, (SEQ ID NO: 91), PELPYPQ (SEQ ID NO: 92), FPQPELPYP (SEQ ID NO: 93), PYPQPELPY (SEQ ID NO:14), PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 5), EQPIPEQPQ (SEQ ID NO: 106), PQPELPYPQ (SEQ ID NO: 28), FRPEQPYPQ (SEQ ID NO: 29), PQQSFPEQQ (SEQ ID NO: 30), IQPEQPAQL (SEQ ID NO: 31), QQPEQPYPQ (SEQ ID NO: 32), SQPEQEFPQ (SEQ ID NO: 33), PQPEQEFPQ (SEQ ID NO: 34), QQPEQPFPQ (SEQ ID NO: 35), PQPEQPFCQ (SEQ ID NO: 36), QQPFPEQPQ (SEQ ID NO: 37), PFPQPEQPF (SEQ ID NO: 38), PQPEQPFPW (SEQ ID NO: 39), PFSEQEQPV (SEQ ID NO: 40), FSQQQESPF (SEQ ID NO: 41), PFPQPEQPF (SEQ ID NO: 42), PQPEQPFPQ (SEQ ID NO: 43), PIPEQPQPY (SEQ ID NO: 44), PFPQPEQPF (SEQ ID NO: 45), PQPEQPFPQ (SEQ ID NO: 46), PYPEQEEPF (SEQ ID NO: 47), PYPEQEQPF (SEQ ID NO: 48), PFSEQEQPV (SEQ ID NO: 49), EGSFQPSQE (SEQ ID NO: 50), EQPQQPFPQ (SEQ ID NO: 51), EQPQQPYPE (SEQ ID NO: 52), QQGYYPTSPQ (SEQ ID NO: 53), EGSFQPSQE (SEQ ID NO: 54), PQQSFPEQE (SEQ ID NO: 55), or QGYYPTSPQ (SEQ ID NO: 56) (see, e.g., Sollid L M, Qiao S W, Anderson R P, Gianfrani C, Koning F. Nomenclature and listing of celiac disease relevant gluten epitopes recognized by CD4⁺ T cells. Immunogenetics. 2012; 64:455-60; PCT Publication Nos.: WO/2001/025793, WO/2003/104273, WO/2005/105129, and WO/2010/060155). Preferably, in some embodiments, the gluten peptides that comprise sequences of epitopes such as those set forth in SEQ ID NO: 12, 13, etc., also comprise additional amino acids flanking either or both sides of the epitope. Preferably, in some embodiments, the gluten peptide(s) is/are at least 8 or 9 amino acids in length.

Exemplary gluten peptides and method for synthesizing or obtaining such peptides are known in the art (see, e.g., PCT Publication Nos.: WO/2001/025793, WO/2003/104273, WO/2005/105129, and WO/2010/060155, which are incorporated herein by reference in their entirety). A gluten peptide can be recombinantly and/or synthetically produced. In some embodiments, a gluten peptide is chemically synthesized, e.g., using a method known in the art. Non-limiting examples of peptide synthesis include liquid-phase synthesis and solid-phase synthesis. In some embodiments, a gluten peptide is produced by enzymatic digestion, e.g., by enzymatic digestion of a larger polypeptide into short peptides.

In some embodiments, one or more glutamate residues of a gluten peptide may be generated by tissue transglutaminase (tTG) deamidation activity upon one or more glutamine residues of the gluten peptide. This deamidation of glutamine to glutamate can cause the generation of gluten peptides that can bind to HLA-DQ2 or -DQ8 molecules with high affinity. This reaction may occur in vitro by contacting the gluten peptide composition with tTG outside of the subject or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, PFPQPQLPY (SEQ ID NO: 15) could become PFPQPELPY (SEQ ID NO: 1) after processing by tTG. Conservative substitution of E with D is also contemplated herein (e.g., PFPQPELPY (SEQ ID NO: 1) could become PFPQPDLPY (SEQ ID NO: 27). Exemplary peptides including an E to D substitution include peptide comprising or consisting of PFPQPDLPY (SEQ ID NO: 27), PQPDLPYPQ (SEQ ID NO: 94), PFPQPDQPF (SEQ ID NO: 95), PQPDQPFPW (SEQ ID NO: 96), PIPDQPQPY (SEQ ID NO: 97), LQPFPQPDLPYPQPQ (SEQ ID NO: 98), QPFPQPDQPFPWQP (SEQ ID NO: 99), or PQQPIPDQPQPYPQQ (SEQ ID NO: 100). Such substituted peptides can be the gluten peptides of any of the methods and compositions provided herein. Accordingly, gluten peptides that have not undergone deamidation are also contemplated herein (e.g., gluten peptides comprising or consisting of PQLP (SEQ ID NO: 16), PQLPY (SEQ ID NO: 17), QPQLPYP (SEQ ID NO: 101), PQPQLPY (SEQ ID NO: 102), FPQPQLP, (SEQ ID NO: 103), PQLPYPQ (SEQ ID NO: 104), FPQPQLPYP (SEQ ID NO: 105), PYPQPQLPY (SEQ ID NO: 18), PFPQPQLPY (SEQ ID NO: 19), PQPQLPYPQ (SEQ ID NO: 20), PFPQPQQPF (SEQ ID NO: 21), PQPQQPFPW (SEQ ID NO: 22), PIPQQPQPY (SEQ ID NO: 23), LQPFPQPQLPYPQPQ (SEQ ID NO: 24), QPFPQPQQPFPWQP (SEQ ID NO: 25), or PEQPIPQQPQPYPQQ (SEQ ID NO: 26), PQPQLPYPQ (SEQ ID NO: 57), FRPQQPYPQ (SEQ ID NO: 58), PQQSFPQQQ (SEQ ID NO: 59), IQPQQPAQL (SEQ ID NO: 60), QQPQQPYPQ (SEQ ID NO: 61), SQPQQQFPQ (SEQ ID NO: 62), PQPQQQFPQ (SEQ ID NO: 63), QQPQQPFPQ (SEQ ID NO: 64), PQPQQPFCQ (SEQ ID NO: 65), QQPFPQQPQ (SEQ ID NO: 66), PFPQPQQPF (SEQ ID NO: 67), PQPQQPFPW (SEQ ID NO: 68), PFSQQQQPV (SEQ ID NO: 69), FSQQQQSPF (SEQ ID NO: 70), PFPQPQQPF (SEQ ID NO: 71), PQPQQPFPQ (SEQ ID NO: 72), PIPQQPQPY (SEQ ID NO: 73), PFPQPQQPF (SEQ ID NO: 74), PQPQQPFPQ (SEQ ID NO: 75), PYPEQQEPF (SEQ ID NO: 76), PYPEQQQPF (SEQ ID NO: 77), PFSQQQQPV (SEQ ID NO: 78), QGSFQPSQQ (SEQ ID NO: 79), QQPQQPFPQ (SEQ ID NO: 80), QQPQQPYPQ (SEQ ID NO: 81), QQGYYPTSPQ (SEQ ID NO: 82), QGSFQPSQQ (SEQ ID NO: 83), PQQSFPQQQ (SEQ ID NO: 84), QGYYPTSPQ (SEQ ID NO: 85), LQPFPQPELPYPQPQ (SEQ ID NO: 86), QPFPQPQQPFPWQP (SEQ ID NO: 87), PQQPIPQQPQPYPQQ (SEQ ID NO: 88), or EQPIPQQPQ (SEQ ID NO: 107)).

A gluten peptide may also be an analog of any of the peptides described herein. Preferably, in some embodiments the analog is recognized by a CD4⁺ T cell that recognizes one or more of the epitopes listed herein. Exemplary analogs comprise a peptide that has a sequence that is, e.g., 75%, 80%, 85%, 90%, 95%, 96%, 97%, 98%, or 99% homologous to the epitopes specifically recited herein. In some embodiments, the analogs comprise a peptide that is, e.g., 75%, 80%, 85%, 90%, 95%, 96%, 97%, 98%, or 99% homologous to the peptides specifically recited herein. Analogs may also be a variant of any of the peptides provided, such variants can include conservative amino acid substitution variants, e.g., E to D substitution.

In some embodiments, analogs may include one or more amino acid substitutions as shown in Table A (see, e.g., Anderson et al. Antagonists and non-toxic variants of the dominant wheat gliadin T cell epitope in coeliac disease. Gut. 2006 April; 55(4): 485-491; and PCT Publication WO2003104273, the contents of which are incorporated herein by reference). The gluten peptides provided herein include analogs of SEQ ID NO:93 comprising one or more of the listed amino acid substitutions. In some embodiments, the analog is an analog of SEQ ID NO: 93 comprising one of the amino acid substitutions provided in Table A below.

TABLE A
Exemplary substitutions in the epitope FPQPELPYP (SEQ ID NO: 93)
Amino acid

| in epitope | F | P | Q | P | E | L | P | Y | P |
|---|---|---|---|---|---|---|---|---|---|
| Exemplary | A, G, H, I, | A, F, I, M, | A, F, G, | — | D | M | S | I, S | S, T, |
| Substitutions | L, M, P, S | S, T, V, | H, I, L, | | | | | V, W | Y |
| | T, W, Y | W, Y | M, S, T | | | | | | |
| | | | V | | | | | | |

The length of the peptide may vary. In some embodiments, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments, peptides are, e.g., 4-1000, 4-500, 4-100, 4-50, 4-40, 4-30, or 4-20 amino acids in length. In some embodiments, peptides are 4-20, 5-20, 6-20, 7-20, 8-20, 9-20, 10-20, 11-20, 12-20, 13-20, 14-20, or 15-20 amino acids in length. In some embodiments, peptides are e.g., 5-30, 10-30, 15-30 or 20-30 amino acids in length. In some embodiments, peptides are 4-50, 5-50, 6-50, 7-50, 8-50, 9-50, 10-50, 11-50, 12-50, 13-50, 14-50, or 15-50 amino acids in length. In some embodiments, peptides are 8-30 amino acids in length.

In some embodiments of any one of the methods or kits provided herein, a composition comprising at least one or one or more gluten peptide(s) is contemplated. In some embodiments of any one of the methods provided herein, the methods described herein comprise administering the composition to a subject (e.g., a subject having or suspected of having Celiac disease). In some embodiments of any one of the methods provided herein, the composition is formulated for intradermal administration to a subject. In some embodiments of any one of the methods provided herein, the composition is formulated as a bolus for intradermal injection to a subject. In some embodiments of any one of the methods or kits provided herein, the composition is formulated as a sterile, injectable solution. In some embodiments of any one of the methods or kits provided herein, the composition is formulated as a bolus for oral administration to a subject. In some embodiments, the sterile, injectable solution is sodium chloride. In some embodiments, the sodium chloride is sterile sodium chloride 0.9% USP.

In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), or (iii) a third peptide comprising the amino acid sequence PIPEQPQPY (SEQ ID NO: 5) and EQPIPEQPQ (SEQ ID NO: 106). “First”, “second”, and “third” are not meant to imply an order of use or importance, unless specifically stated otherwise. In some embodiments, the peptides are 8-30 amino acids in length. In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises LQPFPQPELPYPQPQ (SEQ ID NO: 6); the second peptide comprises QPFPQPEQPFPWQP (SEQ ID NO: 7); and/or the third peptide comprises PEQPIPEQPQPYPQQ (SEQ ID NO: 8).

In some embodiments, it may be desirable to utilize the non-deamidated forms of such peptides, e.g., if the peptides are contained within a composition for administration to a subject where tissue transglutaminase will act in situ (see, e.g., Øyvind Molberg, Stephen McAdam, Knut E. A. Lundin, Christel Kristiansen, Helene Arentz-Hansen, Kjell Kett and Ludvig M. Sollid. T cells from celiac disease lesions recognize gliadin epitopes deamidated in situ by endogenous tissue transglutaminase. Eur. J. Immunol. 2001. 31: 1317-1323). Accordingly, in some embodiments, the composition comprises at least one of: (i) a first peptide comprising the amino acid sequence PFPQPQLPY (SEQ ID NO: 19) and PQPQLPYPQ (SEQ ID NO: 20), (ii) a second peptide comprising the amino acid sequence PFPQPQQPF (SEQ ID NO: 21) and PQPQQPFPW (SEQ ID NO: 22), and (iii) a third peptide comprising the amino acid sequence PIPQQPQPY (SEQ ID NO: 23). In some embodiments, the composition comprises at least one of: (i) a first peptide comprising the amino acid sequence PFPQPQLPY (SEQ ID NO: 19) and PQPQLPYPQ (SEQ ID NO: 20), (ii) a second peptide comprising the amino acid sequence PFPQPQQPF (SEQ ID NO: 21) and PQPQQPFPW (SEQ ID NO: 22), or (iii) a third peptide comprising the amino acid sequence PIPQQPQPY (SEQ ID NO: 23) and EQPIPQQPQ (SEQ ID NO: 107). In some embodiments, the first peptide comprises LQPFPQPQLPYPQPQ (SEQ ID NO: 86); the second peptide comprises QPFPQPQQPFPWQP (SEQ ID NO: 87); and/or the third peptide comprises PQQPIPQQPQPYPQQ (SEQ ID NO: 88). In some embodiments, the peptides are 8-30 amino acids in length.

Modifications to a gluten peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, pyrolation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc.

The phrases “protecting group” and “blocking group” as used herein, refers to modifications to the peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- and/or C-terminal. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell.

A gluten peptide may also be chemically modified at the level of amino acid side chains, of amino acid chirality, and/or of the peptide backbone.

Particular changes can be made to a gluten peptide to improve resistance to degradation or optimize solubility properties or otherwise improve bioavailability compared to the parent gluten peptide, thereby providing gluten peptides having similar or improved therapeutic, diagnostic and/or pharmacokinetic properties. A preferred such modification includes the use of an N-terminal acetyl group or pyroglutamate and/or a C-terminal amide. Such modifications have been shown in the art to significantly increase the half-life and bioavailability of the peptides compared to the parent peptides having a free N- and C-terminus (see, e.g., PCT Publication No.: WO/2010/060155). In some embodiments, a gluten peptide comprises an N-terminal acetyl group or pyroglutamate group, and/or a C-terminal amide group. In some embodiments, the first, second and/or third peptides described above comprise an N-terminal acetyl group or pyroglutamate group, and/or a C-terminal amide group. In some embodiments, the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and/or the third peptide comprises EPEQPIPEQPQPYPQQ (SEQ ID NO: 11), wherein the N-terminal E is a pyroglutamate. In some embodiments, the first peptide comprises the amino acid sequence ELQPFPQPELPYPQPQ (SEQ ID NO: 9), wherein the N-terminal glutamate is a pyroglutamate and the C-terminal glutamine is amidated (e.g., the free C-terminal COO is amidated); the second peptide comprises the amino acid sequence EQPFPQPEQPFPWQP (SEQ ID NO: 10), wherein the N-terminal glutamate is a pyroglutamate and the C-terminal proline is amidated (e.g., the free C-terminal COO is amidated); and/or the third peptide comprises the amino acid sequence EPEQPIPEQPQPYPQQ (SEQ ID NO: 11), wherein the N-terminal glutamate is a pyroglutamate and the C-terminal glutamine is amidated (e.g., the free C-terminal COO is amidated). In some embodiments, the first peptide consists of the amino acid sequence ELQPFPQPELPYPQPQ (SEQ ID NO: 9), wherein the N-terminal glutamate is a pyroglutamate and the C-terminal glutamine is amidated (e.g., the free C-terminal COO is amidated); the second peptide consists of the amino acid sequence EQPFPQPEQPFPWQP (SEQ ID NO: 10), wherein the N-terminal glutamate is a pyroglutamate and the C-terminal proline is amidated (e.g., the free C-terminal COO is amidated); and/or the third peptide consists of the amino acid sequence EPEQPIPEQPQPYPQQ (SEQ ID NO: 11), wherein the N-terminal glutamate is a pyroglutamate and the C-terminal glutamine is amidated (e.g., the free C-terminal COO is amidated). In some embodiments, the composition comprises 30 micrograms of the peptides (i.e., 10 micrograms of the first peptide and an equimolar amount of each of the second and third peptides). In some embodiments, the composition comprises 45 micrograms of the peptides (i.e., 15 micrograms of the first peptide and an equimolar amount of each of the second and third peptides). In some embodiments, the composition comprises 60 micrograms of the peptides (i.e., 20 micrograms of the first peptide and an equimolar amount of each of the second and third peptides). In some embodiments, the composition comprises 150 micrograms of the peptides (i.e., 50 micrograms of the first peptide and an equimolar amount of each of the second and third peptides). In some embodiments, the composition comprises 300 micrograms of the peptides (i.e., 100 micrograms of the first peptide and an equimolar amount of each of the second and third peptides). Methods for producing equimolar peptide compositions are known in the art and provided herein (see, e.g., Muller et al. Successful immunotherapy with T-cell epitope peptides of bee venom phospholipase A2 induces specific T-cell anergy in patient allergic to bee venom. J. Allergy Clin. Immunol. Vol. 101, Number 6, Part 1: 747-754 (1998)).

### Other Testing

In some embodiments of any one of the methods provided, methods described herein further comprise other testing of a subject (e.g., based on the results of the methods described herein). As used herein, “other testing” describes use of at least one additional method, such as a diagnostic method, in addition to the methods provided herein. Any method, such as a diagnostic method, or combinations thereof for Celiac disease is contemplated as other testing. Exemplary other testing includes, but is not limited to, intestinal biopsy, serology (measuring the levels of one or more antibodies present in the serum), genotyping (see, e.g., Walker-Smith J A, et al. Arch Dis Child 1990), and measurement of a T cell response. Such other testing may be performed as part of the methods described herein or after the methods described herein (e.g., as a companion assay, such as a diagnostic), or before use of the methods described herein (e.g., as a first-pass screen to eliminate certain subjects before use of the methods described herein, e.g., eliminating those that do not have one or more HLA-DQA and HLA-DQB susceptibility alleles).

Detection of serum antibodies (serology) is also contemplated. The presence of such serum antibodies can be detected using methods known to those of skill in the art, e.g., by ELISA, histology, cytology, immunofluorescence or western blotting. Such antibodies include, but are not limited to: IgA ant-endomysial antibody (IgA EMA), IgA anti-tissue transglutaminase antibody (IgA tTG), IgA anti-deamidated gliadin peptide antibody (IgA DGP), and IgG anti-deamidated gliadin peptide antibody (IgG DGP).

IgA EMA: IgA endomysial antibodies bind to endomysium, the connective tissue around smooth muscle, producing a characteristic staining pattern that is visualized by indirect immunofluorescence. The target antigen has been identified as tissue transglutaminase (tTG or transglutaminase 2). IgA endomysial antibody testing is thought to be moderately sensitive and highly specific for untreated (active) Celiac disease.

IgA tTG: The antigen is tTG. Anti-tTG antibodies are thought to be highly sensitive and specific for the diagnosis of Celiac disease. Enzyme-linked immunosorbent assay (ELISA) tests for IgA anti-tTG antibodies are now widely available and are easier to perform, less observer-dependent, and less costly than the immunofluorescence assay used to detect IgA endomysial antibodies. The diagnostic accuracy of IgA anti-tTG immunoassays has been improved further by the use of human tTG in place of the nonhuman tTG preparations used in earlier immunoassay kits. Kits for IgA tTG are commercially available (INV 708760, 704525, and 704520, INOVA Diagnostics, San Diego, Calif.).

Deamidated gliadin peptide-IgA (DGP-IgA) and deamidated gliadin peptide-IgG (DGP-IgG) are also contemplated herein and can be evaluated with commercial kits (INV 708760, 704525, and 704520, INOVA Diagnostics, San Diego, Calif.).

Genetic testing (genotyping) is also contemplated. Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRB5 & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256.; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

T cell response tests are also contemplated as other testing. In some embodiments, a T cell response test comprises contacting a sample comprising a T cell with a gluten peptide and measuring a T cell response in the sample. In some embodiments, a T cell response is measured by measuring a level of IFN-γ, where an increased level of IFN-γ compared to a control level (e.g., a level of IFN-γ in a sample that has not been contacted with a gluten peptide) may identify a subject as having Celiac disease. T cell response tests are known in the art (see, e.g., PCT Publication Nos.: WO/2001/025793, WO/2003/104273, WO/2005/105129, and WO/2010/060155).

### Treatment

In some embodiments of any one of the methods provided herein, the methods described herein further comprise a treatment step, such as treating a subject identified as having or likely as having Celiac disease. In some embodiments of any one of the methods provided, the methods comprise a step where information regarding treatment is provided to the subject. Such information can be given orally or in written form, such as with written materials. Written materials may be in an electronic form. Any known treatment of Celiac disease is contemplated herein. Exemplary treatments include, e.g., a gluten-free diet. Other exemplary treatments include endopeptidases, such as ALV003 (Alvine) and AT1001 (Alba), agents that inhibit transglutaminase activity, agents that block peptide presentation by HLA DQ2.5, or oral resins that bind to gluten peptides and reduce their bioavailability.

Compositions comprising gluten peptides for use in treating Celiac disease are known in the art (see, e.g., PCT Publication Nos.: WO/2001/025793, WO/2003/104273, WO/2005/105129, and WO/2010/060155, which are incorporated herein by reference in their entirety). In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5). In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) and EQPIPEQPQ (SEQ ID NO: 106). In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises the amino acid sequence LQPFPQPELPYPQPQ (SEQ ID NO: 6); the second peptide comprises the amino acid sequence QPFPQPEQPFPWQP (SEQ ID NO: 7); and/or the third peptide comprises the amino acid sequence PEQPIPEQPQPYPQQ (SEQ ID NO: 8). Modifications to such peptides, e.g., an N-terminal pyro-glutamate and/or C-terminal amide, are contemplated and described herein. In some embodiments, the first peptide comprises the amino acid sequence ELQPFPQPELPYPQPQ (SEQ ID NO: 9), wherein the N-terminal glutamate is a pyroglutamate and the C-terminal glutamine is amidated (e.g., the free C-terminal COO is amidated); the second peptide comprises the amino acid sequence EQPFPQPEQPFPWQP (SEQ ID NO: 10), wherein the N-terminal glutamate is a pyroglutamate and the C-terminal proline is amidated (e.g., the free C-terminal COO is amidated); and/or the third peptide comprises the amino acid sequence EPEQPIPEQPQPYPQQ (SEQ ID NO: 11), wherein the N-terminal glutamate is a pyroglutamate and the C-terminal glutamine is amidated (e.g., the free C-terminal COO is amidated). In some embodiments, the first peptide consists of the amino acid sequence ELQPFPQPELPYPQPQ (SEQ ID NO: 9), wherein the N-terminal glutamate is a pyroglutamate and the C-terminal glutamine is amidated (e.g., the free C-terminal COO is amidated); the second peptide consists of the amino acid sequence EQPFPQPEQPFPWQP (SEQ ID NO: 10), wherein the N-terminal glutamate is a pyroglutamate and the C-terminal proline is amidated (e.g., the free C-terminal COO is amidated); and/or the third peptide consists of the amino acid sequence EPEQPIPEQPQPYPQQ (SEQ ID NO: 11), wherein the N-terminal glutamate is a pyroglutamate and the C-terminal glutamine is amidated (e.g., the free C-terminal COO is amidated). In some embodiments, the composition comprises 150 micrograms of the peptides (i.e., 50 micrograms of the first peptide and an equimolar amount of each of the second and third peptides). In some embodiments, the composition comprises 300 micrograms of the peptides (i.e., 100 micrograms of the first peptide and an equimolar amount of each of the second and third peptides).

Treatments may be administered through any method known in the art. Pharmaceutical compositions suitable for each administration route are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005). In some embodiments, a treatment, e.g., a composition comprising a gluten peptide, is administered via injection, such as intradermal injection.

The peptides or other compositions provided herein may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. Compositions, such as pharmaceutical compositions, may include a pharmaceutically acceptable carrier. The term “pharmaceutically acceptable carrier” refers to molecular entities and compositions that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbents, dispersion media, coatings, stabilizers, protective colloids, adhesives, thickeners, thixotropic agents, penetration agents, sequestering agents, isotonic and absorption delaying agents that do not affect the activity of the active agents of the pharmaceutical composition. The carrier can be any of those conventionally used and is limited only by chemico-physical considerations, such as solubility and lack of reactivity with the active agent, and by the route of administration. Suitable carriers for the pharmaceutical composition include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers. Other carriers are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005).

The pharmaceutical composition(s) may be in the form of a sterile injectable aqueous or oleagenous suspension. In some embodiments, the composition is formulated as a sterile, injectable solution. This suspension or solution may be formulated according to known methods using those suitable dispersing or wetting agents and suspending agents which have been mentioned above. The sterile injectable preparation may be a suspension in a non-toxic parenterally-acceptable diluent or solvent, for example as a solution in 1,3-butanediol. Among the acceptable carriers that may be employed are water, Ringer's solution and isotonic sodium chloride solution. In some embodiments, the composition is formulated as a sterile, injectable solution, wherein the solution is a sodium chloride solution (e.g., sodium chloride 0.9% USP). In some embodiments, the composition is formulated as a bolus for intradermal injection. Examples of appropriate delivery mechanisms for intradermal administration include, but are not limited to, syringes, needles, and osmotic pumps.

It can be advantageous to formulate the active agent in a dosage unit form for ease of administration and uniformity of dosage. “Dosage unit form” as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of subjects. Alternatively, the compositions may be presented in multi-dose form. Examples of dosage units include sealed ampoules and vials and may be stored in a freeze-dried condition requiring only the addition of the sterile liquid carrier immediately prior to use.

The actual amount administered (or dose or dosage) and the rate and time-course of administration will depend on the nature and severity of the condition being treated as well as the characteristics of the subject to be treated (weight, age, etc.). Prescription of treatment, for example, decisions on dosage, timing, frequency, etc., is within the responsibility of general practitioners or specialists (including human medical practitioner, veterinarian or medical scientist) and typically takes account of the disorder to be treated, the condition of the subject, the site of delivery, the method of administration and other factors known to practitioners. Examples of techniques and protocols can be found in, e.g., Remington's Pharmaceutical Sciences, 16th Ed. Mack Publishing Company, 1980 and Remington: The Science and Practice of Pharmacy, 21st Ed. Lippincott Williams & Wilkins, 2005. Effective amounts may be measured from ng/kg body weight to g/kg body weight per minute, hour, day, week or month.

As used herein, the terms “treat”, “treating”, and “treatment” include abrogating, inhibiting, slowing, or reversing the progression of a disease or condition, or ameliorating or preventing a clinical symptom of the disease (for example, Celiac disease). Treatment may include induction of immune tolerance (for example, to gluten or peptides thereof), modification of the cytokine secretion profile of the subject and/or induction of suppressor T cell subpopulations to secrete cytokines. Thus, a subject treated according to the disclosure preferably, in some embodiments, is able to eat at least wheat, rye, and/or barley without a significant T cell response which would normally lead to symptoms of Celiac disease. In some embodiments, an effective amount of a treatment is administered. The term “effective amount” means the amount of a treatment sufficient to provide the desired therapeutic or physiological effect when administered under appropriate or sufficient conditions.

Toxicity and therapeutic efficacy of the agent can be determined by standard pharmaceutical procedures in cell cultures or experimental animals by determining the IC50 and the maximal tolerated dose. The data obtained from these cell culture assays and animal studies can be used to formulate a range suitable for humans.

### Kits

Another aspect of the disclosure relates to kits. In some embodiments, the kit comprises a composition comprising a gluten peptide as described herein. In some embodiments, the kit comprises: (a) a composition comprising at least one gluten peptide, and (b) a binding partner for a circulating cytokine or chemokine described herein. In some embodiments, the kit comprises: (a) a composition comprising at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), or (iii) a third peptide comprising the amino acid sequence PIPEQPQPY (SEQ ID NO: 5) or comprising the amino acid sequence PIPEQPQPY (SEQ ID NO: 5) and EQPIPEQPQ (SEQ ID NO: 106); and (b) a binding partner for a circulating cytokine or chemokine described herein. In some embodiments, the kit comprises: (a) a composition comprising (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or comprising the amino acid sequence PIPEQPQPY (SEQ ID NO: 5) and EQPIPEQPQ (SEQ ID NO: 106); and (b) a binding partner for a circulating cytokine or chemokine described herein. In some embodiments, the kit comprises more than one binding partner for more than one circulating cytokine or chemokine, e.g., 2, 3, 4, 5, 6, 7, 8, 9, 10, or more binding partners for 2, 3, 4, 5, 6, 7, 8, 9, 10, or more circulating cytokines or chemokines. In some embodiments, the binding partner is an antibody. In some embodiments of any one of the kits provided, the kit further comprises an agent that recognizes the binding partner for the cytokine or chemokine. In some embodiments, the agent is an antibody. In some embodiments, the composition contained in the kit is contained within a container suitable for injection of the composition into a subject, e.g., depot, syringe, or osmotic pump. In some embodiments of any one of the kits provided, the kit further comprises a container suitable for injection of the composition into a subject, e.g., depot, syringe, or osmotic pump.

In some embodiments, the kit comprises a composition comprising a gluten peptide as described herein and a binding partner for a circulating cytokine or chemokine and/or circulating T cell as provided herein. In some embodiments, the kit comprises: (a) a composition comprising at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or comprising the amino acid sequence PIPEQPQPY (SEQ ID NO: 5) and EQPIPEQPQ (SEQ ID NO: 106); and (b) a binding partner for a circulating cytokine or chemokine and/or a circulating T cell as described herein. In some embodiments, the kit comprises: (a) a composition comprising (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), and (iii) a third peptide comprising the amino acid sequence PIPEQPQPY (SEQ ID NO: 5) or comprising the amino acid sequence PIPEQPQPY (SEQ ID NO: 5) and EQPIPEQPQ (SEQ ID NO: 106); and (b) a binding partner for a circulating cytokine or chemokine described herein. In some embodiments, the kit comprises more than one binding partner for more than one circulating cytokine or chemokine and/or circulating T cell. In some embodiments, the binding partner is an antibody or a MHC tetramer. In some embodiments, the kit further comprises an agent that recognizes the binding partner. In some embodiments, the agent is an antibody. In some embodiments, the composition contained in the kit is contained within a container suitable for injection of the composition into a subject, e.g., an implant, depot, syringe, or osmotic pump. In some embodiments, the composition contained in the kit is a composition suitable for oral administration, e.g., a foodstuff.

In some embodiments of any one of the kits herein, the composition contained in the kit comprises the first and second peptide, the first and third peptide, or the second and third peptide. In some embodiments, the composition comprises the first and second peptide. In some embodiments, the composition comprises the first, second, and third peptide. In some embodiments, the first peptide comprises LQPFPQPELPYPQPQ (SEQ ID NO: 6); the second peptide comprises QPFPQPEQPFPWQP (SEQ ID NO: 7); and/or the third peptide comprises PEQPIPEQPQPYPQQ (SEQ ID NO: 8). In some embodiments, the first, second and/or third peptides comprise an N-terminal acetyl group or pyroglutamate group, and/or a C terminal amide group. In some embodiments, the first peptide comprises the amino acid sequence ELQPFPQPELPYPQPQ (SEQ ID NO: 9), wherein the N-terminal glutamate is a pyroglutamate and the C-terminal glutamine is amidated (e.g., the free C-terminal COO is amidated); the second peptide comprises the amino acid sequence EQPFPQPEQPFPWQP (SEQ ID NO: 10), wherein the N-terminal glutamate is a pyroglutamate and the C-terminal proline is amidated (e.g., the free C-terminal COO is amidated); and/or the third peptide comprises the amino acid sequence EPEQPIPEQPQPYPQQ (SEQ ID NO: 11), wherein the N-terminal glutamate is a pyroglutamate and the C-terminal glutamine is amidated (e.g., the free C-terminal COO is amidated). In some embodiments, the composition comprises 30 micrograms of the peptides (i.e., 10 micrograms of the first peptide and an equimolar amount of each of the second and third peptides). In some embodiments, the composition comprises 45 micrograms of the peptides (i.e., 15 micrograms of the first peptide and an equimolar amount of each of the second and third peptides). In some embodiments, the composition comprises 60 micrograms of the peptides (i.e., 20 micrograms of the first peptide and an equimolar amount of each of the second and third peptides). In some embodiments, the composition comprises 150 micrograms of the peptides (i.e., 50 micrograms of the first peptide and an equimolar amount of each of the second and third peptides).

In some embodiments of any one of the kits herein, the composition comprising a gluten peptide is contained within a container in the kit. In some embodiments, the composition is contained within a solution separate from the container, such that the composition may be added to the container prior to administration. In some embodiments, the composition is in lyophilized form in a separate container, such that the composition may be reconstituted and added to the container prior to administration, in some embodiments. In some embodiments, the container is present in the kit in duplicate or triplicate.

In some embodiments of any one of the kits herein, the kit further comprises a container suitable for injection of a composition to a subject, e.g., a syringe. In some embodiments, the kit further comprises a container for containing a sample obtained from a subject, e.g., a plasma, serum or urine sample. Containers for plasma, urine serum, etc. are known in the art and are commercially available (e.g., a Vacutainer™ or urine collection container from Becton Dickinson). In some embodiments, the container further contains an anti-coagulant, such as heparin, EDTA, citrate, sodium polyanethol sulfonate, or oxalate. In some embodiments, the container is structured to hold a defined volume, e.g., 1 mL or 5 mL. In some embodiments, the container is present in the kit in duplicate or triplicate.

In some embodiments, the kit further comprises a negative control, e.g., a composition that does not comprise a gluten peptide, e.g., a saline solution. In some embodiments, the kit further comprises a positive control, e.g., a composition comprising a chemokine, cytokine, or T cell at a known concentration or level.

In some embodiments, the kit comprises any combination of the components mentioned above.

Any suitable binding partner is contemplated. In some embodiments, the binding partner is any molecule that binds specifically to a circulating cytokine or chemokine, or a circulating T cell as provided herein. Any one of the kits provided may include binding partners for any one of the embodiments of pairs or sets of circulating cytokines or chemokines as provided herein. As described herein, “binds specifically” means that the molecule is more likely to bind to a portion of or the entirety of an antigen to be measured than to a portion of or the entirety of another molecule. In some embodiments, the binding partner is an antibody. As used herein, the term “antibody” also includes antigen-binding fragments thereof, such as Fab, F(ab)2, Fv, single chain antibodies, Fab and sFab fragments, F(ab′)2, Fd fragments, scFv, or dAb fragments. Methods for producing antibodies are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically. Methods for producing peptide molecules and aptamers are well known in the art (see, e.g., published US Patent Application No. 2009/0075834, U.S. Pat. Nos. 7,435,542, 7,807,351, and 7,239,742). In some embodiments, the binding partner is an MHC tetramer.

In some embodiments, the binding partner is any molecule that binds specifically to an cytokine or chemokine nucleic acid, such as mRNA. As described herein, “binds specifically to an mRNA” means that the molecule is more likely to bind to a portion of or the entirety of the mRNA to be measured (e.g., by complementary base-pairing) than to a portion of or the entirety of another mRNA or other nucleic acid. In some embodiments, the binding partner that binds specifically to an mRNA is a nucleic acid, e.g., a probe.

In some embodiments of any one of the kits provided, the kit further comprises a first and second binding partner for a cytokine or chemokine provided herein. In some embodiments, the first and second binding partners are antibodies or antigen binding fragments thereof. In some embodiments, the first and second binding partners are nucleic acids, such as nucleic acid probes. In some embodiments, the first or second binding partner is bound to a surface. The first or second binding partner may be bound to the surface covalently or non-covalently. The first or second binding partner may be bound directly to the surface, or may be bound indirectly, e.g., through a linker. Examples of linkers, include, but are not limited to, carbon-containing chains, polyethylene glycol (PEG), nucleic acids, monosaccharide units, and peptides. The surface can be made of any material, e.g., metal, plastic, paper, or any other polymer, or any combination thereof. In some embodiments, the first binding partner is washed over the cytokine bound to the second binding partner (e.g., as in a sandwich ELISA). The first binding partner may comprise a detectable label, or an agent that recognizes the first binding partner (e.g., a secondary antibody) may comprise a detectable label.

Any suitable agent that recognizes a binding partner is contemplated. In some embodiments, the binding partner is any molecule that binds specifically to the binding partner. In some embodiments, the agent is an antibody (e.g., a secondary antibody). Agents also include other peptide molecules and aptamers that bind specifically to a binding partner. In some embodiments, the binding partner comprises a biotin moiety and the agent is a composition that binds to the biotin moiety (e.g., an avidin or streptavidin). In some embodiments, the agent is a nucleic acid, such as a complementary nucleic acid.

In some embodiments, the binding partner and/or the agent comprise a detectable label. Any suitable detectable label is contemplated. Detectable labels include any composition detectable by spectroscopic, photochemical, biochemical, immunochemical, chemical, or other physical means, e.g., an enzyme, a radioactive label, a fluorophore, an electron dense reagent, biotin, digoxigenin, or a hapten. Such detectable labels are well-known in the art are detectable through use of, e.g., an enzyme assay, a chromogenic assay, a luminometric assay, a fluorogenic assay, or a radioimmune assay. The reaction conditions to perform detection of the detectable label depend upon the detection method selected.

In some embodiments of any one of the methods provided, the kit further comprises instructions for performing any one of the methods provided herein and/or for detecting a level of at least one circulating cytokine or chemokine and/or circulating T cell in a sample from a subject having or suspected of having Celiac disease or a subject undergoing treatment for Celiac disease. In some embodiments of any one of the kits provided, the instructions include the methods described herein. Instructions can be in any suitable form, e.g., as a printed insert or a label.

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, J. Perbal, A Practical Guide to Molecular Cloning, John Wiley and Sons (1984); J. Sambrook et al., Molecular Cloning: A Laboratory Manual, Cold Spring Harbour Laboratory Press (1989); T. A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); F. M. Ausubel et al. (editors), Current Protocols in Molecular Biology, Greene Pub. Associates and Wiley-Interscience (1988, including all updates until present); Ed Harlow and David Lane (editors) Antibodies: A Laboratory Manual, Cold Spring Harbour Laboratory, (1988); and J. E. Coligan et al. (editors), Current Protocols in Immunology, John Wiley & Sons (including all updates until present).

In any one aspect or embodiment provided herein “comprising” may be replaced with “consisting essentially of” or “consisting of”.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Methods

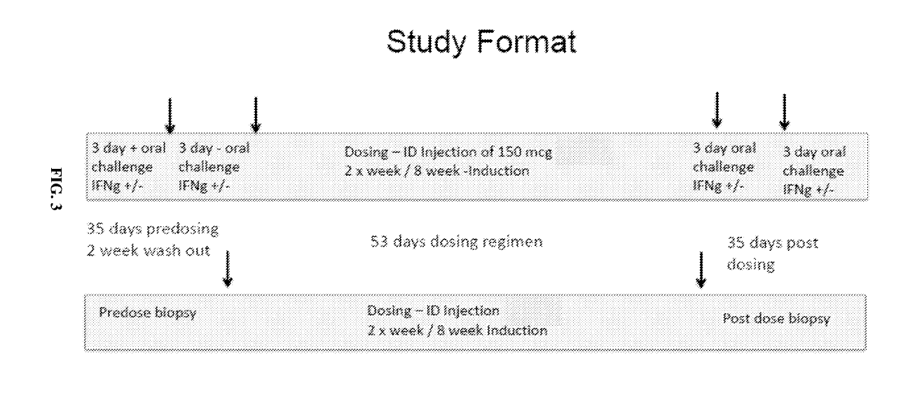

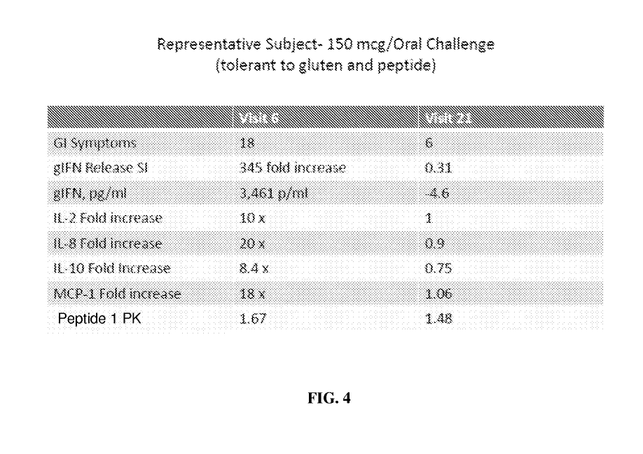

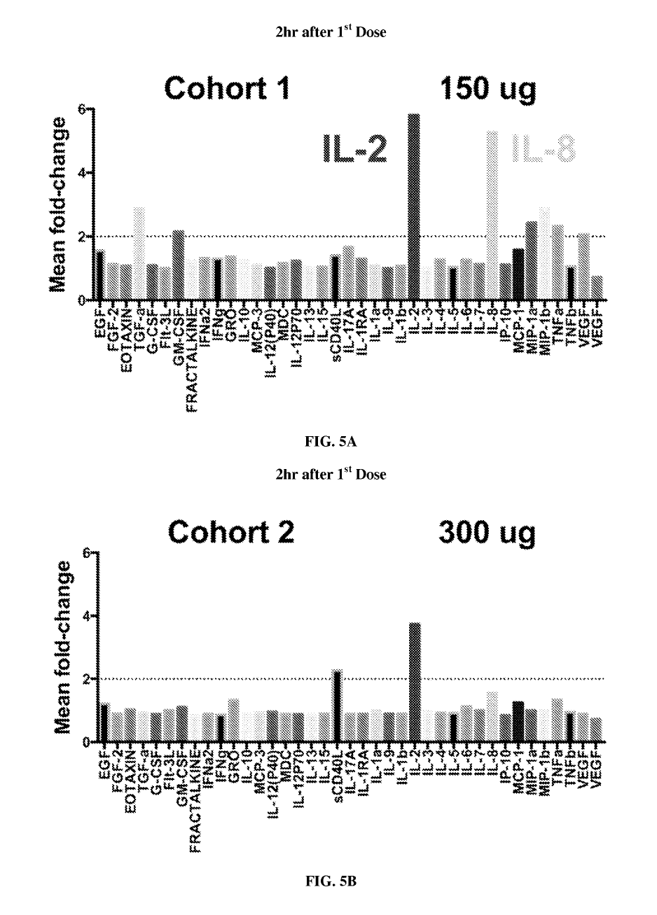

All subjects were HLA-DQ2.5+, followed strict gluten free diet, and had celiac disease. Subjects in Cohort 1 and Cohort 2 were dosed with a peptide composition 4 or 5 weeks following 3-day oral challenge with gluten 9 g/day. 0.1 mL of the peptide composition was administered intradermally to 8 subjects in Cohort 1 (150 μg), 10 subjects in Cohort 2 (300 μg), and 7 in Cohort 7 (150 μg). Placebo (0.1 mL normal saline) was administered intradermally to 4 subjects in Cohort 1, 3 in Cohort 2 and 7 in Cohort 7. The dosage regimen is shown in FIG. 3. The peptide composition administered included 3 peptides in sodium chloride 0.9% USP: ELQPFPQPELPYPQPQ (SEQ ID NO: 9), EQPFPQPEQPFPWQP (SEQ ID NO: 10), and EPEQPIPEQPQPYPQQ (SEQ ID NO: 11). For each peptide in the composition, the N-terminal glutamate was a pyroglutamate and the carboxyl group of the C-terminal proline or glutamine was amidated. The doses were either 150 micrograms (Cohort 1 and 7) or 300 micrograms (Cohort 2) of an equimolar mixture of the three peptides. Plasma samples were collected from the subjects in each cohort multiple times, pre- and post-first dose of the peptide composition (visit 6) and pre- and post-last dose of the peptide composition (visit 21). Cytokines and chemokines levels were measured in each plasma sample using the MAGPIX® multiplexing platform (Luminex, Tex., USA). The cytokines and chemokines measured were EGF, FGF-2, EOTAXIN, TGF-a, G-CSF, Flt-3L, GM-CSF, FRACTALKINE, IFNa2, IFNg, GRO, IL-10, MCP-3, IL-12(P40), MDC, IL-12P70, IL-13, IL-15, sCD40L, IL-17A, IL-1RA, IL-1a, IL-9, IL-1b, IL-2, IL-3, IL-4, IL-5, IL-6, IL-7, IL-8, IP-10, MCP-1, MIP-1a, MIP-1b, TNFa, TNFb, and VEGF.

An ex vivo whole blood cytokine release assay was performed pre- and post-treatment with the the peptide composition. Blood was collected 6 days after commencing 3-day oral challenge with gluten (approximately 9 g/day). The MAGPIX® assay discussed above was also used to confirm elevated IFN-γ plasma levels in blood incubated with the three constituent peptides (0.05 mg/mL/peptide), and also to show that levels of interleukin-2 and IFN-γ-inducible protein (IP-10) correlated with elevated concentrations of IFN-γ. Pretreatment gluten challenge was 4-5 weeks prior to commencing dosing. Post-treatment 3-day gluten challenge was commenced the day after the last dose.

### Results

Plasma cytokines and chemokines were measured from plasma samples collected at several time points pre- and post- the first and last dose of the peptide composition (visits 6 and 21).

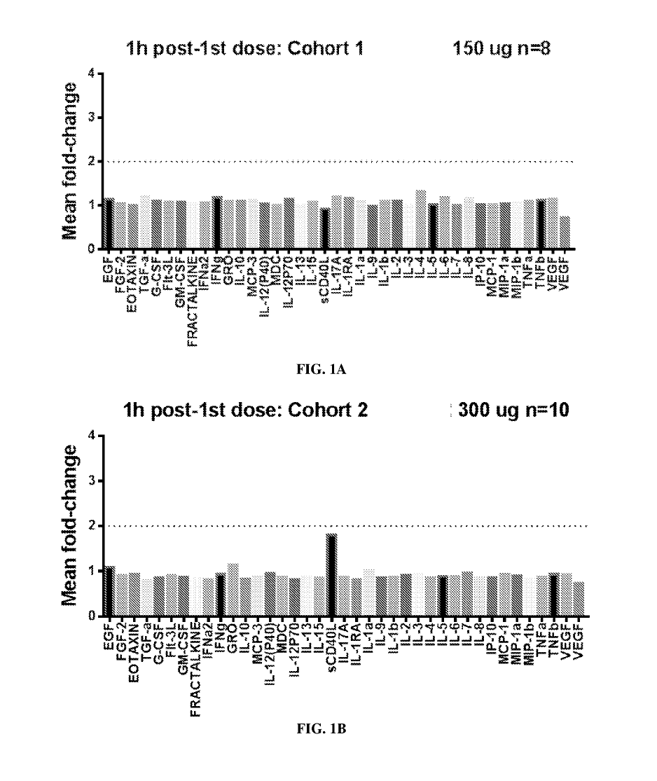

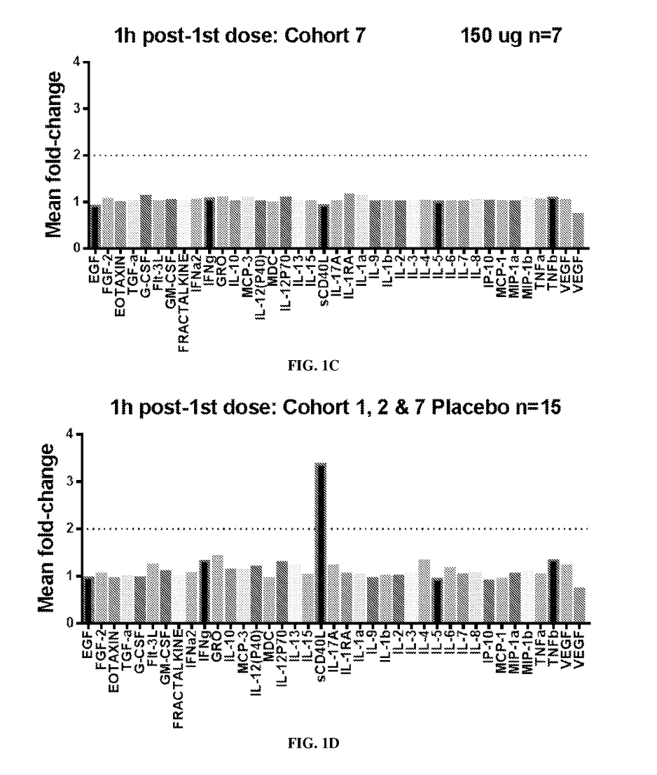

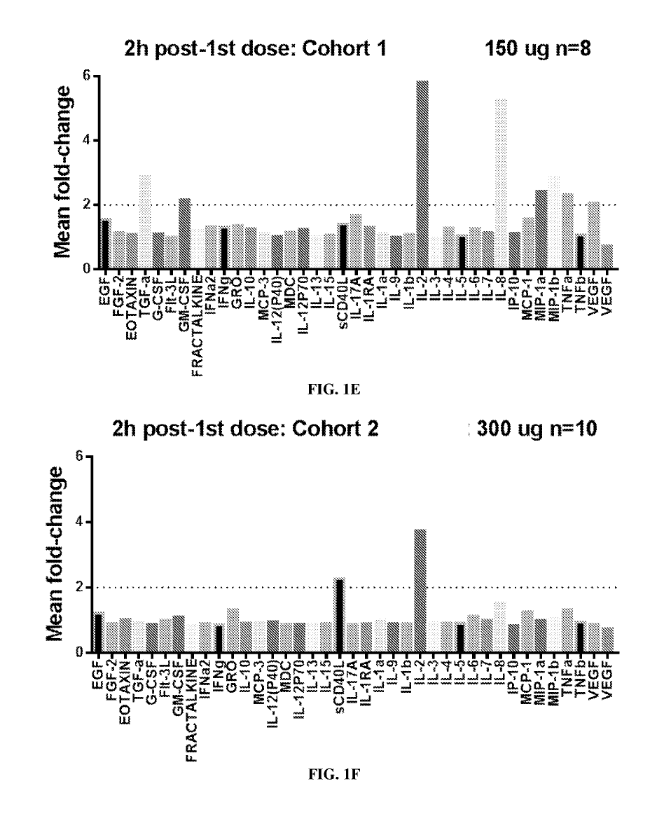

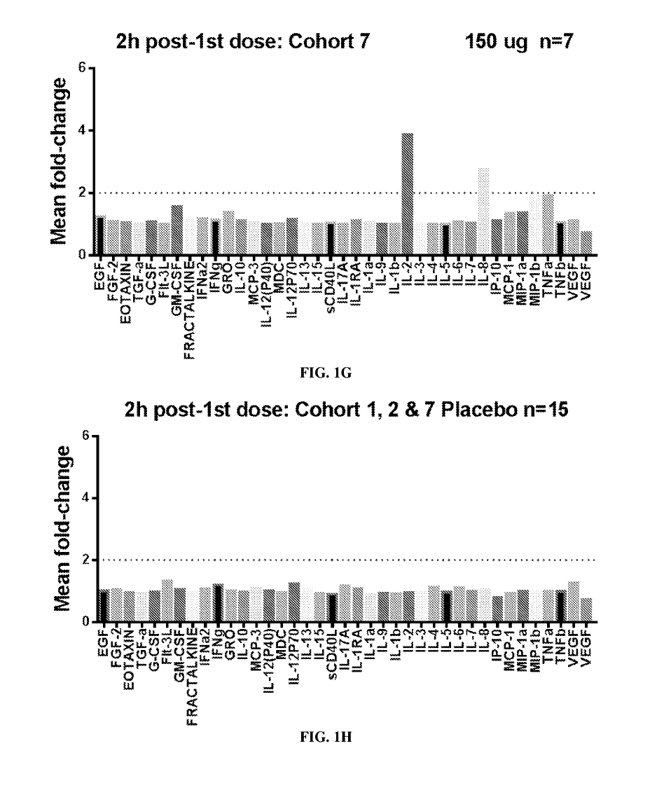

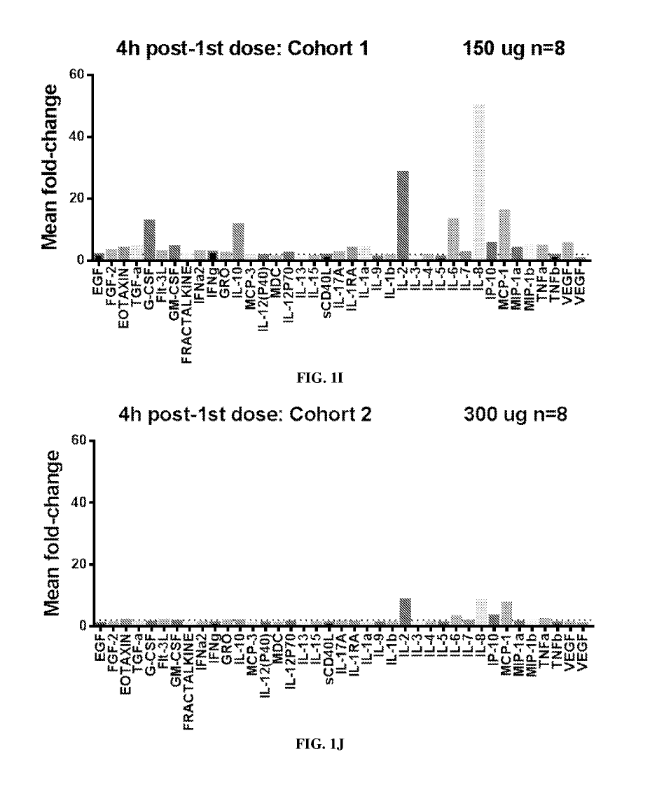

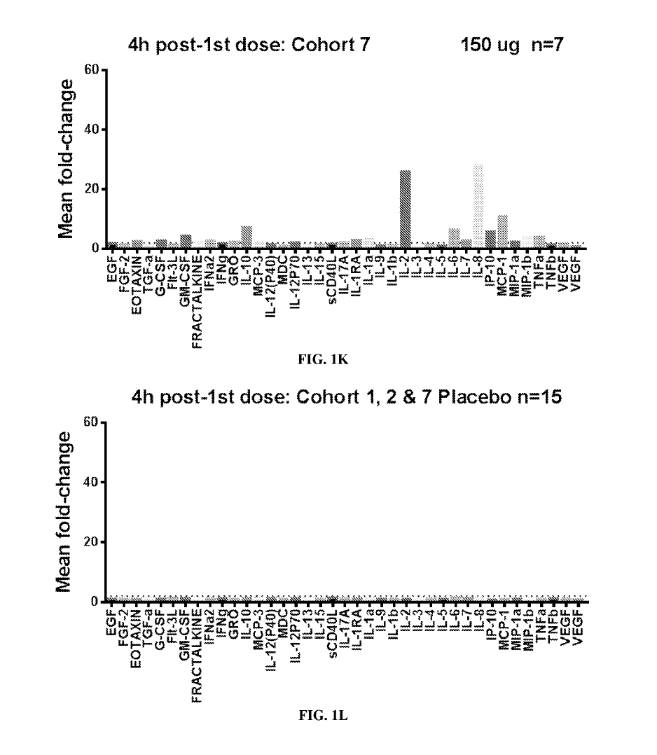

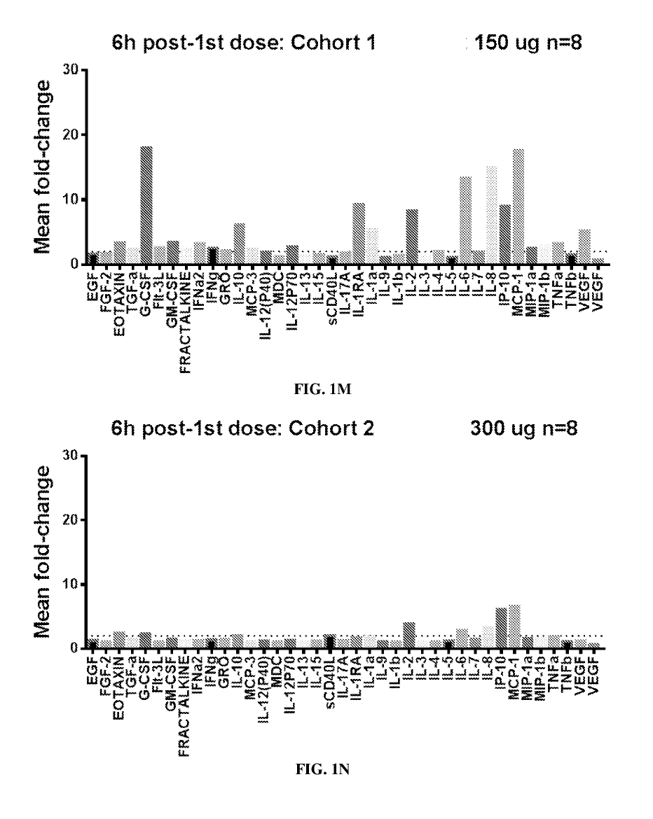

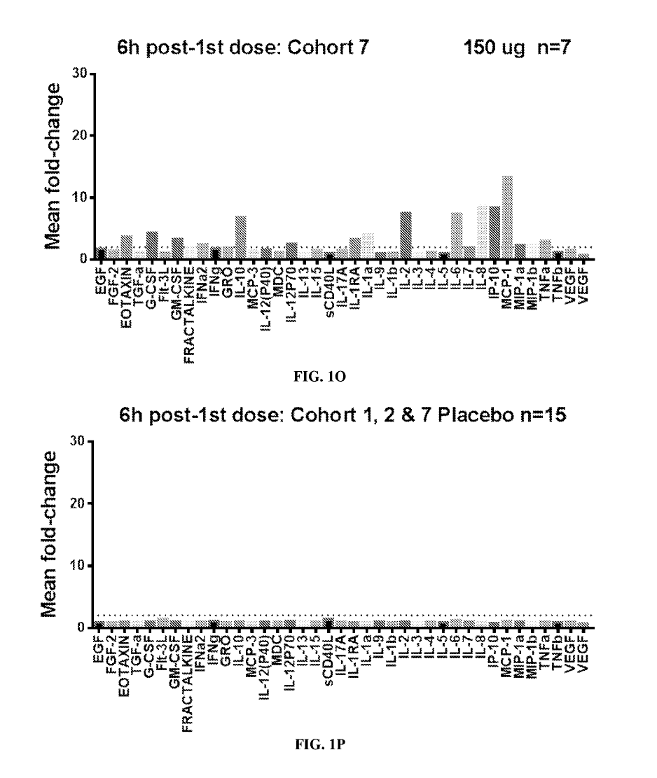

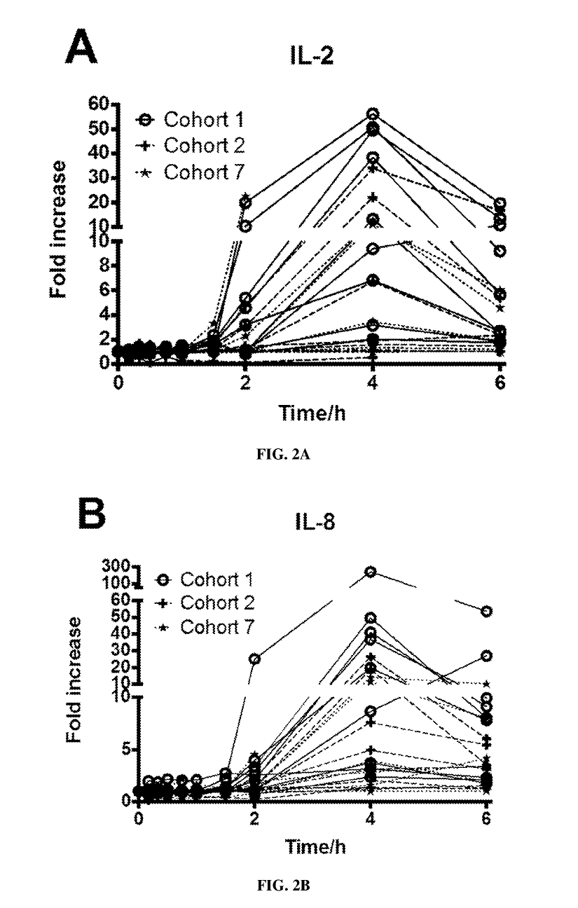

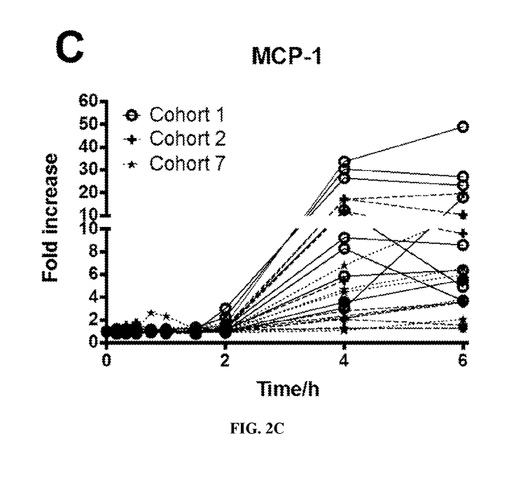

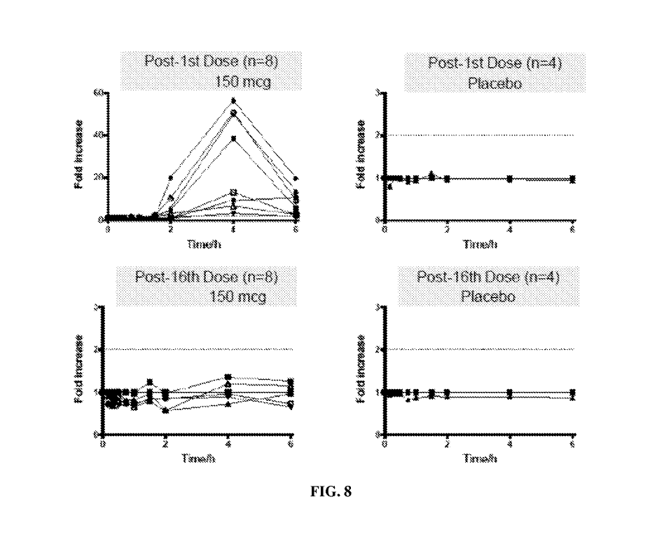

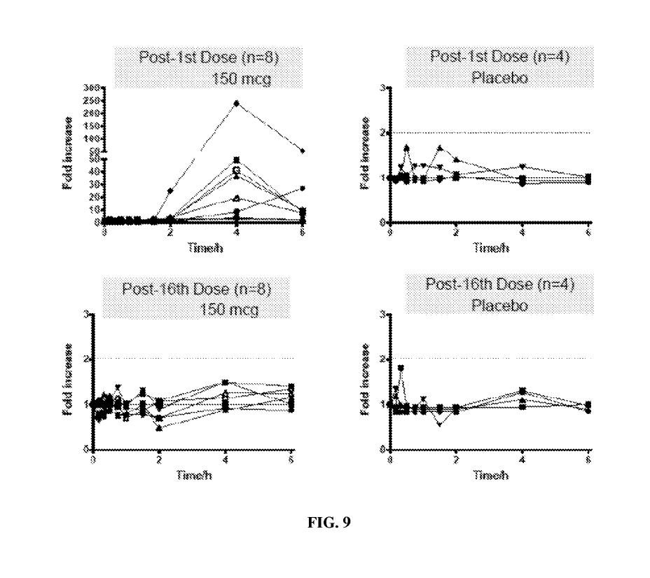

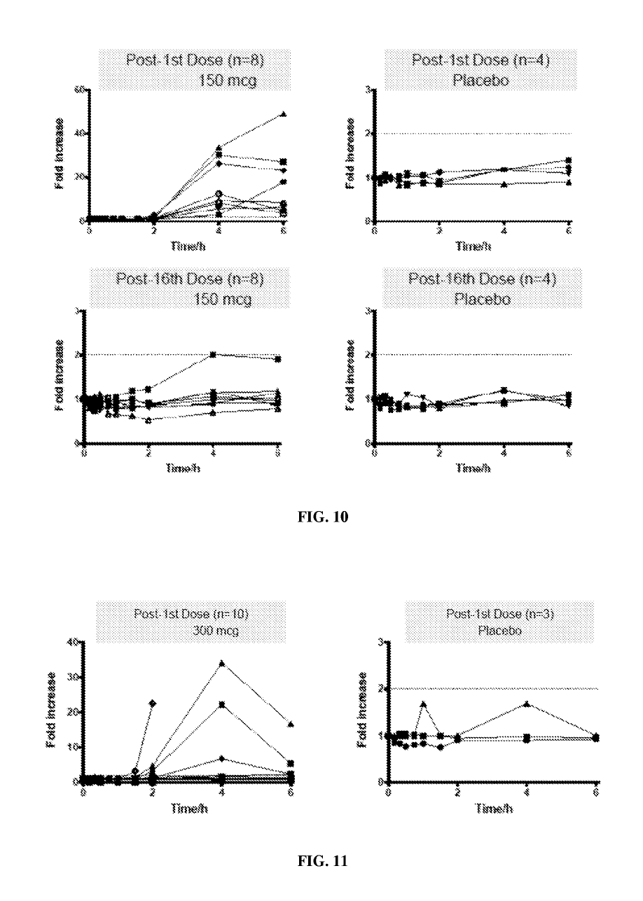

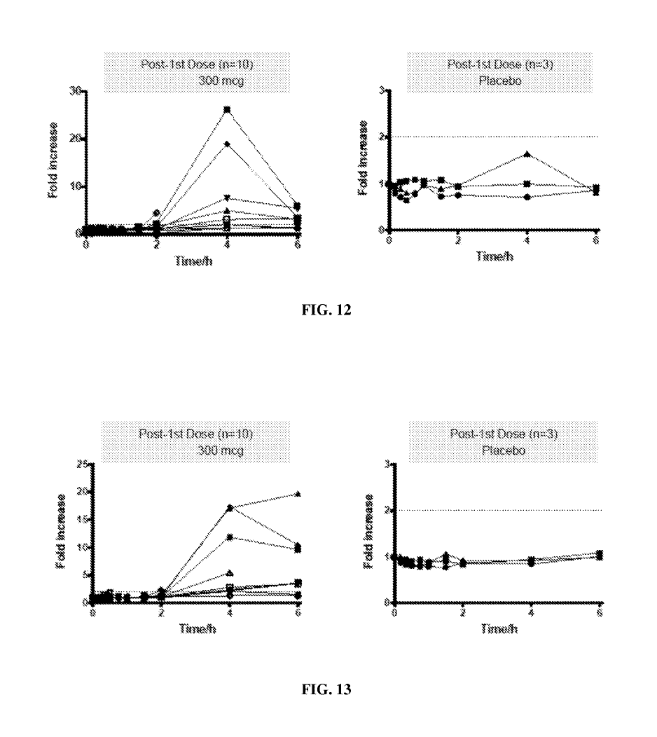

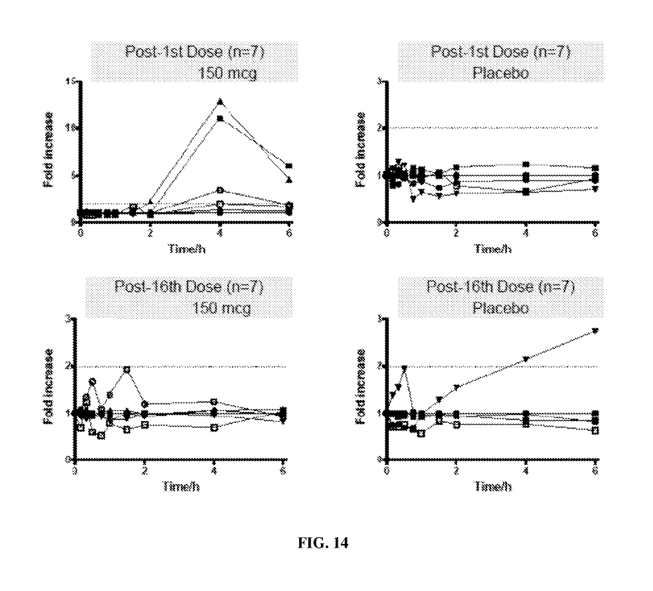

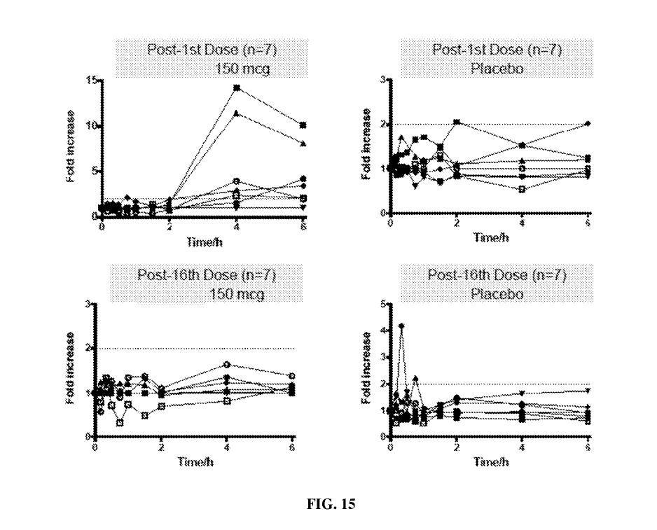

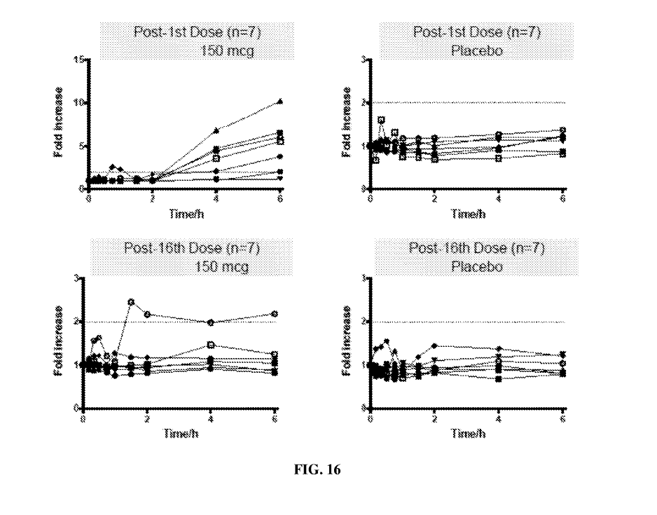

A rise in the level of several cytokines and chemokines was observed after the first dose of the peptide composition (visit 6) was administered but not after placebo was administered (FIG. 1, Tables 2-5). In particular, plasma levels of interleukin IL-2, IL-8, and MCP-1 showed pronounced elevations in HLA-DQ2.5+ celiac disease patients on gluten free diet following administration of the peptide composition (FIG. 2). Levels of IL-10 were also increased significantly. Further data showed that after the first dose (visit 6) was administered, IL-2 peaked early and IL-8, MCP-1, and IL-6 peaked subsequent to IL-2 (FIGS. 8-16). This elevation of these cytokines/chemokines did not occur in subjects receiving placebo (FIGS. 8-16). In a representative subject from Cohort 1 (150 micrograms of the peptide composition), the levels of IL-2, IL-8, IL-10 and MCP-1 were assessed. It was found that plasma IL-2 levels increased 10-fold, plasma I IL-8 levels increased 20-fold, plasma IL-10 levels increased 8.4-fold, and plasma MCP-1 levels increased by 18-fold after administration of the first dose of the peptide composition (FIG. 4). These results suggest that circulating cytokines and chemokines may be used to identify a subject having Celiac disease after administration of a gluten peptide-containing composition because subjects having Celiac disease had elevated levels of several cytokines and chemokines after administration of the first dose of the peptide composition but not after administration of a placebo or after repeated doses of the peptide composition.

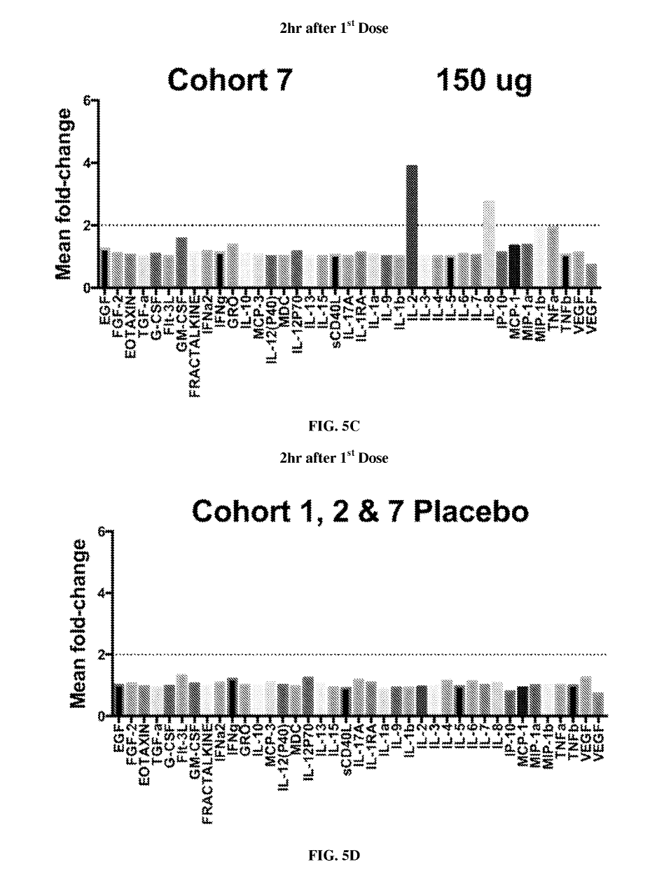

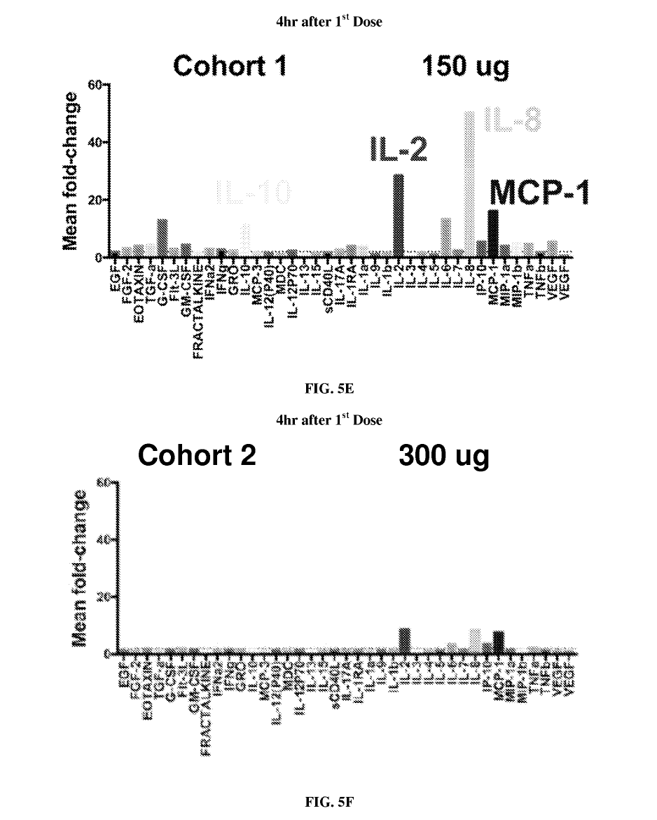

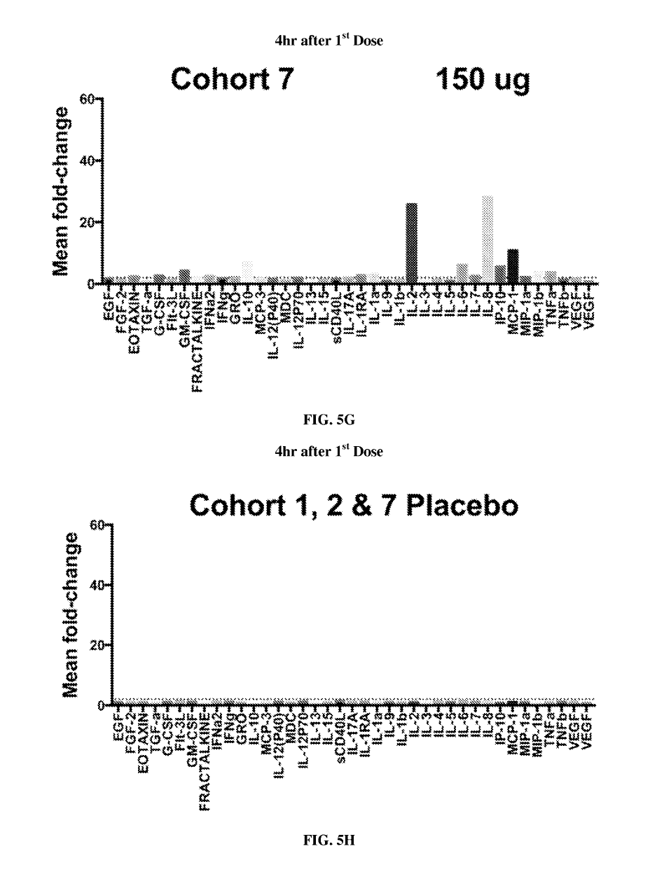

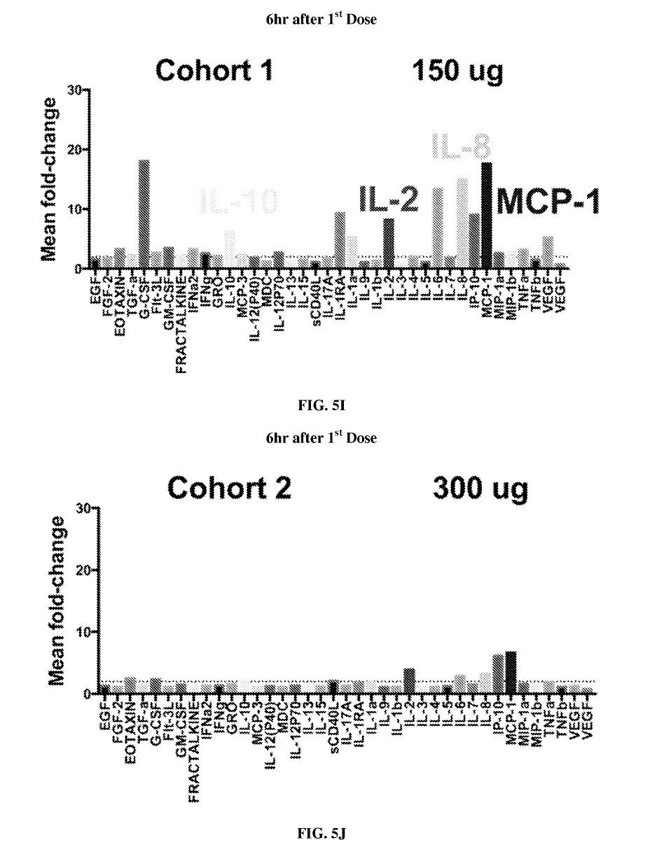

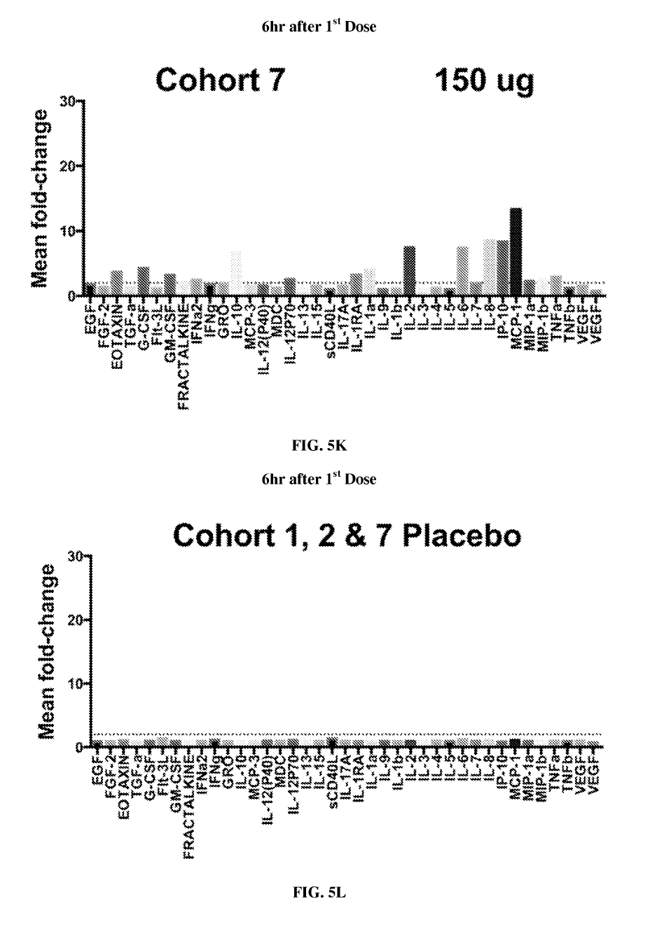

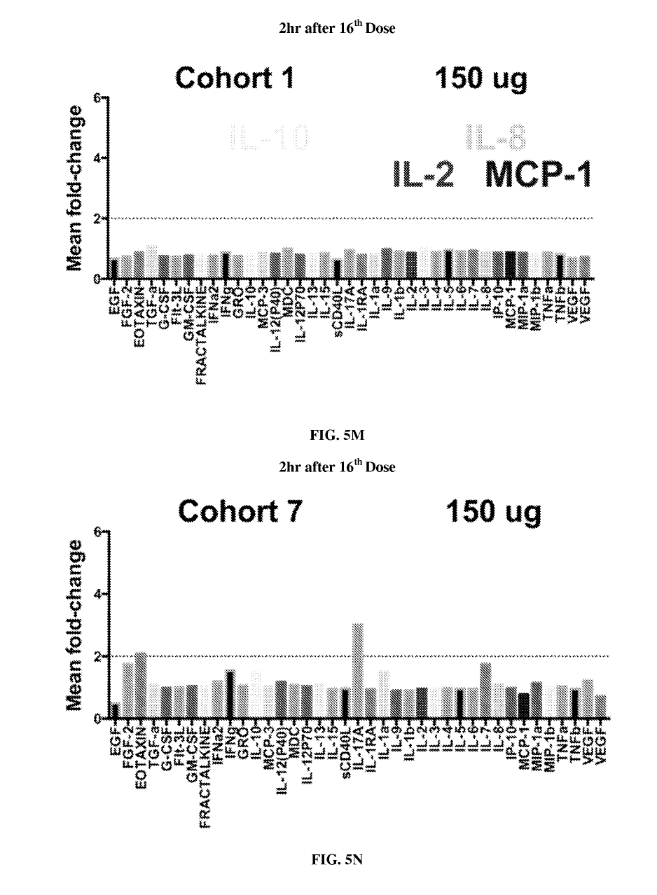

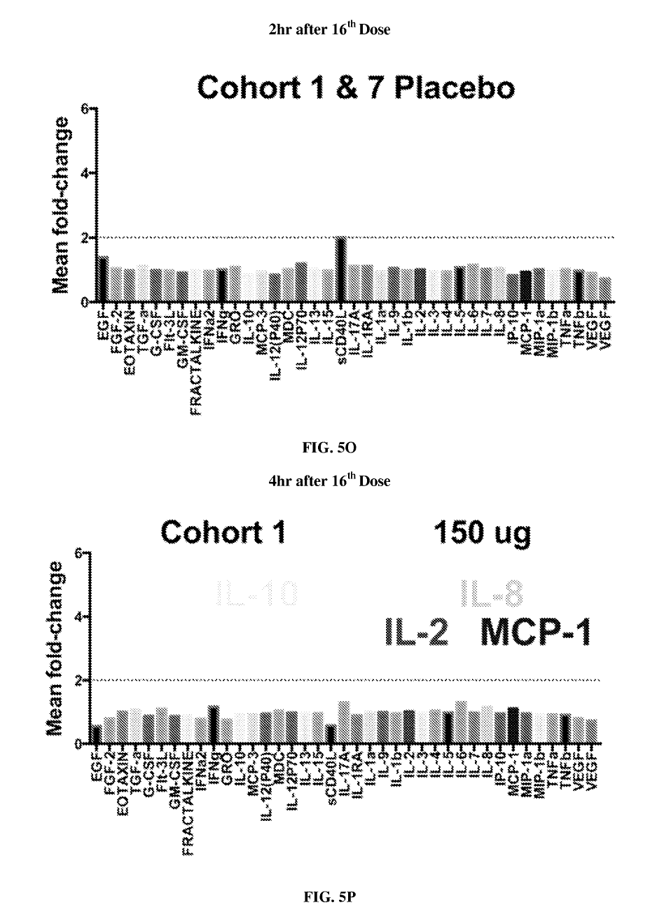

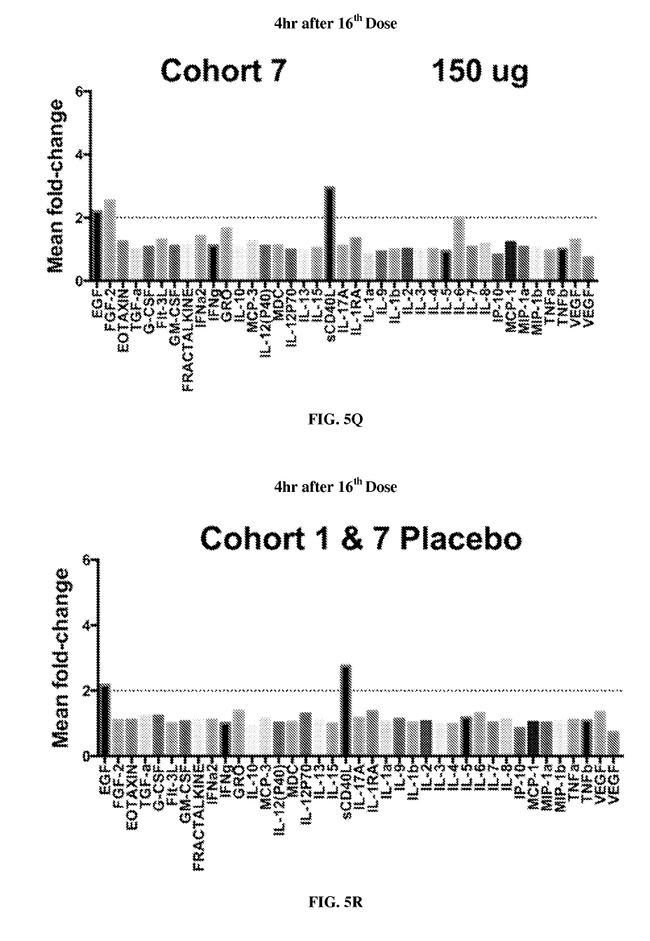

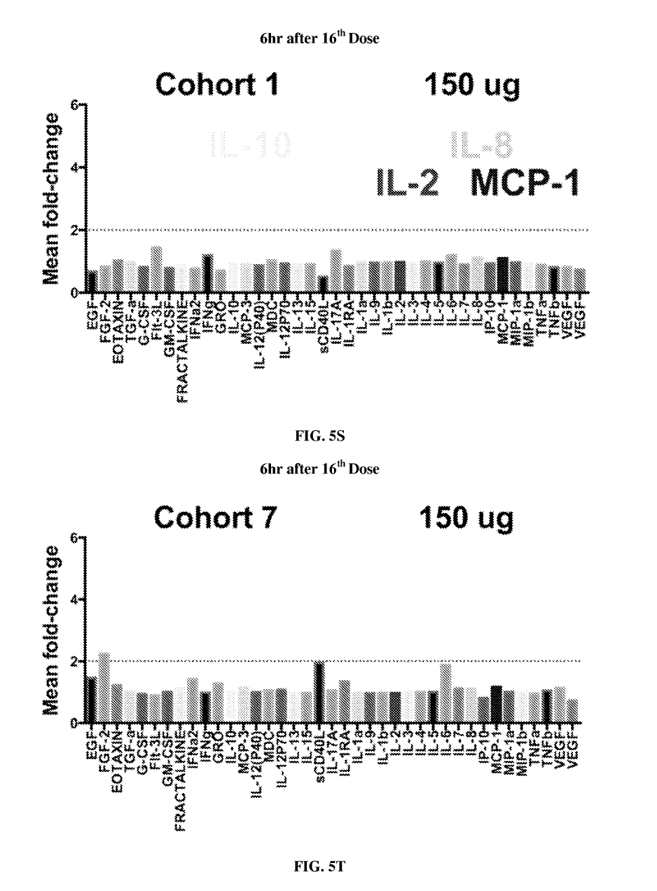

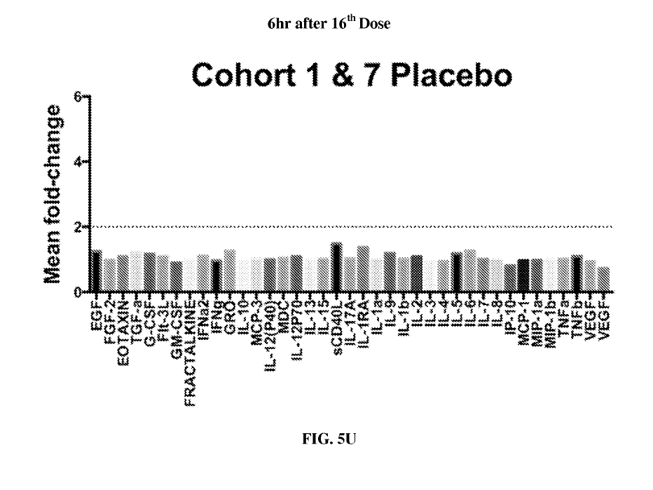

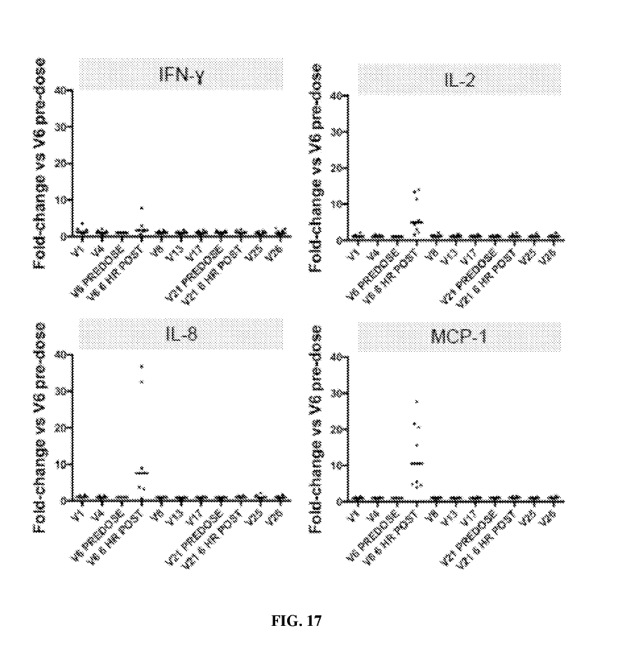

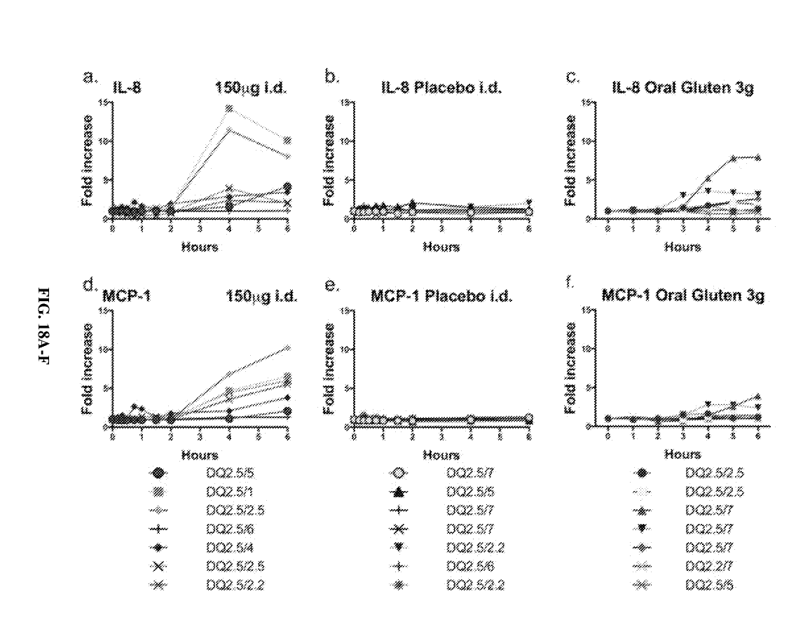

This is further supported by data showing that cytokine and chemokine levels were not elevated after treatment with the peptide composition in a twice-weekly dose regimen for 8 weeks compared to the first dose of the peptide composition, where certain cytokine and chemokine levels were elevated (compare FIGS. 5M-5U with FIGS. 5A-5L). Levels in subjects treated with placebo were not elevated after the first or last dose. Further data showed that unlike after the first dose (visit 6) was administered, after the last dose (visit 21) was adminsitered, IL-2, IL-8, and MCP-1 were not elevated (FIGS. 8-16). This was also the case for IFN-γ, IL-2, IL-8 and MCP-1 at times other than after the first dose (e.g., at visits 1, 4, 8, 13, 17, 25, and 26, FIG. 17). These results further indicate that circulating cytokines and chemokines may be used to identify a subject having Celiac disease after administration of a gluten peptide-containing composition because subjects having Celiac disease had elevated levels of cytokines and chemokines after an initial administration of the peptide composition but not after treatment with the peptide composition for several weeks.

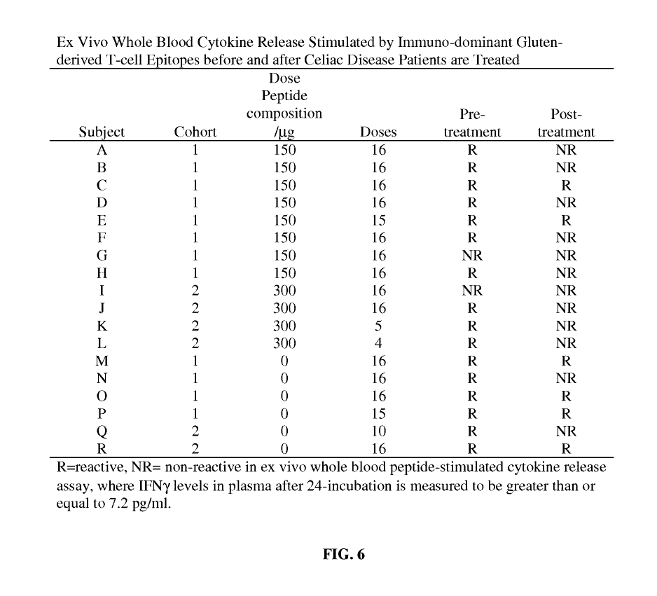

Reactivity of subjects having Celiac disease to the peptides was also assessed using an ex vivo whole blood cytokine release assay both pre- and post-treatment of the subjects with the peptide composition. Each subject was determined to be either reactive or non-reactive by incubating whole blood with the three peptides and measuring the levels of cytokines and chemokines released in the blood. A subject was determined to be reactive if IFN-γ levels in plasma after 24-hour incubation were greater than or equal to 7.2 pg/ml. Most subjects were determined to be reactive to the peptides pre-treatment and non-reactive to the peptides post-treatment (FIG. 6). It was also shown that levels of interleukin-2 and IFN-γ-inducible protein (IP-10) correlated with elevated concentrations of IFN-γ. These results suggest that an ex vivo whole blood cytokine release assay may also be used to identify a subject having Celiac disease, either alone or in combination with the levels of one or more circulating cytokines or chemokines described herein. Such methods are also provided herein.

In the pre-treatment blood sample collected 6 days after commencing 3-day oral gluten challenge, 7 of 8 subjects in Cohort 1 and 3 of 4 subjects in Cohort 2 who subsequently received at least 4 doses of the peptide composition were reactive to the peptide composition as judged by IFN-γ measured by ELISA. After patients were treated with the peptide composition, reactivity following gluten challenge was positive in only 2 of 7 subjects in Cohort 1 and 0 of 3 subjects in Cohort 2 who were previously reactive (FIG. 6). In contrast 5 of the 7 subjects in Cohorts 1 and 2 who received placebo were reactive after oral gluten challenge following treatment. IFN-γ plasma levels measured by MAGPIX correlated with levels assessed by ELISA. In addition, elevated IFN-γ levels were correlated with IL-2 and IP-10 when measured by MAGPIX. These results also suggest that an ex vivo whole blood cytokine release assay may also be used for identifying subjects that are likely to be responsive to a Celiac disease therapy such as the peptide composition and also for identifying subjects that have developed tolerance to gluten after therapy, either alone or in combination with the levels of one or more circulating cytokines or chemokines described herein.

TABLE 2
Plasma cytokine and chemokine concentrations (pg/mL) immediately before first
dose of peptide composition or placebo

| | Cohort | | | | | | | | | | |
|---|---|---|---|---|---|---|---|---|---|---|---|
| | 1 | 1 | 1 | 1 | 1 | 1 | 1 | 1 | 2 | 2 | 2 |
| | Dose | | | | | | | | | | |
| | 150 | 150 | 150 | 150 | 150 | 150 | 150 | 150 | 300 | 300 | 300 |
| EGF | 20 | 22 | 31 | 199 | 26 | 39 | 28 | 60 | 9 | 36 | 51 |
| FGF-2 | 32 | 35 | 69 | 239 | 23 | 91 | 77 | 73 | 29 | 114 | 101 |
| EOTAXIN | 14 | 45 | 53 | 73 | 102 | 73 | 37 | 32 | 40 | 62 | 57 |
| TGF-a | 3 | 3 | 3 | 380 | 3 | 3 | 3 | 3 | 5 | 3 | 4 |
| G-CSF | 11 | 34 | 34 | 123 | 19 | 29 | 29 | 34 | 14 | 46 | 91 |
| Flt-3L | 3 | 3 | 11 | 64 | 3 | 10 | 3 | 3 | 18 | 3 | 7 |
| GM-CSF | 10 | 12 | 8 | 95 | 9 | 18 | 3 | 20 | 3 | 14 | 37 |
| FRACTALKINE | 52 | 54 | 47 | 180 | 21 | 61 | 21 | 26 | 39 | 91 | 94 |
| IFNa2 | 13 | 50 | 19 | 293 | 9 | 30 | 11 | 19 | 11 | 41 | 131 |
| IFNg | 7 | 19 | 16 | 670 | 3 | 13 | 6 | 10 | 3 | 11 | 50 |
| GRO | 376 | 346 | 273 | 610 | 378 | 1075 | 1001 | 526 | 670 | 883 | 669 |
| IL-10 | 3 | 7 | 5 | 24 | 3 | 6 | 3 | 6 | 9 | 26 | 8 |
| MCP-3 | 42 | 71 | 11 | 325 | 3 | 26 | 11 | 10 | 24 | 41 | 118 |
| IL-12(P40) | 16 | 38 | 46 | 106 | 3 | 24 | 3 | 39 | 17 | 92 | 59 |
| MDC | 1169 | 760 | 1143 | 1900 | 1182 | 1325 | 568 | 804 | 576 | 939 | 941 |
| IL-12P70 | 3 | 5 | 3 | 86 | 3 | 7 | 4 | 5 | 13 | 7 | 9 |
| IL-13 | 11 | 28 | 107 | 107 | 3 | 3 | 3 | 3 | 3 | 30 | 45 |
| IL-15 | 3 | 7 | 5 | 12 | 3 | 6 | 3 | 4 | 4 | 17 | 4 |
| sCD40L | 2532 | 1742 | 1664 | 2079 | 2761 | 10000 | 10000 | 5538 | 1272 | 5031 | 2497 |
| IL-17A | 3 | 3 | 9 | 177 | 3 | 143 | 3 | 18 | 3 | 4 | 18 |
| IL-1RA | 45 | 47 | 45 | 571 | 16 | 52 | 25 | 42 | 21 | 68 | 121 |
| IL-1a | 10 | 17 | 15 | 710 | 3 | 28 | 3 | 6 | 3 | 73 | 60 |
| IL-9 | 3 | 3 | 3 | 8 | 3 | 3 | 3 | 3 | 3 | 6 | 3 |
| IL-1b | 3 | 4 | 3 | 18 | 3 | 3 | 3 | 3 | 3 | 12 | 6 |
| IL-2 | 3 | 6 | 4 | 14 | 3 | 3 | 3 | 4 | 3 | 14 | 5 |
| IL-3 | 3 | 3 | 3 | 3 | 3 | 3 | 3 | 3 | 3 | 4 | 3 |
| IL-4 | 3 | 30 | 6 | 106 | 3 | 3 | 3 | 4 | 7 | 27 | 39 |
| IL-5 | 3 | 5 | 3 | 27 | 3 | 3 | 3 | 3 | 3 | 3 | 5 |
| IL-6 | 3 | 7 | 6 | 23 | 3 | 3 | 3 | 3 | 3 | 14 | 5 |
| IL-7 | 3 | 3 | 3 | 10 | 3 | 5 | 3 | 3 | 3 | 3 | 7 |
| IL-8 | 8 | 14 | 10 | 162 | 3 | 115 | 3 | 11 | 5 | 16 | 39 |
| IP-10 | 351 | 361 | 366 | 449 | 418 | 516 | 514 | 282 | 253 | 346 | 329 |
| MCP-1 | 313 | 115 | 71 | 174 | 188 | 292 | 256 | 146 | 197 | 235 | 155 |
| MIP-1a | 5 | 5 | 9 | 20 | 3 | 3 | 3 | 3 | 3 | 5 | 8 |
| MIP-1b | 24 | 27 | 32 | 184 | 13 | 32 | 17 | 17 | 8 | 24 | 58 |
| TNFa | 7 | 7 | 6 | 20 | 8 | 16 | 8 | 7 | 5 | 8 | 12 |
| TNFb | 29 | 53 | 7 | 423 | 3 | 7 | 3 | 4 | 5 | 56 | 134 |
| VEGF | 46 | 25 | 211 | 1487 | 5 | 72 | 61 | 93 | 65 | 156 | 250 |

The second-to-last column is Mean Predose:Peptide composition treated.
The last column is SD Predose:Peptide composition treated.

| | Cohort | | | | | | | | | | | | | | | |
|---|---|---|---|---|---|---|---|---|---|---|---|---|---|---|---|---|
| | 2 | 2 | 2 | 2 | 2 | 2 | 2 | 7 | 7 | 7 | 7 | 7 | 7 | 7 | | |
| | Dose | | | | | | | | | | | | | | | |
| | 300 | 300 | 300 | 300 | 300 | 300 | 300 | 150 | 150 | 150 | 150 | 150 | 150 | 150 | | |
| EGF | 55 | 15 | 32 | 25 | 176 | 36 | 19 | 5 | 30 | 6 | 11 | 36 | 41 | 143 | 46 | 50 |
| FGF-2 | 22 | 103 | 146 | 80 | 500 | 84 | 100 | 40 | 33 | 63 | 46 | 93 | 46 | 238 | 99 | 101 |
| EOTAXIN | 41 | 78 | 60 | 55 | 188 | 88 | 54 | 49 | 46 | 76 | 39 | 52 | 31 | 38 | 59 | 33 |
| TGF-a | 3 | 5 | 6 | 6 | 17 | 17 | 4 | 3 | 3 | 3 | 3 | 6 | 3 | 3 | 20 | 75 |
| G-CSF | 16 | 159 | 131 | 128 | 172 | 67 | 98 | 26 | 20 | 57 | 63 | 66 | 128 | 85 | 67 | 49 |
| Flt-3L | 3 | 42 | 34 | 30 | 222 | 19 | 16 | 3 | 3 | 3 | 4 | 21 | 33 | 13 | 23 | 44 |
| GM-CSF | 4 | 28 | 53 | 38 | 436 | 51 | 27 | 5 | 13 | 17 | 19 | 29 | 21 | 47 | 41 | 85 |
| FRACTALKINE | 17 | 90 | 133 | 104 | 274 | 86 | 120 | 39 | 32 | 46 | 74 | 98 | 35 | 74 | 75 | 57 |
| IFNa2 | 13 | 122 | 207 | 54 | 145 | 88 | 49 | 13 | 15 | 24 | 24 | 56 | 16 | 188 | 66 | 75 |
| IFNg | 9 | 30 | 63 | 39 | 546 | 147 | 53 | 5 | 5 | 27 | 6 | 22 | 224 | 187 | 87 | 168 |
| GRO | 722 | 219 | 256 | 181 | 251 | 331 | 239 | 223 | 350 | 225 | 228 | 160 | 731 | 850 | 471 | 279 |
| IL-10 | 3 | 50 | 51 | 36 | 175 | 15 | 9 | 3 | 3 | 6 | 8 | 15 | 3 | 33 | 20 | 35 |
| MCP-3 | 9 | 104 | 94 | 78 | 43 | 72 | 29 | 9 | 9 | 9 | 14 | 42 | 10 | 39 | 50 | 66 |
| IL-12(P40) | 6 | 175 | 189 | 142 | 108 | 68 | 46 | 11 | 11 | 49 | 76 | 99 | 3 | 100 | 61 | 54 |
| MDC | 1085 | 1125 | 1043 | 1329 | 2539 | 1776 | 1174 | 121 | 641 | 799 | 1025 | 1595 | 1996 | 1287 | 1154 | 516 |
| IL-12P70 | 3 | 23 | 27 | 17 | 324 | 13 | 22 | 3 | 3 | 4 | 8 | 16 | 3 | 86 | 28 | 66 |
| IL-13 | 3 | 74 | 53 | 32 | 95 | 51 | 13 | 3 | 3 | 7 | 9 | 18 | 3 | 44 | 30 | 34 |
| IL-15 | 3 | 32 | 43 | 21 | 193 | 13 | 6 | 3 | 3 | 5 | 5 | 15 | 3 | 26 | 18 | 38 |
| sCD40L | 10000 | 2496 | 4340 | 813 | 2985 | 1395 | 2713 | 2122 | 3237 | 663 | 906 | 2316 | 6820 | 2673 | 3544 | 2838 |
| IL-17A | 3 | 16 | 10 | 8 | 254 | 107 | 29 | 3 | 3 | 10 | 3 | 8 | 64 | 73 | 39 | 65 |
| IL-1RA | 20 | 186 | 332 | 187 | 386 | 191 | 91 | 35 | 24 | 52 | 52 | 112 | 34 | 103 | 114 | 135 |
| IL-1a | 3 | 166 | 165 | 127 | 748 | 48 | 26 | 3 | 5 | 13 | 13 | 35 | 3 | 321 | 104 | 202 |
| IL-9 | 3 | 14 | 18 | 12 | 34 | 5 | 3 | 3 | 3 | 3 | 3 | 6 | 3 | 20 | 7 | 7 |
| IL-1b | 3 | 31 | 29 | 18 | 61 | 10 | 34 | 3 | 3 | 4 | 6 | 19 | 3 | 19 | 12 | 14 |
| IL-2 | 3 | 30 | 33 | 24 | 180 | 11 | 5 | 3 | 3 | 5 | 8 | 15 | 3 | 29 | 17 | 35 |
| IL-3 | 3 | 13 | 11 | 3 | 9 | 4 | 3 | 3 | 3 | 3 | 3 | 5 | 3 | 8 | 4 | 3 |
| IL-4 | 3 | 95 | 105 | 61 | 53 | 53 | 6 | 3 | 3 | 12 | 19 | 39 | 3 | 36 | 29 | 33 |
| IL-5 | 3 | 6 | 13 | 7 | 41 | 5 | 5 | 3 | 3 | 3 | 3 | 3 | 3 | 11 | 7 | 9 |
| IL-6 | 3 | 34 | 28 | 19 | 188 | 10 | 17 | 3 | 3 | 4 | 9 | 12 | 3 | 42 | 18 | 37 |
| IL-7 | 3 | 6 | 7 | 6 | 20 | 4 | 7 | 3 | 3 | 3 | 4 | 7 | 3 | 39 | 7 | 8 |
| IL-8 | 4 | 24 | 22 | 16 | 117 | 44 | 16 | 3 | 3 | 8 | 3 | 10 | 25 | 37 | 29 | 41 |
| IP-10 | 141 | 884 | 900 | 260 | 626 | 335 | 716 | 324 | 903 | 566 | 221 | 517 | 358 | 401 | 454 | 211 |
| MCP-1 | 190 | 310 | 277 | 109 | 203 | 159 | 262 | 103 | 147 | 268 | 170 | 186 | 165 | 213 | 195 | 67 |
| MIP-1a | 3 | 15 | 11 | 13 | 78 | 10 | 3 | 3 | 3 | 4 | 3 | 5 | 4 | 17 | 10 | 15 |
| MIP-1b | 11 | 38 | 60 | 36 | 426 | 81 | 53 | 16 | 13 | 18 | 19 | 34 | 14 | 134 | 55 | 87 |
| TNFa | 6 | 17 | 20 | 13 | 77 | 17 | 14 | 9 | 5 | 11 | 6 | 16 | 6 | 27 | 14 | 14 |
| TNFb | 3 | 186 | 266 | 114 | 78 | 124 | 12 | 3 | 3 | 8 | 9 | 31 | 3 | 43 | 64 | 101 |
| VEGF | 84 | 241 | 433 | 667 | 1922 | 295 | 268 | 35 | 40 | 144 | 102 | 220 | 1094 | 915 | 357 | 493 |

The second-to-last column is Mean Predose:Peptide composition treated.
The last column is SD Predose:Peptide composition treated.

| | Cohort | | | | | | | | | | | | | | | |
|---|---|---|---|---|---|---|---|---|---|---|---|---|---|---|---|---|
| | 2 | 2 | 2 | 2 | 2 | 2 | 2 | 7 | 7 | 7 | 7 | 7 | 7 | 7 | | |
| | Dose | | | | | | | | | | | | | | | |
| | 300 | 300 | 300 | 300 | 300 | 300 | 300 | 150 | 150 | 150 | 150 | 150 | 150 | 150 | | |
| EGF | 55 | 15 | 32 | 25 | 176 | 36 | 19 | 5 | 30 | 6 | 11 | 36 | 41 | 143 | 46 | 50 |
| FGF-2 | 22 | 103 | 146 | 80 | 500 | 84 | 100 | 40 | 33 | 63 | 46 | 93 | 46 | 238 | 99 | 101 |
| EOTAXIN | 41 | 78 | 60 | 55 | 188 | 88 | 54 | 49 | 46 | 76 | 39 | 52 | 31 | 38 | 59 | 33 |
| TGF-a | 3 | 5 | 6 | 6 | 17 | 17 | 4 | 3 | 3 | 3 | 3 | 6 | 3 | 3 | 20 | 75 |
| G-CSF | 16 | 159 | 131 | 128 | 172 | 67 | 98 | 26 | 20 | 57 | 63 | 66 | 128 | 85 | 67 | 49 |
| Flt-3L | 3 | 42 | 34 | 30 | 222 | 19 | 16 | 3 | 3 | 3 | 4 | 21 | 33 | 13 | 23 | 44 |
| GM-CSF | 4 | 28 | 53 | 38 | 436 | 51 | 27 | 5 | 13 | 17 | 19 | 29 | 21 | 47 | 41 | 85 |
| FRACTALKINE | 17 | 90 | 133 | 104 | 274 | 86 | 120 | 39 | 32 | 46 | 74 | 98 | 35 | 74 | 75 | 57 |
| IFNa2 | 13 | 122 | 207 | 54 | 145 | 88 | 49 | 13 | 15 | 24 | 24 | 56 | 16 | 188 | 66 | 75 |
| IFNg | 9 | 30 | 63 | 39 | 546 | 147 | 53 | 5 | 5 | 27 | 6 | 22 | 224 | 187 | 87 | 168 |
| GRO | 722 | 219 | 256 | 181 | 251 | 331 | 239 | 223 | 350 | 225 | 228 | 160 | 731 | 850 | 471 | 279 |
| IL-10 | 3 | 50 | 51 | 36 | 175 | 15 | 9 | 3 | 3 | 6 | 8 | 15 | 3 | 33 | 20 | 35 |
| MCP-3 | 9 | 104 | 94 | 78 | 43 | 72 | 29 | 9 | 9 | 9 | 14 | 42 | 10 | 39 | 50 | 66 |
| IL-12(P40) | 6 | 175 | 189 | 142 | 108 | 68 | 46 | 11 | 11 | 49 | 76 | 99 | 3 | 100 | 61 | 54 |
| MDC | 1085 | 1125 | 1043 | 1329 | 2539 | 1776 | 1174 | 121 | 641 | 799 | 1025 | 1595 | 1996 | 1287 | 1154 | 516 |
| IL-12P70 | 3 | 23 | 27 | 17 | 324 | 13 | 22 | 3 | 3 | 4 | 8 | 16 | 3 | 86 | 28 | 66 |
| IL-13 | 3 | 74 | 53 | 32 | 95 | 51 | 13 | 3 | 3 | 7 | 9 | 18 | 3 | 44 | 30 | 34 |
| IL-15 | 3 | 32 | 43 | 21 | 193 | 13 | 6 | 3 | 3 | 5 | 5 | 15 | 3 | 26 | 18 | 38 |
| sCD40L | 10000 | 2496 | 4340 | 813 | 2985 | 1395 | 2713 | 2122 | 3237 | 663 | 906 | 2316 | 6820 | 2673 | 3544 | 2838 |
| IL-17A | 3 | 16 | 10 | 8 | 254 | 107 | 29 | 3 | 3 | 10 | 3 | 8 | 64 | 73 | 39 | 65 |
| IL-1RA | 20 | 186 | 332 | 187 | 386 | 191 | 91 | 35 | 24 | 52 | 52 | 112 | 34 | 103 | 114 | 135 |
| IL-1a | 3 | 166 | 165 | 127 | 748 | 48 | 26 | 3 | 5 | 13 | 13 | 35 | 3 | 321 | 104 | 202 |
| IL-9 | 3 | 14 | 18 | 12 | 34 | 5 | 3 | 3 | 3 | 3 | 3 | 6 | 3 | 20 | 7 | 7 |
| IL-1b | 3 | 31 | 29 | 18 | 61 | 10 | 34 | 3 | 3 | 4 | 6 | 19 | 3 | 19 | 12 | 14 |
| IL-2 | 3 | 30 | 33 | 24 | 180 | 11 | 5 | 3 | 3 | 5 | 8 | 15 | 3 | 29 | 17 | 35 |
| IL-3 | 3 | 13 | 11 | 3 | 9 | 4 | 3 | 3 | 3 | 3 | 3 | 5 | 3 | 8 | 4 | 3 |
| IL-4 | 3 | 95 | 105 | 61 | 53 | 53 | 6 | 3 | 3 | 12 | 19 | 39 | 3 | 36 | 29 | 33 |
| IL-5 | 3 | 6 | 13 | 7 | 41 | 5 | 5 | 3 | 3 | 3 | 3 | 3 | 3 | 11 | 7 | 9 |
| IL-6 | 3 | 34 | 28 | 19 | 188 | 10 | 17 | 3 | 3 | 4 | 9 | 12 | 3 | 42 | 18 | 37 |
| IL-7 | 3 | 6 | 7 | 6 | 20 | 4 | 7 | 3 | 3 | 3 | 4 | 7 | 3 | 39 | 7 | 8 |
| IL-8 | 4 | 24 | 22 | 16 | 117 | 44 | 16 | 3 | 3 | 8 | 3 | 10 | 25 | 37 | 29 | 41 |
| IP-10 | 141 | 884 | 900 | 260 | 626 | 335 | 716 | 324 | 903 | 566 | 221 | 517 | 358 | 401 | 454 | 211 |
| MCP-1 | 190 | 310 | 277 | 109 | 203 | 159 | 262 | 103 | 147 | 268 | 170 | 186 | 165 | 213 | 195 | 67 |
| MIP-1a | 3 | 15 | 11 | 13 | 78 | 10 | 3 | 3 | 3 | 4 | 3 | 5 | 4 | 17 | 10 | 15 |
| MIP-1b | 11 | 38 | 60 | 36 | 426 | 81 | 53 | 16 | 13 | 18 | 19 | 34 | 14 | 134 | 55 | 87 |
| TNFa | 6 | 17 | 20 | 13 | 77 | 17 | 14 | 9 | 5 | 11 | 6 | 16 | 6 | 27 | 14 | 14 |
| TNFb | 3 | 186 | 266 | 114 | 78 | 124 | 12 | 3 | 3 | 8 | 9 | 31 | 3 | 43 | 64 | 101 |
| VEGF | 84 | 241 | 433 | 667 | 1922 | 295 | 268 | 35 | 40 | 144 | 102 | 220 | 1094 | 915 | 357 | 493 |

The second-to-last column is Mean Predose:Peptide composition treated.
The last column is SD Predose:Peptide composition treated.

| | Cohort | | | | | | | | | | | | | | | |
|---|---|---|---|---|---|---|---|---|---|---|---|---|---|---|---|---|
| | 1 | 1 | 1 | 1 | 2 | 2 | 2 | 7 | 7 | 7 | 7 | 7 | 7 | 7 | | |
| | Dose | | | | | | | | | | | | | | | |
| | 0 | 0 | 0 | 0 | 0 | 0 | 0 | 0 | 0 | 0 | 0 | 0 | 0 | 0 | | |
| EGF | 3 | 22 | 27 | 66 | 21 | 78 | 21 | 39 | 41 | 69 | 222 | 68 | 50 | 92 | 59 | 54 |
| FGF-2 | 38 | 40 | 56 | 75 | 110 | 262 | 71 | 119 | 80 | 80 | ND | 30 | 18 | 406 | 106 | 109 |
| EOTAXIN | 58 | 50 | 90 | 70 | 41 | 74 | 83 | 77 | 52 | 43 | 153 | 94 | 45 | 92 | 73 | 30 |
| TGF-a | 3 | 3 | 3 | 3 | 3 | 43 | 3 | 9 | 3 | 3 | 7 | 3 | 3 | 52 | 10 | 16 |
| G-CSF | 5 | 13 | 42 | 66 | 79 | 146 | 34 | 102 | 41 | 50 | 124 | 27 | 11 | 63 | 57 | 43 |
| Flt-3L | 3 | 3 | 3 | 4 | 3 | 67 | 87 | 62 | 3 | 3 | 18 | 3 | 3 | 43 | 22 | 30 |
| GM-CSF | 5 | 5 | 17 | 14 | 42 | 102 | 11 | 39 | 15 | 16 | ND | 10 | 3 | 53 | 26 | 28 |
| FRACTALKINE | 24 | 61 | 45 | 59 | 85 | 162 | 80 | 197 | 52 | 47 | 307 | 48 | 19 | 134 | 94 | 80 |
| IFNa2 | 13 | 11 | 16 | 29 | 38 | 347 | 36 | 134 | 29 | 24 | 138 | 19 | 6 | 81 | 66 | 92 |
| IFNg | 4 | 6 | 4 | 8 | 29 | 215 | 13 | 41 | 15 | 5 | 487 | 3 | 3 | 1086 | 137 | 304 |
| GRO | 523 | 703 | 397 | 735 | 169 | 555 | 121 | 181 | 562 | 440 | 376 | 779 | 661 | 1090 | 521 | 268 |
| IL-10 | 3 | 3 | 11 | 6 | 19 | 56 | 4 | 37 | 18 | 5 | 13 | 3 | 3 | 14 | 14 | 15 |
| MCP-3 | 5 | 7 | 15 | 29 | 24 | 177 | 17 | 45 | 22 | 3 | 60 | 13 | 3 | 288 | 51 | 82 |
| IL-12(P40) | 20 | 12 | 53 | 21 | 74 | 233 | 12 | 97 | 76 | 3 | 173 | 3 | 3 | 253 | 74 | 86 |
| MDC | 950 | 786 | 810 | 1117 | 898 | 889 | 862 | 878 | 1066 | 1070 | 1189 | 1041 | 709 | 3400 | 1119 | 671 |
| IL-12P70 | 3 | 3 | 6 | 7 | 21 | 172 | 10 | 61 | 8 | 6 | 753 | 4 | 3 | 65 | 80 | 199 |
| IL-13 | 3 | 3 | 6 | 5 | 19 | 248 | 3 | 25 | 8 | 3 | 266 | 3 | 4 | 129 | 52 | 93 |
| IL-15 | 3 | 3 | 6 | 3 | 11 | 50 | 3 | 18 | 13 | 3 | 15 | 3 | 3 | 21 | 11 | 13 |
| sCD40L | 1126 | 7270 | 9391 | 10000 | 1298 | 10000 | 237 | 2268 | 10000 | 10000 | 10000 | 8329 | 10000 | 4135 | 6718 | 3959 |
| IL-17A | 3 | 3 | 3 | 5 | 19 | 52 | 9 | 19 | 8 | 3 | 167 | 3 | 3 | 334 | 45 | 94 |
| IL-1RA | 22 | 24 | 51 | 58 | 102 | 659 | 60 | 197 | 96 | 52 | 807 | 33 | 14 | 295 | 176 | 250 |
| IL-1a | 10 | 3 | 12 | 5 | 16 | 165 | 5 | 54 | 12 | 6 | 116 | 9 | 3 | 95 | 36 | 52 |
| IL-9 | 3 | 3 | 3 | 3 | 4 | 24 | 3 | 6 | 3 | 3 | 16 | 3 | 3 | 8 | 6 | 6 |
| IL-1b | 3 | 3 | 4 | 3 | 5 | 34 | 3 | 19 | 8 | 3 | 156 | 3 | 3 | 9 | 18 | 41 |
| IL-2 | 3 | 3 | 5 | 3 | 7 | 40 | 3 | 18 | 8 | 3 | 27 | 3 | 3 | 14 | 10 | 11 |
| IL-3 | 3 | 3 | 3 | 3 | 3 | 15 | 3 | 12 | 4 | 3 | 3 | 3 | 3 | 4 | 5 | 4 |
| IL-4 | 3 | 3 | 15 | 4 | 20 | 164 | 3 | 69 | 25 | 3 | 28 | 3 | 3 | 178 | 37 | 60 |
| IL-5 | 3 | 3 | 3 | 3 | 5 | 33 | 3 | 6 | 3 | 3 | 27 | 3 | 3 | 17 | 8 | 10 |
| IL-6 | 3 | 3 | 6 | 3 | 13 | 48 | 3 | 30 | 9 | 3 | 79 | 3 | 3 | 59 | 19 | 25 |
| IL-7 | 3 | 3 | 3 | 3 | 7 | 15 | 7 | 21 | 3 | 3 | 96 | 3 | 3 | 10 | 13 | 25 |
| IL-8 | 4 | 3 | 3 | 9 | 13 | 74 | 8 | 7 | 9 | 3 | 80 | 4 | 3 | 292 | 37 | 78 |
| IP-10 | 672 | 259 | 366 | 296 | 1155 | 549 | 478 | 223 | 364 | 769 | 730 | 340 | 175 | 352 | 480 | 270 |
| MCP-1 | 35 | 106 | 220 | 107 | 222 | 177 | 313 | 212 | 186 | 229 | 162 | 227 | 159 | 238 | 185 | 69 |
| MIP-1a | 3 | 3 | 3 | 3 | 9 | 20 | 3 | 9 | 6 | 4 | 43 | 3 | 3 | 95 | 15 | 26 |
| MIP-1b | 28 | 4 | 15 | 16 | 50 | 132 | 18 | 49 | 23 | 31 | 213 | 17 | 12 | 573 | 84 | 152 |
| TNFa | 6 | 3 | 10 | 10 | 27 | 27 | 11 | 13 | 4 | 17 | 49 | 6 | 6 | 165 | 25 | 42 |
| TNFb | 3 | 3 | 12 | 16 | 12 | 463 | 3 | 21 | 21 | 3 | 164 | 3 | 3 | 2015 | 196 | 538 |
| VEGF | 27 | 109 | 91 | 124 | 287 | 817 | 82 | 231 | 116 | 60 | 1209 | 60 | 109 | 1596 | 351 | 493 |

The second-to-last column is Mean Predose:Placebo treated.
The last column is SD Predose:Placebo treated.

TABLE 3
Plasma cytokine and chemokine fold-change at 2 h post dose peptide composition or
placebo compared to pre-dose

Cohort

| | | | | | | | |
|---|---|---|---|---|---|---|---|
| 1 | 1 | 1 | 1 | 1 | 1 | 1 | 1 |

Dose

| | 150 | 150 | 150 | 150 | 150 | 150 | 150 | 150 | | |
|---|---|---|---|---|---|---|---|---|---|---|
| EGF | 2.8 | 1.2 | 1.3 | 2.6 | 0.5 | 0.9 | 0.8 | 2.3 | 1.6 | 0.9 |
| FGF-2 | 0.9 | 1.3 | 1.1 | 1.2 | 1.5 | 1.0 | 1.0 | 1.1 | 1.1 | 0.2 |
| EOTAXIN | 1.0 | 1.2 | 1.2 | 1.1 | 1.3 | 1.0 | 0.9 | 1.0 | 1.1 | 0.1 |
| TGF-a | 1.0 | 1.0 | 1.0 | 16.2 | 1.0 | 1.0 | 1.0 | 1.0 | 2.9 | 5.4 |
| G-CSF | 1.3 | 1.2 | 1.0 | 1.1 | 1.2 | 0.9 | 1.0 | 0.9 | 1.1 | 0.2 |
| Flt-3L | 1.0 | 1.0 | 1.3 | 1.1 | 1.0 | 0.7 | 1.0 | 1.0 | 1.0 | 0.2 |
| GM-CSF | 1.1 | 2.1 | 2.8 | 1.3 | 5.6 | 1.3 | 1.8 | 1.3 | 2.2 | 1.5 |
| FRACTALKINE | 1.1 | 1.3 | 1.0 | 1.3 | 2.0 | 1.1 | 1.1 | 1.0 | 1.2 | 0.3 |
| IFNa2 | 1.1 | 1.3 | 1.2 | 1.0 | 2.4 | 1.2 | 1.2 | 1.1 | 1.3 | 0.5 |
| IFNg | 1.2 | 1.2 | 1.2 | 3.1 | 1.0 | 1.1 | 0.6 | 1.0 | 1.3 | 0.8 |
| GRO | 1.9 | 1.3 | 1.6 | 1.5 | 0.8 | 1.0 | 0.8 | 2.1 | 1.4 | 0.5 |
| IL-10 | 1.0 | 1.5 | 1.7 | 1.0 | 1.2 | 1.1 | 1.0 | 1.6 | 1.3 | 0.3 |
| MCP-3 | 1.0 | 1.1 | 1.2 | 1.5 | 1.0 | 1.1 | 1.0 | 1.0 | 1.1 | 0.2 |
| IL-12(P40) | 1.0 | 1.2 | 1.1 | 1.0 | 1.0 | 0.9 | 1.0 | 0.9 | 1.0 | 0.1 |
| MDC | 1.0 | 1.1 | 1.0 | 2.0 | 1.0 | 1.0 | 1.0 | 1.2 | 1.2 | 0.4 |
| IL-12P70 | 1.2 | 1.6 | 1.7 | 1.1 | 1.0 | 1.3 | 1.0 | 0.9 | 1.2 | 0.3 |
| IL-13 | 1.2 | 1.1 | 1.0 | 1.0 | 1.0 | 1.0 | 1.0 | 1.0 | 1.0 | 0.1 |
| IL-15 | 1.0 | 1.3 | 1.2 | 1.1 | 1.0 | 1.0 | 1.0 | 0.8 | 1.1 | 0.2 |
| sCD40L | 3.9 | 1.1 | 1.1 | 0.8 | 0.6 | 1.0 | 1.0 | 1.8 | 1.4 | 1.1 |
| IL-17A | 1.0 | 3.3 | 1.4 | 3.9 | 1.0 | 1.0 | 1.0 | 0.8 | 1.7 | 1.2 |
| IL-1RA | 1.0 | 1.5 | 1.2 | 1.1 | 2.2 | 1.1 | 1.1 | 1.1 | 1.3 | 0.4 |
| IL-1a | 1.0 | 1.2 | 1.2 | 1.4 | 1.0 | 1.1 | 1.0 | 0.9 | 1.1 | 0.2 |
| IL-9 | 1.0 | 1.0 | 1.0 | 1.0 | 1.0 | 1.0 | 1.0 | 1.0 | 1.0 | 0.0 |
| IL-1b | 1.0 | 1.5 | 1.1 | 1.1 | 1.0 | 1.0 | 1.0 | 1.0 | 1.1 | 0.2 |
| IL-2 | 1.0 | 4.6 | 5.4 | 1.2 | 19.9 | 10.3 | 1.0 | 3.2 | 5.8 | 6.5 |
| IL-3 | 1.0 | 1.0 | 1.0 | 1.0 | 1.0 | 1.0 | 1.0 | 1.0 | 1.0 | 0.0 |
| IL-4 | 2.8 | 1.2 | 1.3 | 1.0 | 1.0 | 1.0 | 1.0 | 1.0 | 1.3 | 0.6 |
| IL-5 | 1.0 | 1.2 | 1.0 | 1.1 | 1.0 | 1.0 | 1.0 | 1.0 | 1.0 | 0.1 |
| IL-6 | 1.0 | 2.0 | 1.6 | 1.2 | 1.0 | 1.0 | 1.0 | 1.4 | 1.3 | 0.4 |
| IL-7 | 1.0 | 1.6 | 1.0 | 1.1 | 1.0 | 1.1 | 1.0 | 1.3 | 1.1 | 0.2 |
| IL-8 | 1.1 | 3.3 | 2.9 | 2.6 | 25.0 | 1.2 | 2.0 | 4.0 | 5.3 | 8.0 |
| IP-10 | 1.0 | 1.3 | 1.0 | 1.2 | 1.4 | 1.0 | 1.2 | 0.8 | 1.1 | 0.2 |
| MCP-1 | 0.9 | 2.3 | 1.6 | 1.1 | 3.0 | 1.4 | 1.1 | 1.3 | 1.6 | 0.7 |
| MIP-1a | 1.1 | 2.1 | 1.3 | 1.4 | 9.9 | 1.4 | 1.0 | 1.3 | 2.4 | 3.0 |
| MIP-1b | 1.1 | 2.0 | 2.2 | 1.8 | 11.2 | 1.5 | 1.3 | 2.1 | 2.9 | 3.4 |
| TNFa | 1.1 | 3.1 | 2.7 | 1.3 | 5.0 | 1.6 | 1.4 | 2.2 | 2.3 | 1.3 |
| TNFb | 1.1 | 1.1 | 1.0 | 1.2 | 1.0 | 1.0 | 1.0 | 1.1 | 1.1 | 0.1 |
| VEGF | 1.2 | 3.4 | 1.0 | 1.8 | 6.2 | 1.0 | 0.8 | 1.1 | 2.1 | 1.9 |

The second-to-last column is Mean 2 hr Post dose:Cohort 1.
The last column is SD 2 hr Post dose:Cohort 1.

| | Cohort | | | | | | | | | | | |
|---|---|---|---|---|---|---|---|---|---|---|---|---|
| | 2 | 2 | 2 | 2 | 2 | 2 | 2 | 2 | 2 | 2 | | |
| | Dose | | | | | | | | | | | |
| | 300 | 300 | 300 | 300 | 300 | 300 | 300 | 300 | 300 | 300 | | |
| EGF | 0.3 | 0.8 | 0.8 | 0.9 | 0.9 | 1.8 | 1.8 | 0.3 | 2.0 | 2.3 | 1.2 | 0.7 |
| FGF-2 | 0.8 | 0.8 | 1.3 | 0.9 | 0.8 | 1.2 | 1.1 | 0.2 | 1.1 | 0.7 | 0.9 | 0.3 |
| EOTAXIN | 0.9 | 0.9 | 1.1 | 1.0 | 1.0 | 1.3 | 1.2 | 0.8 | 1.1 | 1.1 | 1.0 | 0.1 |
| TGF-a | 1.1 | 1.0 | 1.2 | 1.0 | 0.7 | 1.8 | 1.0 | 0.2 | 0.6 | 0.7 | 0.9 | 0.4 |
| G-CSF | 0.9 | 0.9 | 1.1 | 0.7 | 0.8 | 1.3 | 0.9 | 0.5 | 1.1 | 0.5 | 0.9 | 0.3 |
| Flt-3L | 0.9 | 1.0 | 3.2 | 1.0 | 0.5 | 1.5 | 0.8 | 0.1 | 0.9 | 0.2 | 1.0 | 0.9 |
| GM-CSF | 1.0 | 1.8 | 1.3 | 0.8 | 0.9 | 1.4 | 1.1 | 0.1 | 1.0 | 1.7 | 1.1 | 0.5 |
| FRACTALKINE | 1.0 | 1.0 | 1.2 | 0.6 | 0.8 | 1.2 | 1.0 | 0.3 | 0.8 | 0.5 | 0.8 | 0.3 |
| IFNa2 | 0.9 | 1.0 | 1.1 | 0.6 | 0.9 | 1.3 | 1.1 | 0.4 | 1.0 | 0.7 | 0.9 | 0.3 |
| IFNg | 1.0 | 0.9 | 1.3 | 1.2 | 0.8 | 1.4 | 1.1 | 0.1 | 0.6 | 0.2 | 0.9 | 0.4 |
| GRO | 0.3 | 0.7 | 0.5 | 1.0 | 1.0 | 1.4 | 1.4 | 2.2 | 2.0 | 2.7 | 1.3 | 0.8 |
| IL-10 | 0.9 | 1.0 | 1.2 | 1.0 | 0.9 | 1.0 | 1.0 | 0.1 | 1.0 | 1.0 | 0.9 | 0.3 |
| MCP-3 | 1.0 | 1.0 | 1.1 | 0.8 | 1.0 | 1.2 | 1.1 | 0.6 | 0.9 | 0.8 | 0.9 | 0.2 |
| IL-12(P40) | 1.2 | 0.9 | 1.1 | 0.6 | 0.9 | 1.3 | 1.0 | 0.7 | 1.0 | 0.9 | 1.0 | 0.2 |
| MDC | 0.8 | 0.9 | 1.1 | 1.0 | 0.9 | 0.8 | 1.0 | 0.4 | 1.0 | 0.7 | 0.9 | 0.2 |
| IL-12P70 | 1.1 | 1.0 | 1.2 | 1.0 | 0.6 | 1.4 | 1.1 | 0.1 | 0.9 | 0.4 | 0.9 | 0.4 |
| IL-13 | 1.0 | 1.0 | 1.1 | 1.0 | 0.9 | 1.2 | 1.2 | 0.2 | 0.8 | 0.6 | 0.9 | 0.3 |
| IL-15 | 0.9 | 0.9 | 1.2 | 1.0 | 0.9 | 1.1 | 1.0 | 0.1 | 0.9 | 0.9 | 0.9 | 0.3 |
| sCD40L | 0.4 | 0.8 | 0.3 | 1.0 | 1.2 | 2.3 | 4.8 | 1.2 | 7.2 | 3.7 | 2.3 | 2.3 |
| IL-17A | 1.0 | 1.2 | 1.5 | 1.0 | 0.7 | 1.3 | 1.0 | 0.1 | 0.7 | 0.4 | 0.9 | 0.4 |
| IL-1RA | 0.9 | 0.9 | 1.1 | 0.7 | 0.8 | 1.4 | 1.1 | 0.3 | 1.0 | 0.7 | 0.9 | 0.3 |
| IL-1a | 1.0 | 1.2 | 2.0 | 1.0 | 0.9 | 1.0 | 1.0 | 0.3 | 0.8 | 0.8 | 1.0 | 0.4 |
| IL-9 | 1.0 | 0.9 | 1.0 | 1.0 | 0.9 | 1.1 | 1.0 | 0.2 | 0.9 | 1.0 | 0.9 | 0.3 |
| IL-1b | 1.0 | 1.0 | 1.5 | 1.0 | 0.8 | 1.1 | 1.0 | 0.2 | 1.0 | 0.2 | 0.9 | 0.4 |
| IL-2 | 1.0 | 3.1 | 4.8 | 1.0 | 1.2 | 1.1 | 1.0 | 0.1 | 1.5 | 22.6 | 3.7 | 6.7 |
| IL-3 | 1.0 | 1.0 | 1.0 | 1.0 | 0.9 | 1.0 | 1.1 | 0.7 | 1.0 | 1.0 | 1.0 | 0.1 |
| IL-4 | 1.0 | 0.9 | 1.1 | 1.0 | 0.9 | 1.3 | 1.1 | 0.6 | 0.9 | 0.5 | 0.9 | 0.2 |
| IL-5 | 1.0 | 1.0 | 1.1 | 1.0 | 0.9 | 1.4 | 1.2 | 0.1 | 0.9 | 0.6 | 0.9 | 0.4 |
| IL-6 | 1.2 | 1.0 | 1.9 | 1.0 | 1.0 | 1.1 | 1.0 | 0.2 | 2.4 | 0.5 | 1.1 | 0.6 |
| IL-7 | 1.0 | 1.0 | 1.2 | 1.0 | 0.7 | 1.8 | 1.2 | 0.3 | 1.2 | 0.5 | 1.0 | 0.4 |
| IL-8 | 1.1 | 2.3 | 1.6 | 1.1 | 1.4 | 1.3 | 1.1 | 0.2 | 0.8 | 4.5 | 1.5 | 1.2 |
| IP-10 | 1.2 | 1.0 | 1.3 | 1.0 | 1.0 | 0.5 | 0.7 | 0.4 | 0.8 | 0.6 | 0.8 | 0.3 |
| MCP-1 | 1.1 | 1.2 | 1.4 | 0.9 | 1.1 | 1.1 | 1.1 | 1.1 | 1.1 | 2.3 | 1.3 | 0.4 |
| MIP-1a | 1.0 | 1.2 | 1.3 | 1.0 | 1.1 | 1.2 | 1.0 | 0.4 | 0.7 | 1.0 | 1.0 | 0.3 |
| MIP-1b | 0.7 | 1.5 | 1.4 | 0.9 | 1.2 | 1.5 | 1.0 | 0.3 | 0.6 | 1.3 | 1.0 | 0.4 |
| TNFa | 0.8 | 2.2 | 1.9 | 1.1 | 0.3 | 1.4 | 1.0 | 0.2 | 1.1 | 2.4 | 1.3 | 0.7 |
| TNFb | 0.9 | 0.9 | 1.1 | 1.0 | 0.9 | 1.5 | 1.2 | 0.4 | 0.9 | 0.6 | 0.9 | 0.3 |
| VEGF | 0.9 | 0.9 | 1.3 | 1.0 | 0.8 | 1.4 | 0.9 | 0.2 | 0.8 | 0.5 | 0.9 | 0.4 |

The second-to-last column is Mean 2 hr Post dose:Cohort 2.
The last column is SD 2 hr Post dose:Cohort 2.

| | Cohort | | | | | | | | |
|---|---|---|---|---|---|---|---|---|---|
| | 7 | 7 | 7 | 7 | 7 | 7 | 7 | | |
| | Dose | | | | | | | | |
| | 150 | 150 | 150 | 150 | 150 | 150 | 150 | | |
| EGF | 1.1 | 1.1 | 1.5 | 2.4 | 1.0 | 1.5 | 0.7 | 1.3 | 0.6 |
| FGF-2 | 0.5 | 0.9 | 0.9 | 1.4 | 0.9 | 1.0 | 0.7 | 0.9 | 0.3 |
| EOTAXIN | 1.0 | 1.8 | 1.1 | 1.0 | 1.5 | 1.3 | 0.8 | 1.1 | 0.2 |
| TGF-a | 1.0 | 1.0 | 1.0 | 1.0 | 0.9 | 1.0 | 1.0 | 1.0 | 0.0 |
| G-CSF | 0.8 | 1.1 | 0.9 | 1.1 | 0.9 | 0.7 | 0.8 | 0.9 | 0.2 |
| Flt-3L | 1.0 | 1.0 | 1.0 | 0.7 | 0.3 | 0.7 | 0.3 | 0.7 | 0.3 |
| GM-CSF | 0.7 | 1.0 | 1.1 | 1.1 | 0.9 | 0.9 | 0.6 | 0.9 | 0.2 |
| FRACTALKINE | 0.6 | 0.9 | 1.0 | 1.0 | 0.7 | 1.1 | 0.8 | 0.9 | 0.2 |
| IFNa2 | 0.5 | 0.9 | 1.0 | 1.3 | 0.8 | 1.0 | 0.8 | 0.9 | 0.3 |
| IFNg | 0.6 | 0.8 | 1.0 | 1.2 | 1.4 | 0.7 | 0.7 | 0.9 | 0.3 |
| GRO | 1.2 | 1.1 | 1.3 | 2.0 | 1.0 | 1.6 | 0.6 | 1.3 | 0.4 |
| IL-10 | 0.9 | 1.0 | 1.0 | 1.0 | 0.8 | 1.0 | 1.0 | 1.0 | 0.1 |
| MCP-3 | 0.3 | 0.9 | 1.2 | 1.1 | 0.9 | 1.0 | 0.7 | 0.9 | 0.3 |
| IL-12(P40) | 0.6 | 0.9 | 1.0 | 1.0 | 0.9 | 1.0 | 0.9 | 0.9 | 0.2 |
| MDC | 1.0 | 1.1 | 1.1 | 1.0 | 1.1 | 0.9 | 0.9 | 1.0 | 0.1 |
| IL-12P70 | 1.0 | 1.0 | 1.1 | 1.1 | 0.7 | 1.2 | 0.7 | 1.0 | 0.2 |
| IL-13 | 1.0 | 1.0 | 1.1 | 1.0 | 0.8 | 1.0 | 0.8 | 1.0 | 0.1 |
| IL-15 | 1.0 | 1.0 | 1.1 | 1.0 | 0.8 | 1.0 | 0.9 | 1.0 | 0.1 |
| sCD40L | 1.1 | 1.8 | 1.3 | 3.0 | 0.3 | 1.5 | 0.8 | 1.4 | 0.9 |
| IL-17A | 1.0 | 1.0 | 0.9 | 1.0 | 1.4 | 0.7 | 0.7 | 1.0 | 0.3 |
| IL-1RA | 0.5 | 1.0 | 1.0 | 1.2 | 0.8 | 1.1 | 0.8 | 0.9 | 0.2 |
| IL-1a | 1.0 | 0.8 | 0.9 | 1.0 | 0.9 | 1.0 | 0.7 | 0.9 | 0.1 |
| IL-9 | 1.0 | 1.0 | 1.0 | 1.0 | 0.8 | 1.0 | 0.7 | 0.9 | 0.1 |
| IL-1b | 1.0 | 1.0 | 1.1 | 1.0 | 0.9 | 1.0 | 0.8 | 1.0 | 0.1 |
| IL-2 | 1.0 | 1.0 | 2.3 | 1.0 | 0.9 | 1.0 | 0.8 | 1.2 | 0.5 |
| IL-3 | 1.0 | 1.0 | 1.0 | 1.0 | 0.8 | 1.0 | 0.9 | 1.0 | 0.1 |
| IL-4 | 1.0 | 1.0 | 1.0 | 0.9 | 0.8 | 1.0 | 1.0 | 1.0 | 0.1 |
| IL-5 | 1.0 | 1.0 | 1.0 | 1.0 | 1.0 | 1.0 | 0.8 | 1.0 | 0.1 |
| IL-6 | 1.0 | 1.0 | 1.4 | 1.5 | 1.0 | 1.0 | 0.8 | 1.1 | 0.3 |
| IL-7 | 1.0 | 1.0 | 1.0 | 1.1 | 0.6 | 1.0 | 0.9 | 0.9 | 0.2 |
| IL-8 | 1.0 | 1.3 | 1.4 | 1.0 | 1.9 | 0.8 | 0.7 | 1.2 | 0.4 |
| IP-10 | 0.9 | 0.8 | 0.8 | 0.8 | 0.6 | 0.5 | 0.8 | 0.7 | 0.1 |
| MCP-1 | 0.9 | 1.0 | 1.0 | 1.0 | 1.8 | 1.1 | 1.0 | 1.1 | 0.3 |
| MIP-1a | 1.0 | 1.0 | 1.2 | 1.0 | 0.9 | 0.8 | 0.8 | 1.0 | 0.2 |
| MIP-1b | 0.7 | 1.0 | 1.3 | 1.0 | 1.2 | 0.9 | 0.8 | 1.0 | 0.2 |
| TNFa | 0.8 | 1.1 | 1.1 | 1.0 | 0.9 | 1.0 | 0.9 | 1.0 | 0.1 |
| TNFb | 1.0 | 1.0 | 1.1 | 1.2 | 0.9 | 1.0 | 0.9 | 1.0 | 0.1 |
| VEGF | 0.5 | 0.8 | 1.0 | 1.1 | 1.1 | 0.9 | 0.7 | 0.8 | 0.2 |

The second-to-last column is Mean 2 hr Post dose:Cohort 7.
The last column is SD 2 hr Post dose:Cohort 7.

| | Cohort | | | | | | | | | | | | | | | |
|---|---|---|---|---|---|---|---|---|---|---|---|---|---|---|---|---|
| | 1 | 1 | 1 | 1 | 2 | 2 | 2 | 7 | 7 | 7 | 7 | 7 | 7 | 7 | | |
| | Dose | | | | | | | | | | | | | | | |
| | 0 | 0 | 0 | 0 | 0 | 0 | 0 | 0 | 0 | 0 | 0 | 0 | 0 | 0 | | |
| EGF | 1.1 | 1.4 | 2.6 | 0.7 | 0.7 | 0.9 | 1.0 | 0.9 | 0.5 | 1.8 | 0.9 | 0.2 | 0.5 | 0.7 | 1.0 | 0.6 |
| FGF-2 | 1.2 | 1.0 | 1.1 | 0.8 | 0.8 | 0.9 | 1.4 | 0.8 | 1.7 | 1.2 | ND | 0.9 | 1.0 | 0.9 | 1.1 | 0.3 |
| EOTAXIN | 1.2 | 1.0 | 1.0 | 1.0 | 1.0 | 0.9 | 1.0 | 0.9 | 0.9 | 0.9 | 0.7 | 1.1 | 1.0 | 0.7 | 1.0 | 0.1 |
| TGF-a | 1.0 | 1.0 | 1.0 | 1.0 | 1.0 | 1.0 | 1.0 | 0.7 | 1.0 | 1.0 | 0.5 | 1.0 | 1.0 | 0.8 | 0.9 | 0.2 |
| G-CSF | 1.4 | 1.2 | 1.1 | 0.9 | 0.9 | 0.9 | 1.1 | 0.9 | 1.0 | 1.1 | 0.6 | 0.8 | 0.8 | 0.8 | 1.0 | 0.2 |
| Flt-3L | 1.0 | 1.0 | 1.0 | 0.9 | 1.0 | 0.8 | 0.9 | 0.7 | 7.1 | 1.0 | 0.4 | 1.0 | 1.0 | 0.7 | 1.2 | 1.7 |
| GM-CSF | 1.4 | 1.1 | 1.1 | 0.9 | 0.7 | 0.9 | 1.4 | 0.9 | 1.5 | 1.1 | ND | 1.0 | 1.0 | 0.7 | 1.1 | 0.3 |
| FRACTALKINE | 1.3 | 0.8 | 1.0 | 0.8 | 0.9 | 0.9 | 1.3 | 0.8 | 1.4 | 1.0 | 0.6 | 0.8 | 1.3 | 0.8 | 1.0 | 0.3 |
| IFNa2 | 1.5 | 1.1 | 1.2 | 0.8 | 0.8 | 0.9 | 1.8 | 0.7 | 1.5 | 1.0 | 0.7 | 1.0 | 1.3 | 0.8 | 1.1 | 0.3 |
| IFNg | 0.9 | 0.9 | 1.3 | 0.9 | 0.6 | 0.9 | 1.1 | 0.9 | 3.0 | 1.0 | 1.0 | 1.2 | 2.4 | 0.8 | 1.2 | 0.7 |
| GRO | 1.6 | 1.2 | 1.4 | 0.9 | 0.9 | 1.1 | 1.2 | 0.7 | 0.3 | 2.2 | 1.4 | 1.1 | 0.5 | 0.6 | 1.0 | 0.4 |
| IL-10 | 1.0 | 1.0 | 0.9 | 0.8 | 1.0 | 1.0 | 1.8 | 0.8 | 1.0 | 1.0 | 0.8 | 1.0 | 1.0 | 0.7 | 1.0 | 0.3 |
| MCP-3 | 1.8 | 1.2 | 1.2 | 1.0 | 0.9 | 1.0 | 1.6 | 0.9 | 1.3 | 1.0 | 0.7 | 1.0 | 1.0 | 0.9 | 1.1 | 0.3 |
| IL-12(P40) | 1.1 | 0.8 | 1.0 | 0.7 | 0.9 | 1.0 | 2.1 | 1.0 | 1.0 | 1.0 | 0.6 | 1.0 | 1.0 | 0.9 | 1.0 | 0.3 |
| MDC | 0.9 | 0.9 | 1.1 | 0.8 | 1.1 | 0.9 | 1.1 | 0.9 | 1.2 | 0.9 | 0.8 | 0.9 | 1.0 | 0.8 | 1.0 | 0.1 |
| IL-12P70 | 1.0 | 1.0 | 1.0 | 0.7 | 0.7 | 1.0 | 1.8 | 0.8 | 4.4 | 1.0 | 0.7 | 1.0 | 1.5 | 0.8 | 1.2 | 1.0 |
| IL-13 | 1.0 | 1.0 | 1.1 | 0.9 | 0.6 | 0.9 | 1.0 | 0.9 | 1.0 | 1.0 | 0.7 | 1.0 | 2.5 | 1.0 | 1.0 | 0.4 |
| IL-15 | 1.0 | 1.0 | 0.8 | 1.0 | 0.9 | 0.9 | 1.0 | 0.9 | 1.0 | 1.0 | 0.7 | 1.0 | 1.0 | 0.8 | 0.9 | 0.1 |
| sCD40L | 1.1 | 1.4 | 1.1 | 1.0 | 0.8 | 1.0 | 1.8 | 0.4 | 0.2 | 1.0 | 1.0 | 0.2 | 0.7 | 1.0 | 0.9 | 0.4 |
| IL-17A | 1.0 | 1.0 | 1.0 | 0.9 | 0.7 | 0.8 | 1.1 | 0.8 | 4.2 | 1.0 | 0.8 | 1.0 | 1.3 | 0.8 | 1.2 | 0.9 |
| IL-1RA | 1.8 | 1.4 | 1.1 | 0.8 | 0.8 | 0.9 | 1.7 | 0.7 | 1.4 | 1.2 | 0.5 | 1.0 | 1.0 | 0.8 | 1.1 | 0.4 |
| IL-1a | 0.8 | 1.0 | 0.9 | 0.9 | 0.9 | 0.9 | 1.2 | 0.9 | 1.1 | 0.6 | 0.5 | 0.6 | 1.0 | 0.8 | 0.9 | 0.2 |
| IL-9 | 1.0 | 1.0 | 1.0 | 1.0 | 0.9 | 0.9 | 1.0 | 0.9 | 1.1 | 1.0 | 0.5 | 1.0 | 1.0 | 0.7 | 0.9 | 0.1 |
| IL-1b | 1.0 | 1.0 | 0.9 | 1.0 | 0.8 | 0.9 | 1.0 | 0.9 | 0.9 | 1.0 | 0.7 | 1.0 | 1.0 | 0.7 | 0.9 | 0.1 |
| IL-2 | 1.0 | 1.0 | 0.9 | 1.0 | 0.9 | 0.9 | 1.0 | 0.9 | 1.2 | 1.0 | 0.6 | 1.0 | 1.0 | 0.8 | 0.9 | 0.1 |
| IL-3 | 1.0 | 1.0 | 1.0 | 1.0 | 1.0 | 0.9 | 1.0 | 0.7 | 1.0 | 1.0 | 1.0 | 1.0 | 1.0 | 0.9 | 1.0 | 0.1 |
| IL-4 | 1.0 | 1.0 | 0.9 | 0.8 | 0.8 | 1.0 | 2.8 | 0.8 | 1.1 | 1.0 | 0.8 | 1.0 | 1.0 | 1.0 | 1.1 | 0.6 |
| IL-5 | 1.0 | 1.0 | 1.0 | 1.0 | 0.8 | 0.9 | 1.0 | 0.5 | 1.9 | 1.0 | 0.4 | 1.0 | 1.0 | 0.9 | 1.0 | 0.3 |
| IL-6 | 1.9 | 1.0 | 0.9 | 1.0 | 0.7 | 1.0 | 1.9 | 0.9 | 1.8 | 1.0 | 0.6 | 1.0 | 1.0 | 1.0 | 1.1 | 0.4 |
| IL-7 | 1.0 | 1.0 | 1.0 | 1.0 | 0.9 | 0.9 | 1.4 | 0.7 | 1.8 | 1.0 | 0.7 | 1.0 | 1.0 | 0.7 | 1.0 | 0.3 |
| IL-8 | 1.0 | 1.0 | 1.4 | 1.1 | 0.8 | 0.9 | 1.0 | 0.9 | 2.1 | 1.1 | 0.8 | 1.1 | 1.0 | 0.8 | 1.1 | 0.3 |
| IP-10 | 0.6 | 0.7 | 0.8 | 0.9 | 1.0 | 0.8 | 0.8 | 0.8 | 1.0 | 0.6 | 0.6 | 0.7 | 0.9 | 0.9 | 0.8 | 0.1 |
| MCP-1 | 1.1 | 0.9 | 0.9 | 0.9 | 0.9 | 0.8 | 0.9 | 0.8 | 0.8 | 1.0 | 1.1 | 0.9 | 1.2 | 0.7 | 0.9 | 0.1 |
| MIP-1a | 1.0 | 1.0 | 1.0 | 1.0 | 0.7 | 1.0 | 1.0 | 0.9 | 1.7 | 1.0 | 0.8 | 1.0 | 1.0 | 0.9 | 1.0 | 0.2 |
| MIP-1b | 1.0 | 1.0 | 1.0 | 0.7 | 0.5 | 0.9 | 1.4 | 0.8 | 2.1 | 1.0 | 0.7 | 1.0 | 1.2 | 0.9 | 1.0 | 0.4 |
| TNFa | 1.0 | 1.0 | 0.9 | 0.9 | 0.9 | 0.9 | 1.1 | 0.9 | 2.1 | 0.9 | 0.6 | 0.9 | 1.0 | 1.1 | 1.0 | 0.3 |
| TNFb | 1.0 | 1.0 | 1.1 | 1.0 | 0.9 | 0.9 | 1.5 | 1.0 | 1.2 | 1.0 | 0.5 | 1.0 | 1.0 | 0.9 | 1.0 | 0.2 |
| VEGF | 3.4 | 0.8 | 1.2 | 0.9 | 0.7 | 0.9 | 1.2 | 1.0 | 2.0 | 1.2 | 0.8 | 1.2 | 1.2 | 1.0 | 1.3 | 0.7 |

The second-to-last column is Mean 2 hr Post dose placebo.
The last column is SD 2 hr Post dose placebo.

TABLE 4
Plasma cytokine and chemokine fold-change at 4 h post dose peptide composition or
placebo compared to pre-dose

| | Cohort | | | | | | | | | |
|---|---|---|---|---|---|---|---|---|---|---|
| | 1 | 1 | 1 | 1 | 1 | 1 | 1 | 1 | | |
| | Dose | | | | | | | | | |
| | 150 | 150 | 150 | 150 | 150 | 150 | 150 | 150 | | |
| EGF | 1.2 | 1.5 | 4.2 | 2.9 | 1.7 | 2.2 | 1.2 | 1.7 | 2.1 | 1.0 |
| FGF-2 | 1.1 | 2.7 | 1.6 | 1.2 | 15.8 | 1.3 | 1.3 | 1.3 | 3.3 | 5.1 |
| EOTAXIN | 1.5 | 4.1 | 3.6 | 1.5 | 14.6 | 2.6 | 2.2 | 2.1 | 4.0 | 4.4 |
| TGF-a | 1.0 | 1.9 | 1.0 | 26.3 | 2.1 | 1.1 | 1.0 | 1.0 | 4.4 | 8.8 |
| G-CSF | 1.7 | 7.8 | 3.5 | 1.2 | 81.6 | 2.9 | 2.4 | 2.4 | 12.9 | 27.8 |
| Flt-3L | 1.0 | 9.8 | 3.4 | 1.2 | 5.4 | 0.8 | 1.7 | 1.0 | 3.0 | 3.2 |
| GM-CSF | 2.9 | 4.7 | 10.1 | 1.5 | 5.1 | 3.9 | 5.4 | 1.8 | 4.4 | 2.7 |
| FRACTALKINE | 1.4 | 2.3 | 2.4 | 1.3 | 3.2 | 1.9 | 2.5 | 2.9 | 2.2 | 0.7 |
| IFNa2 | 1.6 | 2.4 | 3.9 | 1.1 | 7.0 | 2.7 | 3.4 | 2.2 | 3.1 | 1.8 |
| IFNg | 1.3 | 2.0 | 2.0 | 3.3 | 9.2 | 1.5 | 1.6 | 1.6 | 2.8 | 2.6 |
| GRO | 1.1 | 3.1 | 3.4 | 1.6 | 4.8 | 2.3 | 1.2 | 2.0 | 2.5 | 1.3 |
| IL-10 | 1.9 | 23.9 | 6.9 | 1.1 | 30.3 | 7.3 | 5.5 | 16.6 | 11.7 | 10.7 |
| MCP-3 | 1.1 | 1.2 | 2.3 | 1.6 | 2.8 | 1.7 | 1.8 | 1.8 | 1.8 | 0.6 |
| IL-12(P40) | 1.2 | 2.0 | 1.6 | 1.2 | 1.0 | 1.7 | 2.8 | 1.3 | 1.6 | 0.6 |
| MDC | 1.0 | 0.9 | 0.8 | 2.2 | 1.2 | 1.0 | 1.1 | 1.1 | 1.2 | 0.4 |
| IL-12P70 | 1.9 | 4.4 | 4.3 | 1.2 | 1.0 | 2.8 | 2.0 | 1.8 | 2.4 | 1.3 |
| IL-13 | 1.2 | 1.4 | 1.2 | 1.1 | 1.0 | 2.4 | 1.0 | 1.9 | 1.4 | 0.5 |
| IL-15 | 1.0 | 2.3 | 2.1 | 1.2 | 1.0 | 1.8 | 1.0 | 1.5 | 1.5 | 0.5 |
| sCD40L | 1.7 | 1.4 | 4.4 | 0.6 | 2.3 | 1.0 | 1.0 | 1.8 | 1.8 | 1.2 |
| IL-17A | 1.0 | 8.4 | 1.8 | 4.2 | 2.0 | 1.1 | 2.2 | 1.2 | 2.7 | 2.5 |
| IL-1RA | 1.4 | 4.4 | 3.9 | 1.3 | 13.1 | 2.8 | 2.6 | 2.2 | 4.0 | 3.9 |
| IL-1a | 1.7 | 3.0 | 3.5 | 1.3 | 9.2 | 2.9 | 6.7 | 3.2 | 4.0 | 2.7 |
| IL-9 | 1.0 | 2.2 | 1.0 | 1.2 | 1.0 | 1.4 | 1.0 | 1.0 | 1.2 | 0.4 |
| IL-1b | 1.0 | 2.7 | 2.5 | 1.2 | 1.0 | 1.4 | 1.0 | 1.4 | 1.5 | 0.7 |
| IL-2 | 9.4 | 38.4 | 49.7 | 3.2 | 56.2 | 50.7 | 13.2 | 6.8 | 28.5 | 22.4 |
| IL-3 | 1.0 | 2.5 | 1.0 | 1.2 | 1.0 | 1.0 | 1.0 | 1.0 | 1.2 | 0.5 |
| IL-4 | 2.7 | 1.6 | 3.4 | 1.1 | 1.0 | 1.0 | 1.0 | 2.3 | 1.8 | 0.9 |
| IL-5 | 1.0 | 1.7 | 1.0 | 1.2 | 1.0 | 1.2 | 1.0 | 1.0 | 1.1 | 0.3 |
| IL-6 | 2.0 | 9.9 | 8.7 | 1.5 | 67.8 | 3.8 | 3.7 | 9.1 | 13.3 | 22.3 |
| IL-7 | 1.0 | 4.5 | 3.4 | 1.2 | 3.9 | 2.7 | 1.1 | 2.4 | 2.5 | 1.4 |
| IL-8 | 8.6 | 49.7 | 36.7 | 3.2 | 239.6 | 3.7 | 40.9 | 19.6 | 50.2 | 78.5 |
| IP-10 | 1.6 | 9.3 | 8.6 | 1.7 | 8.8 | 3.7 | 3.0 | 7.5 | 5.5 | 3.3 |
| MCP-1 | 3.0 | 30.4 | 33.7 | 5.9 | 26.4 | 12.5 | 8.3 | 9.2 | 16.2 | 12.0 |
| MIP-1a | 2.4 | 3.3 | 2.1 | 1.6 | 16.7 | 3.4 | 1.8 | 1.4 | 4.1 | 5.2 |
| MIP-1b | 2.9 | 4.4 | 5.6 | 1.8 | 14.8 | 3.2 | 4.3 | 3.3 | 5.0 | 4.1 |
| TNFa | 2.9 | 7.4 | 7.3 | 1.8 | 7.2 | 3.6 | 4.0 | 3.5 | 4.7 | 2.2 |
| TNFb | 1.1 | 1.4 | 2.8 | 1.4 | 1.0 | 2.9 | 1.0 | 2.1 | 1.7 | 0.8 |
| VEGF | 1.3 | 7.7 | 1.5 | 1.7 | 27.5 | 1.8 | 1.9 | 1.4 | 5.6 | 9.1 |

The second-to-last column is Mean 4 hr Post dose:Cohort 1.
The last column is SD 4 hr Post dose:Cohort 1.

| | Cohort | | | | | | | | | | | |
|---|---|---|---|---|---|---|---|---|---|---|---|---|
| | 2 | 2 | 2 | 2 | 2 | 2 | 2 | 2 | 2 | 2 | | |
| | Dose | | | | | | | | | | | |
| | 300 | 300 | 300 | 300 | 300 | 300 | 300 | 300 | 300 | 300 | | |
| EGF | 0.3 | 0.9 | 1.0 | 0.8 | 1.3 | 1.8 | 1.2 | 0.7 | ND | ND | 1.0 | 0.5 |
| FGF-2 | 0.8 | 1.1 | 1.6 | 0.9 | 1.2 | 1.2 | 1.2 | 0.6 | ND | ND | 1.1 | 0.4 |
| EOTAXIN | 1.4 | 2.1 | 2.9 | 1.2 | 3.0 | 1.3 | 1.7 | 1.7 | ND | ND | 1.9 | 0.8 |
| TGF-a | 1.0 | 1.8 | 2.4 | 1.0 | 1.3 | 1.7 | 1.4 | 0.4 | ND | ND | 1.4 | 0.7 |
| G-CSF | 1.2 | 3.1 | 2.1 | 0.8 | 1.3 | 1.3 | 1.1 | 0.8 | ND | ND | 1.5 | 0.5 |
| Flt-3L | 0.8 | 4.1 | 6.9 | 1.0 | 1.0 | 1.4 | 1.0 | 0.3 | ND | ND | 2.1 | 2.4 |
| GM-CSF | 1.0 | 2.9 | 2.0 | 1.1 | 2.0 | 1.3 | 1.4 | 0.4 | ND | ND | 1.5 | 0.6 |
| FRACTALKINE | 0.9 | 2.0 | 1.7 | 1.1 | 1.2 | 1.2 | 1.1 | 0.5 | ND | ND | 1.2 | 0.4 |
| IFNa2 | 0.9 | 2.2 | 1.5 | 0.9 | 1.2 | 1.3 | 1.3 | 0.6 | ND | ND | 1.2 | 0.3 |
| IFNg | 1.0 | 2.0 | 1.6 | 1.0 | 1.2 | 1.4 | 1.4 | 0.6 | ND | ND | 1.3 | 0.4 |
| GRO | 0.3 | 1.0 | 1.1 | 1.0 | 5.1 | 1.4 | 1.3 | 2.7 | ND | ND | 1.7 | 1.6 |
| IL-10 | 0.9 | 3.4 | 4.4 | 1.0 | 2.2 | 0.9 | 1.2 | 0.4 | ND | ND | 1.8 | 1.5 |
| MCP-3 | 0.9 | 1.2 | 1.1 | 0.7 | 1.0 | 1.2 | 1.4 | 0.8 | ND | ND | 1.0 | 0.2 |
| IL-12(P40) | 0.9 | 1.2 | 1.5 | 0.5 | 1.0 | 1.3 | 1.1 | 0.9 | ND | ND | 1.1 | 0.3 |
| MDC | 0.6 | 1.0 | 1.2 | 0.9 | 0.9 | 0.8 | 1.1 | 0.9 | ND | ND | 0.9 | 0.1 |
| IL-12P70 | 1.0 | 2.5 | 2.5 | 1.0 | 1.0 | 1.4 | 1.3 | 0.3 | ND | ND | 1.4 | 0.7 |
| IL-13 | 0.9 | 1.2 | 1.1 | 1.0 | 0.9 | 1.1 | 1.4 | 0.3 | ND | ND | 1.0 | 0.4 |
| IL-15 | 0.8 | 1.2 | 2.8 | 1.0 | 1.0 | 1.0 | 1.2 | 0.3 | ND | ND | 1.2 | 0.8 |
| sCD40L | 0.4 | 1.1 | 0.7 | 1.0 | 1.9 | 2.3 | 1.4 | 0.8 | ND | ND | 1.2 | 0.6 |
| IL-17A | 1.0 | 3.1 | 1.6 | 1.0 | 1.3 | 1.3 | 1.3 | 0.6 | ND | ND | 1.4 | 0.3 |
| IL-1RA | 0.9 | 2.1 | 2.2 | 0.8 | 1.4 | 1.4 | 1.3 | 0.6 | ND | ND | 1.4 | 0.6 |
| IL-1a | 1.0 | 1.7 | 2.6 | 1.0 | 1.1 | 1.0 | 1.1 | 0.7 | ND | ND | 1.3 | 0.7 |
| IL-9 | 1.0 | 1.4 | 1.8 | 1.0 | 1.1 | 1.0 | 1.2 | 0.4 | ND | ND | 1.1 | 0.5 |
| IL-1b | 1.0 | 1.2 | 2.1 | 1.0 | 1.0 | 1.0 | 1.1 | 0.4 | ND | ND | 1.1 | 0.5 |
| IL-2 | 1.0 | 22.2 | 34.0 | 1.1 | 6.7 | 1.0 | 1.6 | 0.5 | ND | ND | 8.5 | 13.2 |
| IL-3 | 1.0 | 1.7 | 2.0 | 1.0 | 1.0 | 0.9 | 1.3 | 1.0 | ND | ND | 1.2 | 0.4 |
| IL-4 | 0.9 | 1.5 | 1.4 | 1.0 | 1.0 | 1.2 | 1.4 | 0.7 | ND | ND | 1.1 | 0.3 |
| IL-5 | 1.0 | 1.1 | 1.6 | 1.0 | 1.1 | 1.4 | 1.6 | 0.4 | ND | ND | 1.1 | 0.5 |
| IL-6 | 9.9 | 2.5 | 8.0 | 1.0 | 2.2 | 1.0 | 1.3 | 0.6 | ND | ND | 3.3 | 2.8 |
| IL-7 | 1.0 | 3.1 | 2.4 | 1.0 | 1.6 | 1.9 | 1.4 | 0.7 | ND | ND | 1.6 | 0.6 |
| IL-8 | 2.0 | 26.2 | 4.9 | 7.6 | 18.9 | 1.3 | 3.1 | 1.4 | ND | ND | 8.2 | 6.7 |
| IP-10 | 1.0 | 6.9 | 12.3 | 1.9 | 2.2 | 0.4 | 1.2 | 0.8 | ND | ND | 3.4 | 4.5 |
| MCP-1 | 2.1 | 11.8 | 17.1 | 2.2 | 17.3 | 1.3 | 2.8 | 5.5 | ND | ND | 7.5 | 7.5 |
| MIP-1a | 1.0 | 2.4 | 2.2 | 1.0 | 1.8 | 1.1 | 1.4 | 0.8 | ND | ND | 1.5 | 0.5 |
| MIP-1b | 0.9 | 4.2 | 2.1 | 2.0 | 3.2 | 1.5 | 1.3 | 0.7 | ND | ND | 2.0 | 0.9 |
| TNFa | 0.9 | 5.8 | 3.7 | 1.5 | 2.9 | 1.3 | 1.6 | 0.5 | ND | ND | 2.3 | 1.2 |
| TNFb | 0.9 | 1.2 | 1.2 | 1.0 | 1.0 | 1.4 | 1.5 | 0.7 | ND | ND | 1.1 | 0.3 |
| VEGF | 1.0 | 1.4 | 1.6 | 1.0 | 1.2 | 1.4 | 1.1 | 0.6 | ND | ND | 1.2 | 0.3 |

The second-to-last column is Mean 4 hr Post dose:Cohort 2.
The last column is SD 4 hr Post dose:Cohort 2.

| | Cohort | | | | | | | | |
|---|---|---|---|---|---|---|---|---|---|
| | 7 | 7 | 7 | 7 | 7 | 7 | 7 | | |
| | Dose | | | | | | | | |
| | 150 | 150 | 150 | 150 | 150 | 150 | 150 | | |
| EGF | 0.6 | 0.8 | 3.0 | 3.8 | 0.9 | 0.5 | 0.8 | 1.4 | 1.3 |
| FGF-2 | 0.6 | 1.4 | 1.1 | 1.6 | 1.0 | 1.0 | 1.1 | 1.0 | 0.4 |
| EOTAXIN | 1.0 | 1.3 | 2.0 | 1.5 | 1.5 | 2.4 | 1.4 | 1.5 | 0.5 |
| TGF-a | 1.0 | 1.0 | 1.0 | 1.0 | 1.1 | 1.0 | 1.1 | 1.0 | 0.1 |
| G-CSF | 0.8 | 1.7 | 1.2 | 0.9 | 1.0 | 1.5 | 1.1 | 1.1 | 0.4 |
| Flt-3L | 1.0 | 1.0 | 3.9 | 0.7 | 0.6 | 2.3 | 1.2 | 1.6 | 1.1 |
| GM-CSF | 0.9 | 1.5 | 2.0 | 1.1 | 1.0 | 1.5 | 1.3 | 1.2 | 0.4 |
| FRACTALKINE | 1.0 | 1.3 | 1.2 | 0.9 | 0.9 | 1.6 | 1.2 | 1.0 | 0.4 |
| IFNa2 | 0.8 | 1.5 | 1.5 | 1.3 | 0.9 | 1.3 | 1.3 | 1.1 | 0.4 |
| IFNg | 0.7 | 1.4 | 1.0 | 1.1 | 1.0 | 1.5 | 1.0 | 1.0 | 0.4 |
| GRO | 0.8 | 0.9 | 1.4 | 2.0 | 1.0 | 0.7 | 0.6 | 1.1 | 0.5 |
| IL-10 | 0.9 | 1.0 | 1.7 | 1.0 | 1.0 | 1.0 | 1.1 | 1.2 | 0.3 |
| MCP-3 | 0.7 | 1.5 | 1.4 | 1.2 | 1.0 | 1.3 | 1.2 | 1.1 | 0.4 |
| IL-12(P40) | 0.8 | 1.3 | 1.1 | 0.9 | 1.0 | 1.1 | 1.1 | 1.0 | 0.3 |
| MDC | 1.0 | 1.1 | 1.1 | 0.9 | 1.0 | 1.4 | 1.0 | 1.0 | 0.4 |
| IL-12P70 | 1.0 | 1.5 | 1.6 | 1.0 | 0.9 | 1.9 | 1.2 | 1.2 | 0.4 |
| IL-13 | 1.0 | 1.0 | 1.4 | 0.8 | 0.9 | 1.0 | 1.2 | 1.0 | 0.3 |
| IL-15 | 1.0 | 1.0 | 1.3 | 1.0 | 1.0 | 1.0 | 1.1 | 1.0 | 0.1 |
| sCD40L | 0.8 | 1.1 | 2.6 | 3.5 | 0.7 | 0.4 | 0.6 | 1.3 | 1.1 |
| IL-17A | 1.0 | 1.0 | 1.0 | 1.0 | 0.9 | 1.7 | 1.1 | 1.0 | 0.4 |
| IL-1RA | 0.9 | 1.6 | 1.5 | 1.3 | 1.0 | 1.5 | 1.2 | 1.2 | 0.4 |
| IL-1a | 1.0 | 2.0 | 1.3 | 1.0 | 0.9 | 1.2 | 1.1 | 1.1 | 0.4 |
| IL-9 | 1.0 | 1.0 | 1.0 | 1.0 | 1.0 | 1.0 | 1.1 | 0.9 | 0.2 |
| IL-1b | 1.0 | 1.0 | 1.4 | 1.0 | 1.0 | 1.0 | 1.1 | 1.0 | 0.2 |
| IL-2 | 1.0 | 11.1 | 12.9 | 1.0 | 1.3 | 3.5 | 2.0 | 5.7 | 5.6 |
| IL-3 | 1.0 | 1.0 | 1.0 | 1.0 | 0.9 | 1.0 | 1.1 | 0.9 | 0.2 |
| IL-4 | 1.0 | 1.1 | 1.2 | 0.8 | 0.9 | 1.0 | 1.1 | 0.9 | 0.3 |
| IL-5 | 1.0 | 1.0 | 1.0 | 1.0 | 1.0 | 1.0 | 1.3 | 1.0 | 0.2 |
| IL-6 | 1.0 | 1.1 | 5.8 | 1.4 | 1.3 | 1.0 | 1.3 | 2.0 | 1.7 |
| IL-7 | 1.0 | 1.0 | 1.0 | 1.0 | 0.8 | 1.0 | 1.2 | 1.0 | 0.2 |
| IL-8 | 1.6 | 14.2 | 11.4 | 1.0 | 2.9 | 3.9 | 2.4 | 5.5 | 4.9 |
| IP-10 | 0.7 | 1.9 | 1.5 | 0.8 | 0.9 | 1.4 | 1.2 | 1.6 | 1.2 |
| MCP-1 | 1.1 | 4.7 | 6.8 | 1.3 | 2.1 | 4.5 | 3.6 | 3.9 | 2.4 |
| MIP-1a | 1.0 | 1.2 | 2.1 | 1.0 | 1.0 | 1.7 | 1.2 | 1.2 | 0.5 |
| MIP-1b | 1.0 | 2.5 | 3.6 | 1.0 | 1.3 | 1.9 | 1.2 | 1.7 | 1.0 |
| TNFa | 1.0 | 2.5 | 2.5 | 1.0 | 1.2 | 2.1 | 1.4 | 1.6 | 0.6 |
| TNFb | 1.0 | 1.0 | 1.5 | 1.1 | 1.0 | 1.0 | 1.1 | 1.0 | 0.3 |
| VEGF | 0.6 | 1.4 | 1.1 | 1.1 | 1.0 | 1.2 | 1.0 | 1.0 | 0.4 |

The second-to-last column is Mean 4 hr Post dose:Cohort 7.
The last column is SD 4 hr Post dose:Cohort 7.

| | Cohort | | | | | | | | | | | | | | | |
|---|---|---|---|---|---|---|---|---|---|---|---|---|---|---|---|---|
| | 1 | 1 | 1 | 1 | 2 | 2 | 2 | 7 | 7 | 7 | 7 | 7 | 7 | 7 | | |
| | Dose Nexvax2/ug | | | | | | | | | | | | | | | |
| | 0 | 0 | 0 | 0 | 0 | 0 | 0 | 0 | 0 | 0 | 0 | 0 | 0 | 0 | | |
| EGF | 1.1 | 1.4 | 2.6 | 0.7 | 0.7 | 0.9 | 1.0 | 0.9 | 0.5 | 1.8 | 0.9 | 0.2 | 0.5 | 0.7 | 1.0 | 0.6 |
| FGF-2 | 1.2 | 1.0 | 1.1 | 0.8 | 0.8 | 0.9 | 1.4 | 0.8 | 1.7 | 1.2 | NR | 0.9 | 1.0 | 0.9 | 1.1 | 0.3 |
| EOTAXIN | 1.2 | 1.0 | 1.0 | 1.0 | 1.0 | 0.9 | 1.0 | 0.9 | 0.9 | 0.9 | 0.7 | 1.1 | 1.0 | 0.7 | 1.0 | 0.1 |
| TGF-a | 1.0 | 1.0 | 1.0 | 1.0 | 1.0 | 1.0 | 1.0 | 0.7 | 1.0 | 1.0 | 0.5 | 1.0 | 1.0 | 0.8 | 0.9 | 0.2 |
| G-CSF | 1.4 | 1.2 | 1.1 | 0.9 | 0.9 | 0.9 | 1.1 | 0.9 | 1.0 | 1.1 | 0.6 | 0.8 | 0.8 | 0.8 | 1.0 | 0.2 |
| Flt-3L | 1.0 | 1.0 | 1.0 | 0.9 | 1.0 | 0.8 | 0.9 | 0.7 | 7.1 | 1.0 | 0.4 | 1.0 | 1.0 | 0.7 | 1.3 | 1.7 |
| GM-CSF | 1.4 | 1.1 | 1.1 | 0.9 | 0.7 | 0.9 | 1.4 | 0.9 | 1.5 | 1.1 | NR | 1.0 | 1.0 | 0.7 | 1.1 | 0.3 |
| FRACTALKINE | 1.3 | 0.8 | 1.0 | 0.8 | 0.9 | 0.9 | 1.3 | 0.8 | 1.4 | 1.0 | 0.6 | 0.8 | 1.3 | 0.8 | 1.0 | 0.3 |
| IFNa2 | 1.5 | 1.1 | 1.2 | 0.8 | 0.8 | 0.9 | 1.8 | 0.7 | 1.5 | 1.0 | 0.7 | 1.0 | 1.3 | 0.8 | 1.1 | 0.3 |
| IFNg | 0.9 | 0.9 | 1.3 | 0.9 | 0.6 | 0.9 | 1.1 | 0.9 | 3.0 | 1.0 | 1.0 | 1.2 | 2.4 | 0.8 | 1.2 | 0.7 |
| GRO | 1.6 | 1.2 | 1.4 | 0.9 | 0.9 | 1.1 | 1.2 | 0.7 | 0.3 | 1.2 | 1.4 | 1.1 | 0.5 | 0.6 | 1.0 | 0.4 |
| IL-10 | 1.0 | 1.0 | 0.9 | 0.8 | 1.0 | 1.0 | 1.8 | 0.8 | 1.0 | 1.0 | 0.8 | 1.0 | 1.0 | 0.7 | 1.0 | 0.3 |
| MCP-3 | 1.8 | 1.2 | 1.2 | 1.0 | 0.9 | 1.0 | 1.6 | 0.9 | 1.3 | 1.0 | 0.7 | 1.0 | 1.0 | 0.9 | 1.1 | 0.3 |
| IL-12(P40) | 1.1 | 0.8 | 1.0 | 0.7 | 0.9 | 1.0 | 2.1 | 1.0 | 1.0 | 1.0 | 0.6 | 1.0 | 1.0 | 0.9 | 1.0 | 0.3 |
| MDC | 0.9 | 0.9 | 1.1 | 0.8 | 1.1 | 0.9 | 1.1 | 0.9 | 1.2 | 0.9 | 0.8 | 0.9 | 1.0 | 0.8 | 1.0 | 0.1 |
| IL-12P70 | 1.0 | 1.0 | 1.0 | 0.7 | 0.7 | 1.0 | 1.8 | 0.8 | 4.4 | 1.0 | 0.7 | 1.0 | 1.5 | 0.8 | 1.2 | 1.0 |
| IL-13 | 1.0 | 1.0 | 1.1 | 0.9 | 0.6 | 0.9 | 1.0 | 0.9 | 1.0 | 1.0 | 0.7 | 1.0 | 2.5 | 1.0 | 1.0 | 0.4 |
| IL-15 | 1.0 | 1.0 | 0.8 | 1.0 | 0.9 | 0.9 | 1.0 | 0.9 | 1.0 | 1.0 | 0.7 | 1.0 | 1.0 | 0.8 | 0.9 | 0.1 |
| sCD40L | 1.1 | 1.4 | 1.1 | 1.0 | 0.8 | 1.0 | 1.8 | 0.4 | 0.2 | 1.0 | 1.0 | 0.2 | 0.7 | 1.0 | 0.9 | 0.4 |
| IL-17A | 1.0 | 1.0 | 1.0 | 0.9 | 0.7 | 0.8 | 1.1 | 0.8 | 4.2 | 1.0 | 0.8 | 1.0 | 1.3 | 0.8 | 1.2 | 0.9 |
| IL-1RA | 1.8 | 1.4 | 1.1 | 0.8 | 0.8 | 0.9 | 1.7 | 0.7 | 1.4 | 1.2 | 0.5 | 1.0 | 1.0 | 0.8 | 1.1 | 0.4 |
| IL-1a | 0.8 | 1.0 | 0.9 | 0.9 | 0.9 | 0.9 | 1.2 | 0.9 | 1.1 | 0.6 | 0.5 | 0.6 | 1.0 | 0.8 | 0.9 | 0.2 |
| IL-9 | 1.0 | 1.0 | 1.0 | 1.0 | 0.9 | 0.9 | 1.0 | 0.9 | 1.1 | 1.0 | 0.5 | 1.0 | 1.0 | 0.7 | 0.9 | 0.1 |
| IL-1b | 1.0 | 1.0 | 0.9 | 1.0 | 0.8 | 0.9 | 1.0 | 0.9 | 0.9 | 1.0 | 0.7 | 1.0 | 1.0 | 0.7 | 0.9 | 0.1 |
| IL-2 | 1.0 | 1.0 | 0.9 | 1.0 | 0.9 | 0.9 | 1.0 | 0.9 | 1.2 | 1.0 | 0.6 | 1.0 | 1.0 | 0.8 | 0.9 | 0.1 |
| IL-3 | 1.0 | 1.0 | 1.0 | 1.0 | 1.0 | 0.9 | 1.0 | 0.7 | 1.0 | 1.0 | 1.0 | 1.0 | 1.0 | 0.9 | 1.0 | 0.1 |
| IL-4 | 1.0 | 1.0 | 0.9 | 0.8 | 0.8 | 1.0 | 2.8 | 0.8 | 2.1 | 1.0 | 0.8 | 1.0 | 1.0 | 1.0 | 1.1 | 0.6 |
| IL-5 | 1.0 | 1.0 | 1.0 | 1.0 | 0.8 | 0.9 | 1.0 | 0.6 | 1.9 | 1.0 | 0.4 | 1.0 | 1.0 | 0.9 | 1.0 | 0.3 |
| IL-6 | 1.9 | 1.0 | 0.9 | 1.0 | 0.7 | 1.0 | 1.9 | 0.9 | 1.8 | 1.0 | 0.6 | 1.0 | 1.0 | 1.0 | 1.1 | 0.4 |
| IL-7 | 1.0 | 1.0 | 1.0 | 1.0 | 0.9 | 0.9 | 1.4 | 0.7 | 1.8 | 1.0 | 0.7 | 1.0 | 1.0 | 0.7 | 1.0 | 0.3 |
| IL-8 | 1.0 | 1.0 | 1.4 | 1.1 | 0.8 | 0.9 | 1.0 | 0.9 | 2.1 | 1.1 | 0.8 | 1.1 | 1.0 | 0.8 | 1.1 | 0.3 |
| IP-10 | 0.6 | 0.7 | 0.8 | 0.9 | 1.0 | 0.8 | 0.8 | 0.8 | 1.0 | 0.6 | 0.6 | 0.7 | 0.9 | 0.9 | 0.8 | 0.1 |
| MCP-1 | 1.1 | 0.9 | 0.9 | 0.9 | 0.9 | 0.8 | 0.9 | 0.8 | 0.8 | 1.0 | 1.1 | 0.9 | 1.2 | 0.7 | 0.9 | 0.1 |
| MIP-1a | 1.0 | 1.0 | 1.0 | 1.0 | 0.7 | 1.0 | 1.0 | 0.9 | 1.7 | 1.0 | 0.8 | 1.0 | 1.0 | 0.9 | 1.0 | 0.2 |
| MIP-1b | 1.0 | 1.0 | 1.0 | 0.7 | 0.5 | 0.9 | 1.4 | 0.8 | 2.1 | 1.0 | 0.7 | 1.0 | 1.2 | 0.9 | 1.0 | 0.4 |
| TNFa | 1.0 | 1.0 | 0.9 | 0.9 | 0.9 | 0.9 | 1.1 | 0.9 | 2.1 | 0.9 | 0.6 | 0.9 | 1.0 | 1.1 | 1.0 | 0.3 |
| TNFb | 1.0 | 1.0 | 1.1 | 1.0 | 0.9 | 0.9 | 1.5 | 1.0 | 1.2 | 1.0 | 0.5 | 1.0 | 1.0 | 0.9 | 1.0 | 0.2 |
| VEGF | 3.4 | 0.8 | 1.2 | 0.9 | 0.7 | 0.9 | 1.2 | 1.0 | 2.0 | 1.2 | 0.8 | 1.2 | 1.2 | 1.0 | 1.3 | 0.7 |

The second-to-last column is Mean 4 hr Post dose placebo.
The last column is SD 4 hr Post dose placebo.

TABLE 5
Plasma cytokine and chemokine fold-change at 6 h post dose peptide composition or
placebo compared to pre-dose

| | Cohort | | | | | | | | | |
|---|---|---|---|---|---|---|---|---|---|---|
| | 1 | 1 | 1 | 1 | 1 | 1 | 1 | 1 | | |
| | Dose | | | | | | | | | |
| | 150 | 150 | 150 | 150 | 150 | 150 | 150 | 150 | | |
| EGF | 1.0 | 2.5 | 1.4 | 1.8 | 2.0 | 1.7 | 2.2 | 0.3 | 1.6 | 0.7 |
| FGF-2 | 1.4 | 2.4 | 1.6 | 1.1 | 4.0 | 1.2 | 1.3 | 1.2 | 1.8 | 1.0 |
| EOTAXIN | 4.6 | 3.9 | 4.1 | 1.6 | 5.2 | 2.0 | 1.6 | 3.5 | 3.3 | 1.4 |
| TGF-a | 1.0 | 2.9 | 1.0 | 6.5 | 3.8 | 1.0 | 1.0 | 1.4 | 2.3 | 2.0 |
| G-CSF | 5.4 | 20.2 | 13.9 | 1.3 | 95.1 | 3.2 | 2.6 | 2.7 | 18.0 | 31.8 |
| Flt-3L | 1.0 | 6.7 | 3.6 | 1.3 | 5.9 | 1.0 | 1.0 | 1.0 | 2.7 | 2.4 |
| GM-CSF | 4.6 | 3.1 | 6.5 | 1.3 | 4.5 | 2.6 | 3.3 | 1.8 | 3.5 | 1.7 |
| FRACTALKINE | 1.6 | 1.9 | 2.3 | 1.3 | 5.2 | 1.5 | 2.2 | 2.9 | 2.4 | 1.3 |
| IFNa2 | 2.8 | 1.9 | 3.9 | 1.1 | 9.3 | 2.1 | 2.6 | 2.3 | 3.3 | 2.6 |
| IFNg | 2.9 | 1.6 | 1.7 | 1.9 | 7.7 | 1.4 | 1.2 | 2.5 | 2.6 | 2.2 |
| GRO | 3.4 | 2.7 | 3.1 | 1.5 | 2.0 | 1.7 | 1.5 | 1.3 | 2.2 | 0.8 |
| IL-10 | 8.4 | 6.1 | 7.8 | 1.4 | 8.9 | 5.3 | 4.3 | 7.4 | 6.2 | 2.5 |
| MCP-3 | 1.1 | 1.1 | 2.3 | 1.4 | 8.2 | 1.5 | 1.5 | 2.0 | 2.4 | 2.4 |
| IL-12(P40) | 1.8 | 1.6 | 1.5 | 1.2 | 3.8 | 1.6 | 2.2 | 1.5 | 1.9 | 0.8 |
| MDC | 1.2 | 1.2 | 1.1 | 1.6 | 1.3 | 1.1 | 1.1 | 1.3 | 1.2 | 0.2 |
| IL-12P70 | 2.7 | 3.2 | 4.5 | 1.3 | 4.0 | 2.4 | 1.6 | 2.1 | 2.7 | 1.1 |
| IL-13 | 1.4 | 1.1 | 0.7 | 1.1 | 1.0 | 2.0 | 1.0 | 2.1 | 1.3 | 0.5 |
| IL-15 | 1.1 | 1.9 | 2.2 | 1.3 | 1.5 | 1.6 | 1.0 | 1.8 | 1.6 | 0.4 |
| sCD40L | 1.0 | 2.3 | 1.4 | 0.8 | 1.7 | 1.0 | 1.0 | 0.2 | 1.2 | 0.6 |
| IL-17A | 1.6 | 4.1 | 1.5 | 1.8 | 1.7 | 1.1 | 1.0 | 1.4 | 1.8 | 1.0 |
| IL-1RA | 2.3 | 7.0 | 5.4 | 1.3 | 49.6 | 3.7 | 2.8 | 2.9 | 9.4 | 16.4 |
| IL-1a | 3.1 | 2.5 | 3.8 | 1.2 | 14.1 | 4.2 | 9.1 | 4.3 | 5.3 | 4.2 |
| IL-9 | 1.0 | 1.5 | 1.0 | 1.2 | 1.0 | 1.0 | 1.0 | 1.0 | 1.1 | 0.2 |
| IL-1b | 1.0 | 2.2 | 2.1 | 1.1 | 1.0 | 1.0 | 1.0 | 1.6 | 1.4 | 0.5 |
| IL-2 | 10.9 | 5.7 | 13.6 | 1.8 | 19.7 | 9.2 | 2.5 | 2.7 | 8.3 | 6.3 |
| IL-3 | 1.0 | 1.7 | 1.0 | 1.6 | 1.0 | 1.0 | 1.0 | 1.0 | 1.2 | 0.3 |
| IL-4 | 5.0 | 1.3 | 3.1 | 1.0 | 1.0 | 1.0 | 1.0 | 2.4 | 2.0 | 1.5 |
| IL-5 | 1.0 | 1.6 | 1.0 | 1.2 | 1.3 | 1.1 | 1.0 | 1.0 | 1.1 | 0.2 |
| IL-6 | 12.3 | 6.1 | 12.9 | 1.7 | 57.9 | 3.1 | 4.1 | 8.7 | 13.4 | 18.5 |
| IL-7 | 1.0 | 3.2 | 2.0 | 1.2 | 2.7 | 2.1 | 1.0 | 1.9 | 1.9 | 0.8 |
| IL-8 | 26.9 | 8.0 | 9.9 | 2.3 | 53.6 | 1.8 | 9.1 | 7.8 | 14.9 | 17.4 |
| IP-10 | 3.9 | 10.2 | 17.7 | 3.5 | 15.5 | 6.5 | 4.2 | 10.8 | 9.0 | 5.4 |
| MCP-1 | 18.0 | 27.1 | 49.0 | 6.4 | 23.2 | 4.9 | 3.7 | 8.6 | 17.6 | 15.4 |
| MIP-1a | 3.2 | 2.4 | 1.4 | 1.4 | 6.6 | 2.4 | 1.2 | 2.2 | 2.6 | 1.8 |
| MIP-1b | 3.2 | 2.2 | 2.6 | 1.4 | 6.6 | 2.0 | 2.3 | 2.6 | 2.8 | 1.6 |
| TNFa | 3.4 | 3.5 | 5.0 | 1.5 | 5.2 | 2.2 | 2.4 | 2.4 | 3.2 | 1.3 |
| TNFb | 1.3 | 1.2 | 2.2 | 1.2 | 1.0 | 2.1 | 1.0 | 2.3 | 1.5 | 0.6 |
| VEGF | 2.0 | 6.0 | 1.1 | 1.4 | 27.0 | 1.5 | 1.6 | 1.5 | 5.3 | 8.9 |

The second-to-last column is Mean 6 hr Post dose:Cohort 1.
The last column is SD 6 hr Post dose:Cohort 1.

| | Cohort | | | | | | | | | | | |
|---|---|---|---|---|---|---|---|---|---|---|---|---|
| | 2 | 2 | 2 | 2 | 2 | 2 | 2 | 2 | 2 | 2 | | |
| | Dose | | | | | | | | | | | |
| | 300 | 300 | 300 | 300 | 300 | 300 | 300 | 300 | 300 | 300 | | |
| EGF | 0.9 | 0.9 | 1.5 | 0.7 | 1.6 | 2.0 | 1.6 | ND | 1.0 | ND | 1.3 | 0.5 |
| FGF-2 | 0.9 | 1.0 | 1.3 | 1.1 | 1.2 | 1.1 | 1.2 | ND | 1.3 | ND | 1.1 | 0.1 |
| EOTAXIN | 1.1 | 1.7 | 7.1 | 1.7 | 2.8 | 1.4 | 2.0 | ND | 1.8 | ND | 2.5 | 1.9 |
| TGF-a | 1.2 | 1.8 | 2.5 | 1.0 | 1.4 | 1.4 | 1.3 | ND | 0.7 | ND | 1.4 | 0.5 |
| G-CSF | 1.0 | 7.3 | 2.7 | 1.7 | 1.6 | 1.2 | 1.1 | ND | 2.0 | ND | 2.3 | 2.1 |
| Flt-3L | 0.9 | 1.0 | 1.9 | 1.0 | 0.9 | 1.2 | 1.0 | ND | 1.0 | ND | 1.1 | 0.3 |
| GM-CSF | 1.0 | 1.9 | 1.7 | 1.5 | 1.7 | 1.2 | 1.4 | ND | 1.1 | ND | 1.5 | 0.3 |
| FRACTALKINE | 0.9 | 1.6 | 1.2 | 1.8 | 1.2 | 1.1 | 1.2 | ND | 1.2 | ND | 1.3 | 0.3 |
| IFNa2 | 0.9 | 1.7 | 1.2 | 1.6 | 1.2 | 1.2 | 1.4 | ND | 1.1 | ND | 1.3 | 0.3 |
| IFNg | 1.0 | 1.7 | 1.3 | 2.1 | 1.3 | 1.3 | 1.4 | ND | 0.7 | ND | 1.4 | 0.4 |
| GRO | 0.8 | 0.8 | 1.4 | 0.7 | 3.7 | 1.4 | 2.1 | ND | 1.6 | ND | 1.6 | 1.0 |
| IL-10 | 0.8 | 1.9 | 4.9 | 1.6 | 1.6 | 0.9 | 1.2 | ND | 3.2 | ND | 2.0 | 1.4 |
| MCP-3 | 1.0 | 1.2 | 1.0 | 1.4 | 1.0 | 1.1 | 1.4 | ND | 0.9 | ND | 1.1 | 0.2 |
| IL-12(P40) | 0.9 | 1.1 | 1.4 | 1.9 | 1.0 | 1.2 | 1.1 | ND | 1.0 | ND | 1.2 | 0.3 |
| MDC | 1.0 | 1.0 | 1.4 | 1.0 | 1.0 | 1.0 | 1.0 | ND | 1.1 | ND | 1.1 | 0.2 |
| IL-12P70 | 1.2 | 1.8 | 1.4 | 1.1 | 1.0 | 1.2 | 1.4 | ND | 1.5 | ND | 1.3 | 0.3 |
| IL-13 | 0.9 | 1.1 | 1.0 | 1.0 | 0.9 | 1.1 | 1.6 | ND | 0.8 | ND | 1.1 | 0.3 |
| IL-15 | 0.8 | 1.1 | 2.3 | 1.0 | 1.0 | 1.0 | 1.2 | ND | 1.0 | ND | 1.2 | 0.5 |
| sCD40L | 0.6 | 1.1 | 2.1 | 1.0 | 4.0 | 2.3 | 3.1 | ND | 2.3 | ND | 2.1 | 1.1 |
| IL-17A | 1.0 | 1.9 | 1.2 | 2.0 | 1.1 | 1.1 | 1.2 | ND | 0.9 | ND | 1.3 | 0.4 |
| IL-1RA | 0.9 | 3.0 | 2.1 | 1.6 | 2.3 | 1.3 | 1.4 | ND | 1.3 | ND | 1.7 | 0.7 |
| IL-1a | 1.0 | 1.5 | 2.5 | 4.8 | 1.2 | 1.0 | 1.1 | ND | 1.3 | ND | 1.8 | 1.3 |
| IL-9 | 1.0 | 1.2 | 1.3 | 1.0 | 1.1 | 1.0 | 1.1 | ND | 1.0 | ND | 1.1 | 0.1 |
| IL-1b | 1.0 | 1.2 | 1.6 | 1.0 | 0.9 | 1.0 | 1.1 | ND | 1.0 | ND | 1.1 | 0.2 |
| IL-2 | 1.0 | 5.4 | 16.6 | 1.0 | 2.4 | 1.0 | 1.5 | ND | 2.3 | ND | 3.9 | 5.4 |
| IL-3 | 1.0 | 1.3 | 1.1 | 1.0 | 1.0 | 0.9 | 1.3 | ND | 1.1 | ND | 1.1 | 0.2 |
| IL-4 | 0.9 | 1.3 | 1.1 | 1.0 | 0.9 | 1.1 | 1.6 | ND | 0.9 | ND | 1.1 | 0.2 |
| IL-5 | 1.0 | 1.2 | 1.2 | 1.0 | 1.5 | 1.2 | 1.8 | ND | 1.0 | ND | 1.2 | 0.3 |
| IL-6 | 2.4 | 2.3 | 10.6 | 1.0 | 2.3 | 1.0 | 1.5 | ND | 1.8 | ND | 2.9 | 3.2 |
| IL-7 | 1.0 | 1.8 | 1.6 | 1.6 | 1.4 | 1.7 | 1.5 | ND | 1.4 | ND | 1.5 | 0.3 |
| IL-8 | 1.3 | 6.0 | 3.1 | 5.4 | 3.5 | 1.3 | 3.4 | ND | 1.6 | ND | 3.2 | 1.8 |
| IP-10 | 1.0 | 12.8 | 14.4 | 6.7 | 3.7 | 0.4 | 1.8 | ND | 8.0 | ND | 6.1 | 5.3 |
| MCP-1 | 1.5 | 9.6 | 19.7 | 3.5 | 10.4 | 1.3 | 3.5 | ND | 3.6 | ND | 6.7 | 6.3 |
| MIP-1a | 1.0 | 1.7 | 3.8 | 1.0 | 1.4 | 1.1 | 1.4 | ND | 1.1 | ND | 1.6 | 0.9 |
| MIP-1b | 0.9 | 2.5 | 2.1 | 2.3 | 1.6 | 1.4 | 1.3 | ND | 0.9 | ND | 1.6 | 0.6 |
| TNFa | 1.0 | 3.1 | 3.4 | 1.9 | 1.8 | 1.2 | 1.5 | ND | 1.5 | ND | 1.9 | 0.9 |
| TNFb | 0.9 | 1.1 | 1.1 | 1.0 | 1.0 | 1.3 | 1.7 | ND | 0.9 | ND | 1.1 | 0.3 |
| VEGF | 0.9 | 1.1 | 1.5 | 1.8 | 1.1 | 1.3 | 1.1 | ND | 0.9 | ND | 1.2 | 0.3 |

The second-to-last column is Mean 6 hr Post dose:Cohort 2.
The last column is SD 6 hr Post dose:Cohort 2.

| | Cohort | | | | | | | | |
|---|---|---|---|---|---|---|---|---|---|
| | 7 | 7 | 7 | 7 | 7 | 7 | 7 | | |
| | Dose Nexvax2/ug | | | | | | | | |
| | 150 | 150 | 150 | 150 | 150 | 150 | 150 | | |
| EGF | 0.6 | 1.1 | 2.6 | 2.0 | 2.7 | 0.3 | 1.0 | 1.5 | 1.0 |
| FGF-2 | 0.8 | 1.8 | 1.2 | 1.3 | 1.1 | 1.1 | 0.8 | 1.2 | 0.3 |
| EOTAXIN | 1.5 | 1.7 | 2.2 | 1.2 | 2.0 | 2.7 | 1.9 | 1.9 | 0.5 |
| TGF-a | 1.0 | 1.0 | 1.0 | 1.0 | 0.9 | 1.0 | 1.1 | 1.0 | 0.1 |
| G-CSF | 1.2 | 4.4 | 2.4 | 1.1 | 1.3 | 0.7 | 1.4 | 1.8 | 1.3 |
| Flt-3L | 1.0 | 1.0 | 4.5 | 0.7 | 0.8 | 0.9 | 1.7 | 1.5 | 1.3 |
| GM-CSF | 1.1 | 2.0 | 1.8 | 1.1 | 1.2 | 1.6 | 1.0 | 1.4 | 0.4 |
| FRACTALKINE | 1.1 | 1.9 | 1.4 | 0.9 | 0.9 | 1.7 | 1.3 | 1.3 | 0.4 |
| IFNa2 | 1.1 | 2.5 | 1.7 | 1.2 | 1.1 | 1.5 | 0.8 | 1.4 | 0.6 |
| IFNg | 1.1 | 2.1 | 1.1 | 1.0 | 1.2 | 0.6 | 0.9 | 1.2 | 0.5 |
| GRO | 0.6 | 2.2 | 2.3 | 1.6 | 3.1 | 0.6 | 1.2 | 1.7 | 0.9 |
| IL-10 | 1.2 | 3.6 | 4.4 | 1.1 | 1.0 | 4.8 | 1.1 | 2.5 | 1.7 |
| MCP-3 | 0.8 | 2.1 | 1.9 | 1.1 | 0.9 | 1.5 | 0.9 | 1.3 | 0.5 |
| IL-12(P40) | 0.9 | 2.2 | 1.2 | 1.0 | 1.0 | 1.6 | 1.1 | 1.3 | 0.4 |
| MDC | 1.1 | 1.2 | 1.1 | 1.0 | 1.0 | 0.9 | 1.0 | 1.0 | 0.1 |
| IL-12P70 | 1.2 | 2.3 | 2.0 | 1.0 | 0.9 | 2.1 | 0.7 | 1.5 | 0.7 |
| IL-13 | 1.0 | 1.1 | 1.2 | 1.0 | 1.0 | 1.0 | 0.8 | 1.0 | 0.1 |
| IL-15 | 1.0 | 1.0 | 1.3 | 1.0 | 1.0 | 1.0 | 1.0 | 1.0 | 0.1 |
| sCD40L | 0.5 | 1.4 | 2.3 | 1.9 | 4.3 | 0.2 | 1.3 | 1.7 | 1.4 |
| IL-17A | 1.0 | 1.0 | 1.0 | 1.0 | 1.1 | 0.6 | 0.8 | 0.9 | 0.2 |
| IL-1RA | 1.4 | 3.1 | 2.1 | 1.2 | 1.0 | 1.7 | 1.4 | 1.7 | 0.7 |
| IL-1a | 1.0 | 3.8 | 2.0 | 1.0 | 1.2 | 4.6 | 0.8 | 2.1 | 1.5 |
| IL-9 | 1.0 | 1.0 | 1.0 | 1.0 | 0.9 | 1.0 | 0.8 | 1.0 | 0.1 |
| IL-1b | 1.0 | 1.0 | 1.3 | 1.0 | 0.9 | 1.0 | 1.0 | 1.0 | 0.1 |
| IL-2 | 1.0 | 6.0 | 4.6 | 1.0 | 1.2 | 1.9 | 1.7 | 2.5 | 2.0 |
| IL-3 | 1.0 | 1.0 | 1.0 | 1.0 | 0.9 | 1.0 | 1.2 | 1.0 | 0.1 |
| IL-4 | 1.0 | 2.2 | 1.3 | 0.9 | 0.9 | 1.0 | 1.1 | 1.2 | 0.4 |
| IL-5 | 1.0 | 1.0 | 1.0 | 1.0 | 1.1 | 1.0 | 0.8 | 1.0 | 0.1 |
| IL-6 | 1.0 | 4.5 | 8.5 | 4.4 | 1.8 | 4.3 | 0.9 | 3.6 | 2.7 |
| IL-7 | 1.0 | 1.1 | 1.2 | 1.0 | 1.0 | 1.1 | 0.8 | 1.0 | 0.1 |
| IL-8 | 4.2 | 10.1 | 8.0 | 1.1 | 3.4 | 2.0 | 2.1 | 4.4 | 3.4 |
| IP-10 | 1.5 | 4.6 | 4.6 | 0.9 | 1.1 | 3.6 | 2.9 | 2.7 | 1.6 |
| MCP-1 | 2.1 | 6.5 | 10.2 | 1.3 | 3.8 | 6.0 | 5.5 | 5.1 | 3.0 |
| MIP-1a | 1.0 | 1.2 | 1.7 | 1.0 | 1.2 | 1.4 | 0.9 | 1.2 | 0.3 |
| MIP-1b | 1.2 | 2.4 | 2.6 | 1.0 | 1.3 | 1.9 | 0.9 | 1.6 | 0.7 |
| TNFa | 1.3 | 2.4 | 2.2 | 1.0 | 1.1 | 2.3 | 1.2 | 1.6 | 0.6 |
| TNFb | 1.0 | 1.8 | 1.3 | 1.2 | 1.1 | 1.0 | 1.0 | 1.2 | 0.3 |
| VEGF | 1.0 | 2.0 | 1.2 | 1.1 | 1.2 | 0.7 | 0.9 | 1.2 | 0.4 |

The second-to-last column is Mean 6 hr Post dose:Cohort 7.
The last column is SD 6 hr Post dose:Cohort 7.

| | Cohort | | | | | | | | | | | | | | | |
|---|---|---|---|---|---|---|---|---|---|---|---|---|---|---|---|---|
| | 1 | 1 | 1 | 1 | 2 | 2 | 2 | 7 | 7 | 7 | 7 | 7 | 7 | 7 | | |
| | Dose | | | | | | | | | | | | | | | |
| | 0 | 0 | 0 | 0 | 0 | 0 | 0 | 0 | 0 | 0 | 0 | 0 | 0 | 0 | | |
| EGF | 1.1 | 1.4 | 2.6 | 0.7 | 0.7 | 0.9 | 1.0 | 0.9 | 0.5 | 1.8 | 0.9 | 0.2 | 0.5 | 0.7 | 1.0 | 0.6 |
| FGF-2 | 1.2 | 1.0 | 1.1 | 0.8 | 0.8 | 0.9 | 1.4 | 0.8 | 1.7 | 1.2 | NR | 0.9 | 1.0 | 0.9 | 1.1 | 0.3 |
| EOTAXIN | 1.2 | 1.0 | 1.0 | 1.0 | 1.0 | 0.9 | 1.0 | 0.9 | 0.9 | 0.9 | 0.7 | 1.1 | 1.0 | 0.7 | 1.0 | 0.1 |
| TGF-a | 1.0 | 1.0 | 1.0 | 1.0 | 1.0 | 1.0 | 1.0 | 0.7 | 1.0 | 1.0 | 0.5 | 1.0 | 1.0 | 0.8 | 0.9 | 0.2 |
| G-CSF | 1.4 | 1.2 | 1.1 | 0.9 | 0.9 | 0.9 | 1.1 | 0.9 | 1.0 | 1.1 | 0.6 | 0.8 | 0.8 | 0.8 | 1.0 | 0.2 |
| Flt-3L | 1.0 | 1.0 | 1.0 | 0.9 | 1.0 | 0.8 | 0.9 | 0.7 | 7.1 | 1.0 | 0.4 | 1.0 | 1.0 | 0.7 | 1.3 | 1.7 |
| GM-CSF | 1.4 | 1.1 | 1.1 | 0.9 | 0.7 | 0.9 | 1.4 | 0.9 | 1.5 | 1.1 | NR | 1.0 | 1.0 | 0.7 | 1.1 | 0.3 |
| FRACTALKINE | 1.3 | 0.8 | 1.0 | 0.8 | 0.9 | 0.9 | 1.3 | 0.8 | 1.4 | 1.0 | 0.6 | 0.8 | 1.3 | 0.8 | 1.0 | 0.3 |
| IFNa2 | 1.5 | 1.1 | 1.2 | 0.8 | 0.8 | 0.9 | 1.8 | 0.7 | 1.5 | 1.0 | 0.7 | 1.0 | 1.3 | 0.8 | 1.1 | 0.3 |
| IFNg | 0.9 | 0.9 | 1.3 | 0.9 | 0.6 | 0.9 | 1.1 | 0.9 | 3.0 | 1.0 | 1.0 | 1.2 | 2.4 | 0.8 | 1.2 | 0.7 |
| GRO | 1.6 | 1.2 | 1.4 | 0.9 | 0.9 | 1.1 | 1.2 | 0.7 | 0.3 | 1.2 | 1.4 | 1.1 | 0.5 | 0.6 | 1.0 | 0.4 |
| IL-10 | 1.0 | 1.0 | 0.9 | 0.8 | 1.0 | 1.0 | 1.8 | 0.8 | 1.0 | 1.0 | 0.8 | 1.0 | 1.0 | 0.7 | 1.0 | 0.3 |
| MCP-3 | 1.8 | 1.2 | 1.2 | 1.0 | 0.9 | 1.0 | 1.6 | 0.9 | 1.3 | 1.0 | 0.7 | 1.0 | 1.0 | 0.9 | 1.1 | 0.3 |
| IL-12(P40) | 1.1 | 0.8 | 1.0 | 0.7 | 0.9 | 1.0 | 2.1 | 1.0 | 1.0 | 1.0 | 0.6 | 1.0 | 1.0 | 0.9 | 1.0 | 0.3 |
| MDC | 0.9 | 0.9 | 1.1 | 0.8 | 1.1 | 0.9 | 1.1 | 0.9 | 1.2 | 0.9 | 0.8 | 0.9 | 1.0 | 0.8 | 1.0 | 0.1 |
| IL-12P70 | 1.0 | 1.0 | 1.0 | 0.7 | 0.7 | 1.0 | 1.8 | 0.8 | 4.4 | 1.0 | 0.7 | 1.0 | 1.5 | 0.8 | 1.2 | 1.0 |
| IL-13 | 1.0 | 1.0 | 1.1 | 0.9 | 0.6 | 0.9 | 1.0 | 0.9 | 1.0 | 1.0 | 0.7 | 1.0 | 2.5 | 1.0 | 1.0 | 0.4 |
| IL-15 | 1.0 | 1.0 | 0.8 | 1.0 | 0.9 | 0.9 | 1.0 | 0.9 | 1.0 | 1.0 | 0.7 | 1.0 | 1.0 | 0.8 | 0.9 | 0.1 |
| sCD40L | 1.1 | 1.4 | 1.1 | 1.0 | 0.8 | 1.0 | 1.8 | 0.4 | 0.2 | 1.0 | 1.0 | 0.2 | 0.7 | 1.0 | 0.9 | 0.4 |
| IL-17A | 1.0 | 1.0 | 1.0 | 0.9 | 0.7 | 0.8 | 1.1 | 0.8 | 4.2 | 1.0 | 0.8 | 1.0 | 1.3 | 0.8 | 1.2 | 0.9 |
| IL-1RA | 1.8 | 1.4 | 1.1 | 0.8 | 0.8 | 0.9 | 1.7 | 0.7 | 1.4 | 1.2 | 0.5 | 1.0 | 1.0 | 0.8 | 1.1 | 0.4 |
| IL-1a | 0.8 | 1.0 | 0.9 | 0.9 | 0.9 | 0.9 | 1.2 | 0.9 | 1.1 | 0.6 | 0.5 | 0.6 | 1.0 | 0.8 | 0.9 | 0.2 |
| IL-9 | 1.0 | 1.0 | 1.0 | 1.0 | 0.9 | 0.9 | 1.0 | 0.9 | 1.1 | 1.0 | 0.5 | 1.0 | 1.0 | 0.7 | 0.9 | 0.1 |
| IL-1b | 1.0 | 1.0 | 0.9 | 1.0 | 0.8 | 0.9 | 1.0 | 0.9 | 0.9 | 1.0 | 0.7 | 1.0 | 1.0 | 0.7 | 0.9 | 0.1 |
| IL-2 | 1.0 | 1.0 | 0.9 | 1.0 | 0.9 | 0.9 | 1.0 | 0.9 | 1.2 | 1.0 | 0.6 | 1.0 | 1.0 | 0.8 | 0.9 | 0.1 |
| IL-3 | 1.0 | 1.0 | 1.0 | 1.0 | 1.0 | 0.9 | 1.0 | 0.7 | 1.0 | 1.0 | 1.0 | 1.0 | 1.0 | 0.9 | 1.0 | 0.1 |
| IL-4 | 1.0 | 1.0 | 0.9 | 0.8 | 0.8 | 1.0 | 2.8 | 0.8 | 2.1 | 1.0 | 0.8 | 1.0 | 1.0 | 1.0 | 1.1 | 0.6 |
| IL-5 | 1.0 | 1.0 | 1.0 | 1.0 | 0.8 | 0.9 | 1.0 | 0.6 | 1.9 | 1.0 | 0.4 | 1.0 | 1.0 | 0.9 | 1.0 | 0.3 |
| IL-6 | 1.9 | 1.0 | 0.9 | 1.0 | 0.7 | 1.0 | 1.9 | 0.9 | 1.8 | 1.0 | 0.6 | 1.0 | 1.0 | 1.0 | 1.1 | 0.4 |
| IL-7 | 1.0 | 1.0 | 1.0 | 1.0 | 0.9 | 0.9 | 1.4 | 0.7 | 1.8 | 1.0 | 0.7 | 1.0 | 1.0 | 0.7 | 1.0 | 0.3 |
| IL-8 | 1.0 | 1.0 | 1.4 | 1.1 | 0.8 | 0.9 | 1.0 | 0.9 | 2.1 | 1.1 | 0.8 | 1.1 | 1.0 | 0.8 | 1.1 | 0.3 |
| IP-10 | 0.6 | 0.7 | 0.8 | 0.9 | 1.0 | 0.8 | 0.8 | 0.8 | 1.0 | 0.6 | 0.6 | 0.7 | 0.9 | 0.9 | 0.8 | 0.1 |
| MCP-1 | 1.1 | 0.9 | 0.9 | 0.9 | 0.9 | 0.8 | 0.9 | 0.8 | 0.8 | 1.0 | 1.1 | 0.9 | 1.2 | 0.7 | 0.9 | 0.1 |
| MIP-1a | 1.0 | 1.0 | 1.0 | 1.0 | 0.7 | 1.0 | 1.0 | 0.9 | 1.7 | 1.0 | 0.8 | 1.0 | 1.0 | 0.9 | 1.0 | 0.2 |
| MIP-1b | 1.0 | 1.0 | 1.0 | 0.7 | 0.5 | 0.9 | 1.4 | 0.8 | 2.1 | 1.0 | 0.7 | 1.0 | 1.2 | 0.9 | 1.0 | 0.4 |
| TNFa | 1.0 | 1.0 | 0.9 | 0.9 | 0.9 | 0.9 | 1.1 | 0.9 | 2.1 | 0.9 | 0.6 | 0.9 | 1.0 | 1.1 | 1.0 | 0.3 |
| TNFb | 1.0 | 1.0 | 1.1 | 1.0 | 0.9 | 0.9 | 1.5 | 1.0 | 1.2 | 1.0 | 0.5 | 1.0 | 1.0 | 0.9 | 1.0 | 0.2 |
| VEGF | 3.4 | 0.8 | 1.2 | 0.9 | 0.7 | 0.9 | 1.2 | 1.0 | 2.0 | 1.2 | 0.8 | 1.2 | 1.2 | 1.0 | 1.3 | 0.7 |

The second-to-last column is Mean 6 hr Post dose placebo.
The last column is SD 6 hr Post dose placebo.

### Example 2

### Background

To date, gluten peptide therapies have only been tested in patients on a gluten-free diet for at least 12 months. However, >80% of celiac disease patients are not diagnosed, therefore, determining the minimum time needed on a gluten-free diet to permit peptide therapy responsiveness is needed.

### Objective

To determine the minimum time needed on a gluten-free diet to allow a subject with celiac disease to be responsive to induction of gluten tolerance with a peptide composition.

The peptide composition comprises the following 3 peptides (pE=pyroglutamate):

(SEQ ID NO: 9)
(pE)LQPFPQPELPYPQPQ-amide
(SEQ ID NO: 10)
(pE)QPFPQPEQPFPWQP-amide
(SEQ ID NO: 11)
(pE)PEQPIPEQPQPYPQQ-amide

### Key Inclusion/Exclusion

Newly diagnosed celiac disease patients who are HLA-DQ2.5+/DQ8-; biopsy+ serology proven on a gluten containing diet.

### Key Assessments

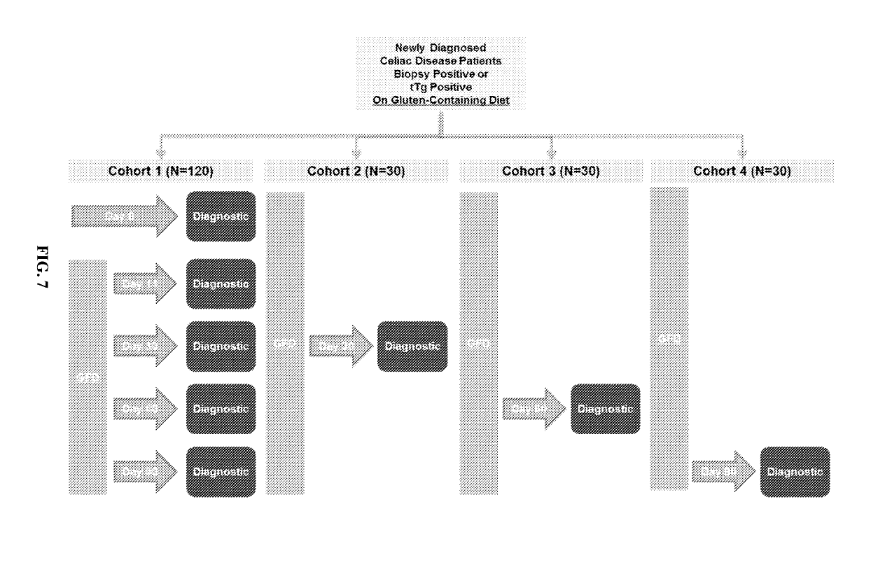

The proportion of patients is tested who respond to the circulating cytokine or chemokine assay described in Example 1 after one of five time points: zero, 14, 30, 60 and 90 days on a gluten-free diet (see FIG. 7). Both serial circulating cytokine assays as well as one time after a fixed time on a gluten-free diet are tested.

### Example 3

### Oral Gluten Challenge Study

### Aim:

To assess cytokines and chemokines circulating in blood during the 6 hours after a cookie was consumed

### Primary Endpoint:

Time-course profile of cytokines and chemokines in serum

### Exploratory Endpoints:

Plasma dynamic profiles of cytokines and chemokines
Profiles of cytokines and chemokines in serum from blood that had been left to stand unseparated for up to 24 hours after collection
Whole blood cytokine release stimulated by gluten and recall antigen peptide mixtures pre-cookie compared to 6 days later

### Subjects:

10 subjects with Celiac disease, biopsy and serology proven, followed GFD for 1 month
Subjects consumed a single gluten-containing cookie containing 3 grams of gluten
Observed for 6 hours: Blood is collected pre-cookie, and then 1 h, 2 h, 3 h, 4 h, 5 h, 6 h post-cookie ingestion (serum/plasma)
Blood was drawn at second visit on day 6
Compared results of oral challenge plasma cytokines with plasma cytokines in post intradermal injection of 150 mcg peptide composition or placebo amongst subjects who are HLA-DQ2.5+ and DQ8− (n=7). The peptide composition comprised the following 3 peptides (pE=pyroglutamate):

(SEQ ID NO: 9)
(pE)LQPFPQPELPYPQPQ-amide
(SEQ ID NO: 10)
(pE)QPFPQPEQPFPWQP-amide
(SEQ ID NO: 11)
(pE)PEQPIPEQPQPYPQQ-amide

### Results

The change in levels of several circulating cytokines were assessed using the MAGPIX® 38plex multiplexing platform and compared at 1-6 Hours after peptide composition intradermal vs. oral gluten challenge. The results are summarized in the table below.

| | Peptide composition | | | | | |
|---|---|---|---|---|---|---|
| | 150 mc i.d. (N = 7) | | | Oral gluten 3 g (N = 7) | | |
| | | Avg. | Peak | | Avg. | Peak |
| Cytokine/ | Rank/ | Fold | Time | Rank/ | Fold | Time |
| Chemokine | 38plex | change | 0-6 hr | 38plex | change | 0-6 hr |
| IL-8 | 1 | 5.3 | 4 hr | 1 | 2.4 | 6 hr |
| MCP-1 | 2 | 5.1 | 6 hr | 5 | 1.6 | 6 hr |
| IL-2 | 3 | 4.7 | 4 hr | 2 | 2.0 | 4 hr |
| IL-6 | 4 | 3.6 | 6 hr | 14 | 1.3 | 6 hr |
| IP-10 | 5 | 2.7 | 6 hr | 3 | 1.9 | 6 hr |
| IL-10 | 6 | 2.5 | 6 hr | 6 | 1.4 | 4 hr |

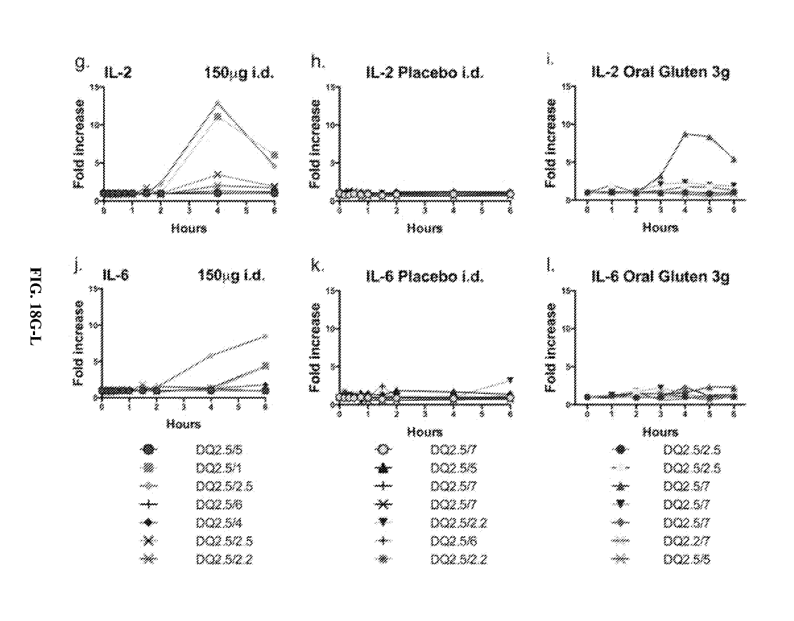

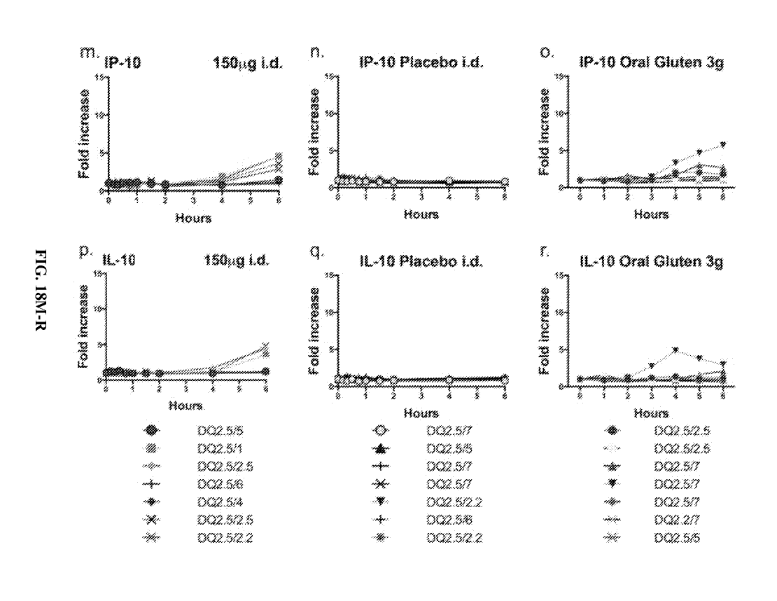

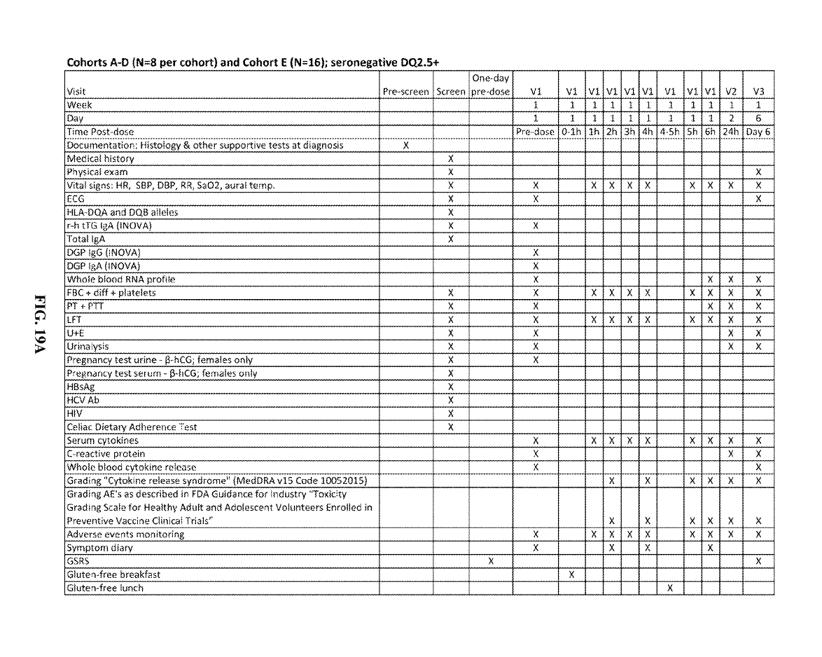

The levels of each cytokine/chemokine measured are shown in FIG. 18 side-by-side for peptide composition intradermal injection, placebo intradermal injection and oral challenge. It was found that oral challenge with a 3 g gluten-cookie bolus was followed by elevated levels of circulating cytokines/chemokines at a similar time and pattern as following peptide composition intradermal injection. The amplitude of cytokine/chemokine elevation following oral gluten (3 g) was less than the peptide composition intradermal injection. The most pronounced elevation in circulating cytokines/chemokines in vivo after injection of the peptide composition or with oral gluten were IL-8 and MCP-1. These results confirm that circulating cytokine/chemokine levels are upregulated by gluten peptide administration and also shown that oral administration is an alternative route of administration from injection for inducing the elevation of the circulating cytokine/chemokine levels.

### Example 4

### Assessment of Circulating Cytokine and Chemokine Levels in Connection with Administration with a Gluten Peptide Composition

### Objectives:

Evaluate the time course of elevation of blood levels of cytokines after administering a single dose of a gluten peptide composition.
Evaluate a tolerable but active dose of the gluten peptide composition that elevates circulating cytokine levels in patients with celiac disease following strict gluten-free diet (GFD).
To test serum levels of circulating cytokines after a selected dose of the gluten peptide composition and correlate with remission status in celiac disease on GFD assessed by pre-dose: tTG IgA, DGP IgG, and DGP IgA serology; whole blood RNA expression profile; distal duodenal (D₂₋₃) VH:CrD ratio, IEL density and tissue RNA expression profile; and Celiac Dietary Adherence Test (CDAT) score.

### Patient Population:

Male or female patients aged 18 to 70 years with celiac disease on GFD.

Key Inclusion Criteria: Patient is between 18 and 70 years old (inclusive). Patient has been diagnosed with celiac disease on the basis of intestinal histology showing villous atrophy according to expert guidelines current at the time of diagnosis, e.g. ESPGHAN 1990(1990). GFD for at least one-month. Patients is positive for HLA-DQA1*05 and/or HLA-DQB1*02 and/or HLA-DQB1*0302 alleles, and if enrolled in Cohorts A-E is positive for both HLA-DQA1*05 and HLA-DQB1*02 (“HLA-DQ2.5+”). Patient is sero-negative for tTG IgA and is not IgA deficient if enrolled in Cohorts A-E.

### Study Design:

At the Screening visit the following will be performed: medical history, physical exam, vital signs (HR, SBP, DBP, RR, SaO₂, aural temperature) and ECG. Screening laboratory tests will include: HLA-DQA and HLA-DQB alleles, recombinant human tTG IgA, total IgA, full blood count with differential and platelet count, clotting, liver and renal function with urinalysis. Subjects will have completed a Celiac Dietary Adherence Test (CDAT) at or within a week of Screening visit. Subjects in Cohort F will also undergo an upper gastrointestinal ndoscopy (EGD) with distal duodenal (D2-3) biopsies for histology to determine D2-3 VH:CrD and IEL density. Treatment Day will be within 6 weeks of the Screening visit. Subjects in Cohorts A-D (n=8 per cohort) and Cohort E (n=16) will be sero-negative for tTG IgA and not IgA deficient, and all will be HLA-DQ2.5+. Each of Cohorts A-D will include two subjects who have no other HLA-DQA allele apart from HLA-DQA1*05 and no other HLA-DQB allele apart from HLA-DQB1*02 (homozygous for HLA-DQ2.5). Cohort E will include four subjects who are homozygous for HLA-DQ2.5. Cohorts A-E will enroll in parallel and assess a single intradermal administration of four dose levels of a gluten peptide composition or placebo (Cohort A: 30 μg, Cohort B: 45 μg, Cohort C: 60 μg, and Cohort D: 150 μg; and Cohort E: placebo) injected in 0.1 mL using a syringe fitted with a West Intradermal Adaptor. A planned interim analysis of data from Cohorts A-E will be performed once these cohorts are completed in order to select one dose level to be compared with placebo in Cohort F (n=30) and Cohort G (n=60). Subjects in Cohorts F and G will enroll in parallel, and within each cohort subjects will be randomized to receive a single intradermal dose of the gluten peptide composition or placebo in a 2:1 ratio. In Cohorts F and G, celiac disease patients will not be required to be sero-negative for tTG IgA, and will possess any of the HLA-DQA and HLA-DQB alleles associated with celiac disease (HLA-DQA1*05, HLA-DQB1*02, and HLA-DQB1*0302) not just HLA-DQ2.5. In all cohorts, planned assessments will be performed pre-dose, 2 h, 4 h, 6 h, 24 h and 6-days after dosing. The schedule of assessments and procedures in Cohorts F and G will be identical to one another except that EGD with D2-3 biopsy will be performed during Screening (before dosing) in Cohort F. Immediately pre-dose and, in some cases, repeatedly during follow-up, laboratory tests will include liver function, full blood examination with differential and platelet count, tTG-IgA, DGP-IgA, DGP-IgG, serum cytokines (including IL-2, IL-6, IL-8, IL-10, TNF-α, IP-10, MCP-1, and eotaxin), whole blood RNA expression profiling, CRP, and gluten peptide-stimulated whole blood IP-10 release. Vital signs will be recorded at the same times as blood is collected for serum cytokine levels. In addition to standard adverse event monitoring, investigators will be required to grade severity of symptoms described in the FDA Guidance for Industry, “Toxicity Grading Scale for Healthy Adult and Adolescent Volunteers Enrolled in Preventive Vaccine Clinical Trials”, and also according to MedDRA v.15 Code 10052015 (“cytokine release syndrome”) at 2 h, 4 h, 6 h, 24 h and 6-days after dosing. Subjects will also complete a symptom diary before, and when cytokine levels are assessed. The GSRS will be completed pre-dose and on day-6 post-dose relating to symptoms experienced during the prior one week.

### Pharmacodynamic Assessments:

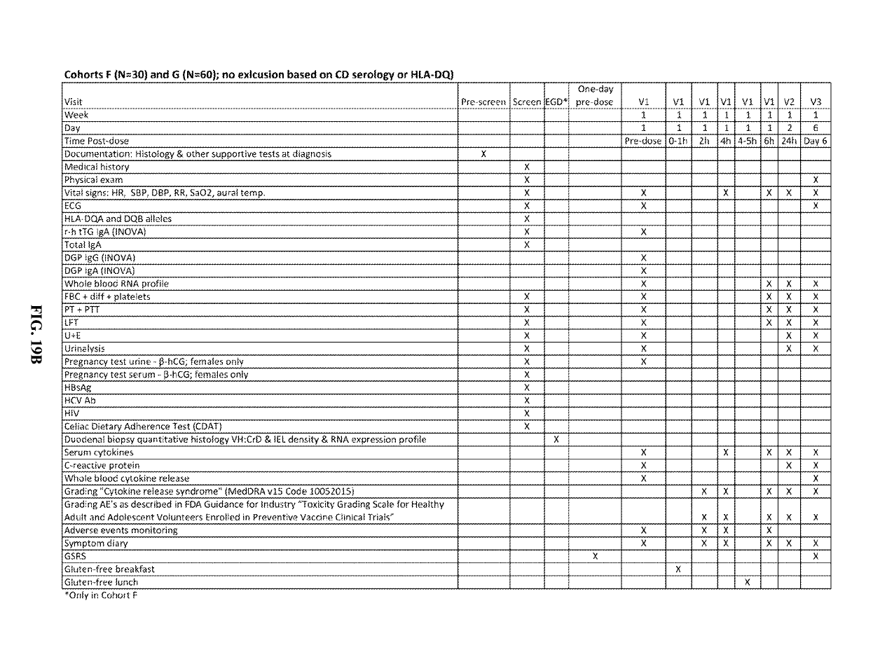

Serum cytokines including IL-2, IL-6, IL-8, IL-10, TNF-α, IP-10, MCP-1, and eotaxin, whole blood RNA expression profiling, CRP, and gluten-peptide-stimulated whole blood IP-10 release will be assessed at the time points outlined in the Schedule of Assessments (SOA, see FIGS. 19A and 19B).

### Study Drug, Dosage, and Route of Administration:

The gluten peptide composition administered includes 3 peptides in equimolar amounts in sodium chloride 0.9% USP: ELQPFPQPELPYPQPQ (SEQ ID NO: 9), EQPFPQPEQPFPWQP (SEQ ID NO: 10), and EPEQPIPEQPQPYPQQ (SEQ ID NO: 11). For each peptide in the composition, the N-terminal glutamate is a pyroglutamate and the carboxyl group of the C-terminal proline or glutamine is amidated.

Dosage regimens planned:

Cohort A: 30 μg n=8

Cohort B: 45 μg n=8

Cohort C: 60 μg n=8

Cohort D: 150 μg n=8

Cohort E: Placebo n=16

Cohort F: dose selected from 30, 45, 60 or 150 μg N=30

Cohort G: dose same as Cohort F, N=60

Dose frequency: single dose

Route of administration: intradermal injection using a West Intradermal Adaptor fitted to a fixed needle 1-mL insulin syringe

### Sample Size:

Approximately 138 patients

### Statistical Methods:

(1) Grading and duration of cytokine release syndrome and self-reported symptoms after administration of 30 μg, 45 μg, 60 μg, or 150 μg of gluten peptide composition.

(2) Maximal elevation in the serum concentration of a cytokine including IL-2, IL-6, IL-8, IL-10, TNF-α, IP-10, MCP-1, eotaxin, or CRP from pre-dose levels.

(3) Maximal serum concentrations of cytokines including IL-2, IL-6, IL-8, IL-10, TNF-α, IP-10, MCP-1, eotaxin, or CRP and correlate with the timing and severity of the cytokine release syndrome (MedDRA v.15 Code 10052015) and reported symptoms after administering a single dose of the gluten peptide composition.

(4) Maximal serum concentrations and AUC analysis of cytokines including IL-2, IL-6, IL-8, IL-10, TNF-α, IP-10, MCP-1, eotaxin, or CRP, severity of the cytokine release syndrome (MedDRA v.15 Code 10052015) and reported symptoms after administering a single dose of the gluten peptide composition and correlate with: (a) pre-dose remission status assessed by reported compliance with gluten free diet (CDAT score), (b) pre-dose serum levels of tTG IgA, DGP IgG, and/or DGP IgA (c) pre-dose distal duodenal villous height-crypt depth ratio, IEL density, and/or distal duodenal RNA expression profile, (d) pre-dose whole blood RNA expression profile, and (e) being homozygous for HLA-DQA1*05 and/or HLA-DQB1*02 alleles.

Cytokine concentrations will be analyzed as an absolute value (pg/mL), as a delta value compared to pre-dose levels (pg/mL), and as a ratio to pre-dose levels.

Data from each of the Cohorts A-D will be compared with Cohort E (placebo-treated) in the planned interim analysis.

In the final analysis of Cohorts F and G, placebo-treated subjects will be compared with composition-treated subjects.

Adverse events will be summarized for each dosing cohort, presenting the numbers and percent of patients having any adverse event (AE) and having AEs in each system organ class (SOC) and preferred term.

### Rationale:

This study is designed to optimize biomarker selection and establish a minimal dose of the gluten peptide composition that provokes a readily detectable increase in the biomarker. Dose ranging in Cohorts A-D will provide a single dose level to be studied in larger numbers of subjects in Cohort F and G. The current study will also provide greater understanding of factors that may influence or correlate with the magnitude of cytokine elevations and change in gene expression profiles in blood as well as clinical responses following a single dose of the gluten peptide composition: prior gluten exposure, compliance with GFD, intestinal tissue injury assessed by quantitative histology and gene expression profile, and pharmacogenetics. The highest dose level to be assessed in the current study is 150 μg and the lowest dose level to be assessed is 30 μg. Subjects in the current study will be monitored closely during the first six hours after dosing as this has been found to be when cytokine elevations and symptoms are most pronounced. Clinical review and further blood tests the following day and six days later will establish when normalization of cytokines occurs.

### Study Design (Further Details):

The first phase of the study (Cohorts A-E) is a double-blind dose ranging study to evaluate the immunological and clinical response to a single intra-dermal dose of the gluten peptide composition in HLA-DQ2.5+ patients with celiac disease on a GFD who are sero-negative for tTG IgA and not IgA deficient. The second phase of the study (Cohorts F and G) is a double-blind study evaluating the immunological and clinical response to a single intra-dermal fixed dose of the gluten peptide composition in patients with celiac disease on a GFD to correlate response with reported compliance with GFD, serum levels of tTG IgA, DGP IgG, and DGP IgA, intestinal injury measured by quantitative histology and by duodenal gene expression.

### Cohorts A-E

The study will consist of a Screening Period lasting up to six weeks followed by a one-day Treatment Period, and a 6-day Follow-up Period. The study will include four visits (one at Screening, one on the treatment day, and two during follow-up). Prior to screening, copies of medical reports shall be collected confirming intestinal villous atrophy and other laboratory and clinical abnormalities present when celiac disease was first diagnosed. At or within one week prior to the Screening visit, a self-administered dietary survey (Celiac Dietary Adherence Test, CDAT) will be completed to assess compliance with GFD (Leffler, Dennis et al. 2009). At the Screening visit, a medical history will be taken and a physical examination performed. Vital signs including HR, SBP, DBP, RR, SaO₂, and aural temperature will be recorded. An ECG will also be performed. At Screening, blood will be collected for the following: HLA-DQ genotyping to test for a panel of HLA-DQA and HLA-DQB alleles, recombinant human tTG IgA, and total IgA, full blood count with differential and platelet count, clotting, liver and renal function. Urinalysis will also be performed. Patients meeting the entry criteria, who are also HLA-DQ2.5+ and sero-negative for tTG IgA and not IgA deficient will be randomized to receive a single intra-dermal dose of the gluten peptide composition: 30 μg (Cohort A), 45 μg (Cohort B), 60 μg (Cohort C), or 150 μg (Cohort D) or placebo (Cohort E). There will be eight subjects enrolled in each of Cohorts A-D, and sixteen in Cohort E. Randomization of subjects in Cohorts A-E will account for the requirement that two subjects in each of Cohort A-D and four subjects in Cohort E will have demonstrated no other HLA-DQA allele apart from HLA-DQA1*05 and no other HLA-DQB allele apart from HLA-DQB1*02 (“homozygous for HLA-DQ2.5”).

The day before treatment day, subjects will complete the GSRS according to the symptoms experienced during the prior one week. On the treatment day, subjects will arrive before 9 am after an overnight fast (no breakfast). The symptom diary applying to the previous 2 h will be completed prior to vital signs including HR, SBP, DBP, RR, SaO₂, and aural temperature being recorded. An ECG will also be performed. An 18-gauge intra-venous cannula will be inserted, preferably into a large antecubital vein. A 3-way tap will be attached and flushed with 2.5 mL of sterile normal saline containing heparin 10 U/mL. A loose tourniquet may be applied if necessary to facilitate blood collection, which will be preceded by withdrawing and discarding a volume of 2.5 mL. Blood is then collected via an adaptor directly into appropriate tubes for full blood count with differential and platelet count, clotting, liver and renal function, serum tTG-IgA, DGP-IgA, DGP-IgG, serum cytokines, CRP, whole blood RNA expression, and gluten peptide-stimulated whole blood cytokine release. After blood collection, the cannula will be flushed with 2.5 mL of sterile normal saline containing heparin 10 U/mL.

Gluten peptide composition or placebo will be administered in 0.1 mL using a syringe fitted with a West Intradermal Adaptor.

During the six hours after dosing vital signs (HR, SBP, DBP, RR, SaO₂, aural temperature) will be recorded, and blood will be collected hourly as described in the SOA (FIGS. 19A and 19B). Adverse events will be recorded after dosing. At 2 h, 4 h, and 6 h after dosing the symptom diary will be completed and apply to symptoms experienced in the previous two-hours, and a grading (0-5) for the severity of “Cytokine release syndrome” (MedDRA v.15 Code 10052015) since dosing will be recorded (see Appendix: Cytokine Release Syndrome Defined by Common Terminology Criteria for Adverse Events v4.03). After the assessments at 6 hours post-dose, the intra-venous cannula will be removed and the subject discharged from the study site.

The following day, subjects will return to the study site for assessments 23-25 h after dosing as described in the SOA.

The End-of-Study visit will be six days after dosing. Subjects will return to the study site for assessments as described in the SOA. A Gastrointestinal Symptom Rating Scale (GSRS) will be completed according the symptoms experienced during the prior one week since dosing.

### Cohorts F and G

After analysis of clinical and laboratory data derived from completed Cohorts A-E, a single dose level of the gluten peptide composition will be chosen and compared with placebo in Cohorts F and G. Subjects will be given the option to select whether they wish to enroll in Cohort E or Cohort F. The ratio of subjects randomized to receive the gluten peptide composition or placebo will be 2:1.

Subjects enrolled in Cohorts F and G will meet the same entry criteria as Cohorts A-E except that they will not be required to be sero-negative for tTG IgA, and will possess any of the HLA-DQA and HLA-DQB alleles associated with celiac disease (HLA-DQA1*05, HLA-DQB1*02, and HLA-DQB1*0302) not just HLA-DQ2.5.

Subjects may be enrolled in Cohorts F and G after being re-screened if they were excluded from enrolling in Cohorts A-E because tTG IgA serology was elevated, or because of IgA deficiency, or because they were not HLA-DQ2.5+, or because there was no further requirement in these cohorts for patients homozygous for HLA-DQ2.5 or for patients who were not homozygous for HLA-DQ2.5.

The Screening Visit for subjects in Cohorts F and G will be the same as for Cohorts A-E. Subjects who were previously screened for Cohorts A-E do not require repeat HLA-DQ genotyping, but all other screening tests and procedures will be repeated.

During the month prior to the treatment day, subjects in Cohort F will undergo an EGD for collection of six biopsies from the 2nd and 3rd parts of the duodenum. Quantitative histology will be performed on formalin-fixed biopsies to assess VH:CrD ratio and IEL density, and two biopsies will be collected into appropriate media for subsequent RNA expression profiling.

Procedures and assessments for Cohorts F and G will be otherwise the same as Cohorts A-E, except that vital signs, symptom diary, grading symptoms according to MedDRA v.15 Code 10052015 “cytokine release syndrome” and the FDA Guidance for Industry, “Toxicity Grading Scale for Healthy Adult and Adolescent Volunteers Enrolled in Preventive Vaccine Clinical Trials”, and blood collection will be limited to 4 h, 5 h, and 6 h after dosing.

### Clinical Laboratory Assessments:

Clinical laboratory tests (hematology and biochemistry) will be taken at several time points during the trial per the following schedule:
Screening
Baseline (Treatment Day, pre-dose)
During the 6 hours after dosing
Day 2
Day 6

Urinalysis will be performed via dipstick and a microscopic exam performed only if needed, depending on the result of the dipstick. Urine samples for urinalysis will be obtained at:
Screening
Baseline (Treatment Day, pre-dose)
Day 2
Day 6 (EOS)

Screening samples will be obtained under fasting conditions (no food or drink, except water, for at least 8 hours before sample collection). The laboratory tests to be performed are presented in the table below.

Clinical Laboratory Tests

| Category | Parameters |
|---|---|
| Hematology | Red blood cell (RBC) count, hemoglobin (Hb), |
| | hematocrit (Hct), platelets, and white blood cell (WBC) |
| | count with differential (bands, neutrophils, lymphocytes, |
| | monocytes, eosinophils, basophils) and platelet count |
| Coagulation | Prothrombin time (PT), and partial throboplastin time |
| | (PTT) |
| Chemistry | |
| Electrolytes | Sodium, potassium, chloride, bicarbonate (CO2) |
| Liver function | alkaline phosphatase, aspartate aminotransferase (AST), |
| tests | alanine aminotransferase (ALT), total bilirubin, direct |
| | bilirubin |
| Renal function | Blood urea/blood urea nitrogen (BUN), creatinine |
| parameters | |
| Other | Albumin, total protein, globulin, C-Reactive protein |
| | (CRP), and total serum IgA |
| Urinalysis | Urinalysis will be performed via dipstick and a |
| | microscopic exam performed only if needed, depending |
| | on the result of the dipstick. |
| Pregnancy | Serum and urine beta human chorionic gonadotropin (β- |
| Tests | hCG) for all female subjects |
| Viral Tests | Serum Hepatitis B (HBsAg), hepatitis C (HCV Ab), and |
| | human immunodeficiency virus (HIV) |
| Celiac Disease | Serum recombinant human transglutaminase IgA, |
| Serology | deamidated gliadin peptide IgG and deamidated gliadin |
| | peptide IgA |
| Cytokine Tests | Serum IL-2, IL-6, IL-8, IL-10, TNF-α, IP-10, MCP-1, |
| | eotaxin and additional cytokines and chemokines |
| | according to multiplex panels available |
| Genetic Tests | HLA-DQA and HLA-DQB allele analysis by |
| | polymerase chain reaction |
| Gene | Gene expression profiles will be determined by reverse |
| Expression | transcription and amplification of RNA in whole blood |
| Tests | or intestinal tissue collected into dedicated tubes |
| | containing an RNA-inhibitor. |

### Further Study Drug Information:

The gluten peptide compositions is provided as a 1.5 mg/mL stock and is supplied in sterile 2 ml vials for single-use with a concentration of each of the individual peptides at 0.5 mg/ml dissolved in sterile 0.9% sodium chloride. Vials are provided frozen and will be allowed to thaw at room temperature immediately prior to being prepared for administration to study subjects. The final injection volume of each syringe is 0.1 mL (100 μL). Sterile 0.9% sodium chloride for injection will be used to dilute the stock 1.5 mg/mL to the appropriate concentration for administration, and will also be used to prepare the placebo (0.9% sodium chloride).

The doses used are: 30 μg, 45 μg, 60 μg or 150 μg in syringes. Each patient will receive one intradermal dose containing the same dose volume (100 μL). To make each of the compositions, the following will be performed: 20 μL from the stock vial diluted with 80 μL 0.9% saline for the 30 μg dose; 30 μL from the stock vial diluted with 70 μL 0.9% saline for the 45 μg dose; 40 μL of from the stock vial diluted with 60 μL 0.9% saline for the 60 μg dose. For the 150 μg dose, 100 μL will be drawn from the stock 1.5 mg/mL vial. The placebo will be 100 μL 0.9% saline. The West Intradermal Adaptor will be fitted to the syringe immediately before the injection is administered to the patient.

### Primary Endpoints

At Interim Analysis after Completion of Cohorts A-E:

Grading and frequency of adverse events (AE's) after dosing with 30 μg (Cohort A), 45 μg (Cohort B), 60 μg (Cohort C) of the gluten peptide composition compared with 150 μg (Cohort D) of the gluten peptide composition and with placebo (Cohort E).

Proportion of subjects receiving a dose of the gluten peptide composition that does not cause AE's with severity graded as any more than moderate and causes elevation of serum cytokines including IL-2, IL-6, IL-8, IL-10, TNF-α, IP-10, eotaxin and MCP-1 or CRP greater than any subject dosed with placebo (Cohort E).

At Final Analysis after Completion of Cohorts F and G:

Grading and frequency of AE's in subjects administered a specified dose of the peptide composition compared to subjects dosed with placebo in Cohorts G and F.

Proportion of HLA-DQ2.5+ subjects who are in remission as assessed by serum levels of tTG IgA, DGP-IgA, or DGP-IgG, or quantitative histology of distal duodenal biopsies, or who closely adhere to GFD (CDAT score), administered a specified dose of the peptide composition that elevates serum cytokines including IL-2, IL-6, IL-8, IL-10, TNF-α, IP-10, eotaxin and MCP-1 or CRP at pre-specified optimal time-points to levels greater than any subject dosed with placebo in Cohorts G and F.

### Secondary Endpoints

Grading and frequency of AE's in subjects administered a specified dose of the gluten peptide composition in Cohorts G and F according to whether they are HLA-DQ2.5+ and homozygotes or heterozygotes for HLA-DQA1*05 and/or HLA-DQB1*02. Maximal elevations of serum cytokines including IL-2, IL-6, IL-8, IL-10, TNF-α, IP-10, eotaxin and MCP-1 or CRP in subjects administered a specified dose of the gluten peptide composition in Cohorts G and F according to whether they are HLA-DQ2.5+ and homozygotes or heterozygotes for HLA-DQA1*05 and/or HLA-DQB1*02. Gene expression profile in pre-dose duodenal biopsies compared to changes in serum cytokines including IL-2, IL-6, IL-8, IL-10, TNF-α, IP-10, eotaxin and MCP-1 or CRP at serial time points after dosing with the gluten peptide composition compared to placebo.

### REFERENCE LIST

(1990). “Revised criteria for diagnosis of coeliac disease. Report of Working Group of European Society of Paediatric Gastroenterology and Nutrition.” Arch Dis Child 65(8): 909-911.
(2007). Guidance for Industry. Toxicity Grading Scale for Healthy Adult and Adolescent Volunteers Enrolled in Preventive Vaccine Clinical Trials. U. S. D. o. H. a. H. Services. Rockville Md. U.S.A.
(2010). Common Terminology Criteria for Adverse Events (CTCAE) Version 4.03. U. S. D. O. H. A. H. SERVICES.
Anderson, R. P. (2005). “Coeliac disease.” Aust Fam Physician 34(4): 239-242.
Anderson, R. P., P. Degano, A. J. Godkin, D. P. Jewell and A. V. Hill (2000). “In vivo antigen challenge in celiac disease identifies a single transglutaminase-modified peptide as the dominant A-gliadin T-cell epitope.” Nat Med 6(3): 337-342.

Anderson, R. P., M. J. Henry, R. Taylor, E. L. Duncan, P. Danoy, M. J. Costa, K. Addison, J. A. Tye-Din, M. A. Kotowicz, R. E. Knight, W. Pollock, G. C. Nicholson, B. H. Toh, M. A. Brown and J. A. Pasco (2013). “A novel serogenetic approach determines the community prevalence of celiac disease and informs improved diagnostic pathways.” BMC Med 11(1): 188.
Bardella, M. T., P. Velio, B. M. Cesana, L. Prampolini, G. Casella, C. Di Bella, A. Lanzini, M. Gambarotti, G. Bassotti and V. Villanacci (2007). “Coeliac disease: a histological follow-up study.” Histopathology 50(4): 465-471.
Bodd, M., C. Y. Kim, K. E. Lundin and L. M. Sollid (2012). “T-cell response to gluten in patients with HLA-DQ2.2 reveals requirement of peptide-MHC stability in celiac disease.” Gastroenterology 142(3): 552-561.
Brottveit, M., M. Raki, E. Bergseng, L. E. Fallang, B. Simonsen, A. Lovik, S. Larsen, E. M. Loberg, F. L. Jahnsen, L. M. Sollid and K. E. Lundin (2011). “Assessing possible celiac disease by an HLA-DQ2-gliadin Tetramer Test.” Am J Gastroenterol 106(7): 1318-1324.
Catassi, C., R. P. Anderson, I. D. Hill, S. Koletzko, E. Lionetti, N. Mouane, M. Schumann and S. K. Yachha (2012). “World perspective on celiac disease.” J Pediatr Gastroenterol Nutr 55(5): 494-499.
Chen, Z., N. Dudek, O. Wijburg, R. Strugnell, L. Brown, G. Deliyannis, D. Jackson, F. Koentgen, T. Gordon and J. McCluskey (2002). “A 320-kilobase artificial chromosome encoding the human HLA DR3-DQ2 MHC haplotype confers HLA restriction in transgenic mice.” J Immunol 168(6): 3050-3056.
de Kauwe, A. L., Z. Chen, R. P. Anderson, C. L. Keech, J. D. Price, O. Wijburg, D. C. Jackson, J. Ladhams, J. Allison and J. McCluskey (2009). “Resistance to celiac disease in humanized HLA-DR3-DQ2-transgenic mice expressing specific anti-gliadin CD4+ T cells.” J Immunol 182(12): 7440-7450.
Gianfrani, C., R. Troncone, P. Mugione, E. Cosentini, M. De Pascale, C. Faruolo, S. Senger, G. Terrazzano, S. Southwood, S. Auricchio and A. Sette (2003). “Celiac disease association with CD8+ T cell responses: identification of a novel gliadin-derived HLA-A2-restricted epitope.” J Immunol 170(5): 2719-2726.
Henderson, K. N., J. A. Tye-Din, H. H. Reid, Z. Chen, N. A. Borg, T. Beissbarth, A. Tatham, S. I. Mannering, A. W. Purcell, N. L. Dudek, D. A. van Heel, J. McCluskey, J. Rossjohn and R. P. Anderson (2007). “A structural and immunological basis for the role of human leukocyte antigen DQ8 in celiac disease.” Immunity 27(1): 23-34.
Jabri, B. and L. M. Sollid (2009). “Tissue-mediated control of immunopathology in coeliac disease.” Nat Rev Immunol 9(12): 858-870.
Karell, K., A. S. Louka, S. J. Moodie, H. Ascher, F. Clot, L. Greco, P. J. Ciclitira, L. M. Sollid, J. Partanen and D. European Genetics Cluster on Celiac (2003). “HLA types in celiac disease patients not carrying the DQA1*05-DQB1*02 (DQ2) heterodimer: results from the European Genetics Cluster on Celiac Disease.” Hum Immunol 64(4): 469-477.
Kaukinen, K., M. Peraaho, K. Lindfors, J. Partanen, N. Woolley, P. Pikkarainen, A. L. Karvonen, T. Laasanen, H. Sievanen, M. Maki and P. Collin (2007). “Persistent small bowel mucosal villous atrophy without symptoms in coeliac disease.” Aliment Pharmacol Ther 25(10): 1237-1245.
Keech, C. L., Mcluskey, J. (2008). Bioactivity assessment of Nexvax2 in a relevant in vivo mouse model: dose response, University of Melbourne.
Keech, C. L., Mcluskey, J. (2008). Bioactivity assessment of Nexvax2 in a relevant in vivo mouse model: immune modulation following repeat administration of Nexvax2, University of Melbourne.
Keech, C. L., Mcluskey, J. (2008). Bioactivity assessment of Nexvax2 in a relevant in vivo mouse model: response of antigen primed gliadin specific T cells., University of Melbourne.
Keech, C. L., Mcluskey, J. (2008). Bioactivity assessment of Nexvax2 in a relevant in vivo mouse model: response of naïve gliadin specific T cells., University of Melbourne.
Keech, C. L., Mcluskey, J. (2010). Bioactivity assessment of Nexvax2 in a relevant in vivo mouse model, University of Melbourne.
Keech, C. L., Mcluskey, J. (2011). Bioactivity assessment of Nexvax2 in a relevant in vivo mouse model., University of Melbourne.
Koskinen, L., J. Romanos, K. Kaukinen, K. Mustalahti, I. Korponay-Szabo, D. Barisani, M. T. Bardella, F. Ziberna, S. Vatta, G. Szeles, Z. Pocsai, K. Karell, K. Haimila, R. Adany, T. Not, A. Ventura, M. Maki, J. Partanen, C. Wijmenga and P. Saavalainen (2009). “Cost-effective HLA typing with tagging SNPs predicts celiac disease risk haplotypes in the Finnish, Hungarian, and Italian populations.” Immunogenetics 61(4): 247-256.
Leffler, D., D. Schuppan, K. Pallav, R. Najarian, J. D. Goldsmith, J. Hansen, T. Kabbani, M. Dennis and C. P. Kelly (2013). “Kinetics of the histological, serological and symptomatic responses to gluten challenge in adults with coeliac disease.” Gut 62(7): 996-1004.
Leffler, D. A., M. Dennis, J. B. Edwards George, S. Jamma, S. Magge, E. F. Cook, D. Schuppan and C. P. Kelly (2009). “A simple validated gluten-free diet adherence survey for adults with celiac disease.” Clin Gastroenterol Hepatol 7(5): 530-536, 536 e531-532.
Liu, E., H. S. Lee, C. A. Aronsson, W. A. Hagopian, S. Koletzko, M. J. Rewers, G. S. Eisenbarth, P. J. Bingley, E. Bonifacio, V. Simell, D. Agardh and T. S. Group (2014). “Risk of pediatric celiac disease according to HLA haplotype and country.” N Engl J Med 371(1): 42-49.
Murray, J. A., T. Watson, B. Clearman and F. Mitros (2004). “Effect of a gluten-free diet on gastrointestinal symptoms in celiac disease.” Am J Clin Nutr 79(4): 669-673.
Ontiveros, N., J. A. Tye-Din, M. Y. Hardy and R. P. Anderson (2014). “Ex-vivo whole blood secretion of interferon (IFN)-gamma and IFN-gamma-inducible protein-10 measured by enzyme-linked immunosorbent assay are as sensitive as IFN-gamma enzyme-linked immunospot for the detection of gluten-reactive T cells in human leucocyte antigen (HLA)-DQ2.5(+)-associated coeliac disease.” Clin Exp Immunol 175(2): 305-315.
Raki, M., L. E. Fallang, M. Brottveit, E. Bergseng, H. Quarsten, K. E. Lundin and L. M. Sollid (2007). “Tetramer visualization of gut-homing gluten-specific T cells in the peripheral blood of celiac disease patients.” Proc Natl Acad Sci USA. 104(8):2831-6.
Simell, S., S. Hoppu, A. Hekkala, T. Simell, M. R. Stahlberg, M. Viander, H. Yrjanainen, J. Gronlund, P. Markula, V. Simell, M. Knip, J. Ilonen, H. Hyoty and O. Simell (2007). “Fate of five celiac disease-associated antibodies during normal diet in genetically at-risk children observed from birth in a natural history study.” Am J Gastroenterol 102(9): 2026-2035.
Simell, S., A. Kupila, S. Hoppu, A. Hekkala, T. Simell, M. R. Stahlberg, M. Viander, T. Hurme, M. Knip, J. Ilonen, H. Hyoty and O. Simell (2005). “Natural history of transglutaminase autoantibodies and mucosal changes in children carrying HLA-conferred celiac disease susceptibility.” Scand J Gastroenterol 40(10): 1182-1191.
Sollid, L. M., S. W. Qiao, R. P. Anderson, C. Gianfrani and F. Koning (2012). “Nomenclature and listing of celiac disease relevant gluten T-cell epitopes restricted by HLA-DQ molecules.” Immunogenetics 64(6): 455-460.
Tye-Din, J. A., R. P. Anderson, R. A. Ffrench, G. J. Brown, P. Hodsman, M. Siegel, W. Botwick and R. Shreeniwas (2010). “The effects of ALV003 pre-digestion of gluten on immune response and symptoms in celiac disease in vivo.” Clin Immunol 134(3): 289-295.
Vader, W., D. Stepniak, Y. Kooy, L. Mearin, A. Thompson, J. J. van Rood, L. Spaenij and F. Koning (2003). “The HLA-DQ2 gene dose effect in celiac disease is directly related to the magnitude and breadth of gluten-specific T cell responses.” Proc Natl Acad Sci USA 100(21): 12390-12395.

### Example 5

### Further Quantitative Analysis of Prominent Cytokines and Chemokines Identified in Example 1

Plasma levels of IL-2, IL-8, and MCP-1 exhibited the greatest relative elevations from pre-dose levels after the first administration of the peptide composition. During the 6-hour period after administration of the first dose of the peptide composition, peak plasma concentrations for IL-2 and IL-8 were observed at 4 hours, but other cytokines and chemokines that were commonly elevated generally peaked at 6 hours or plateaued between 4 and 6 hours (see below table).

Median Peak Plasma IL-8, IL-2, and MCP-1 Concentrations after the First and Last Doses of Peptide Composition

| | | | | Median | Median |
|---|---|---|---|---|---|
| | | | | Plasma | Plasma |
| | | | | Concentration | Concentration |
| | | | Peak | After | After |
| | | | Median | Dosing With | Dosing With |
| | | | Fold | peptide | peptide |
| | | | Change | composition | composition |
| | | Hours | from | (pg/mL) | (pg/mL) |
| | | Post- | Pre-dose | First Dose | Last Dose |
| Cytokine/Chemokine | Cohort | Dose | (Day 1) | (Day 1) | (Day 53) |
| IL-8 | 1 | 4 | 32 | 400** | 13 |
| | 2 | 4 | 3 | 105** | 4 |
| | 7 | 4 | 4 | 45* | 7 |
| IL-2 | 1 | 4 | 26 | 103** | 5 |
| | 2 | 4 | 2 | 67 | 3 |
| | 7 | 4 | 2 | 20 | 7 |
| MCP-1 | 1 | 6 | 15 | 2276** | 249 |
| | 2 | 4 | 4 | 767** | 185 |
| | 7 | 6 | 6 | 962** | 216 |

*Concentration significantly different than pre-dose (p < 0.05)
**Concentration significantly different than pre-dose (p < 0.01)

IL-2 and IL-8 were significantly elevated above pre-dose levels by 2 hours after dosing, while several other cytokines and chemokines that showed less substantial elevations were significantly different from pre-dose concentrations as early as 1.5 hours after dosing. As compared to the peak concentrations on Day 1, median concentrations of IL-8, IL-2, and MCP-1 were substantially lower in all Cohorts after the last dose on Day 53. After the first dose of the peptide composition, the highest median increase in plasma concentrations of any cytokine or chemokine compared to pre-dose levels was observed for IL-8. In Cohort 1, peak IL-8 levels were 32-fold higher than pre-dose levels, representing a greater fold change than for either of the other 2 cohorts. However, the median pre-dose plasma level of IL-8 and several other cytokines and chemokines were lower in Cohort 1 than the other 2 cohorts. In Cohort 1, median plasma concentrations of IL-8 increased from 8 pg/mL to a peak of 339 pg/mL. The highest observed plasma IL-8 concentrations after the first dose of the peptide composition were pre-dose: 14 pg/mL, 4 hours: 701 pg/mL and pre-dose: 3 pg/mL, 4 hours: 767 pg/mL, both from subject who were in Cohort 1. In contrast to the increases seen after the first dose of the peptide composition, median plasma concentrations of IL-8 after the final dose of the peptide composition in Cohorts 1 and 7 were essentially unchanged from pre-dose levels (13 pg/mL at 4 hours versus 13 pg/mL pre-dose in Cohort 1; and 7 pg/mL at 4 hours versus median of 6 pg/mL pre-dose in Cohort 7) (see table above). Median peak plasma concentration of MCP-1 was 3362 pg/mL higher than pre-dose levels (181 pg/mL) in Cohort 1. In Cohort 2, median peak plasma concentration of MCP-1 was 483 pg/mL greater than median pre-dose levels (200 pg/mL), and in Cohort 7 median peak plasma concentration of MCP-1 was 815 pg/mL higher than pre-dose (170 pg/mL). Plasma MCP-1 concentrations increased by over 5000 pg/mL after the first dose of the peptide composition in 2 subjects (one subject in Cohort 1, pre-dose: 313 pg/mL, 6 hours: 5626 pg/mL; and another subject in Cohort 2, pre-dose: 310 pg/mL, 4 hours: 5368 pg/mL). After the final dose of the peptide composition, median plasma concentrations of MCP-1 at 6 hours were relatively unchanged from pre-dose levels (249 pg/mL versus 228 pg/mL pre-dose in Cohort 7).

Plasma cytokine levels after the first administration of the peptide composition were higher and more consistently elevated in Cohort 1 than Cohort 7, even though all subjects received the same dose (150 μg/mL) and peak plasma concentrations of the constituent peptides were similar. Among the cytokines and chemokines that were elevated in at least half of the peptide composition—treated subjects, the maximum elevation in plasma concentration from pre-dose levels during the 6 hours after the first dose of the peptide composition was always highest in Cohort 1 and lowest in Cohort 7. Further to the comparisons of IL-8, MCP-1, and IL-2 between Cohorts 1 and 7 described above, the maximal change from pre-dose concentrations for the following analytes was also greater in Cohort 1 than in Cohort 7:

IL-6: 214 g/mL in Cohort 1 versus 33 pg/mL in Cohort 7

IP-10: 6117 pg/mL in Cohort 1 versus 3218 pg/mL in Cohort 7

IL-10: 160 pg/mL in Cohort 1 versus 20 pg/mL in Cohort 7

Eotaxin: 1392 pg/mL in Cohort 1 versus 88 pg/mL in Cohort 7

TNF-α: 47 pg/mL in Cohort 1 versus 16 pg/mL in Cohort 7

Macrophage inflammatory protein (MIP)-1a: 50 pg/mL in Cohort 1 versus 5 pg/mLin

### Cohort 7

MIP-1β: 177 pg/mL in Cohort 1 versus 47 pg/mL in Cohort 7

Granulocyte-colony stimulating factor (G-CSF): 1828 pg/mL in Cohort 1 versus 81 pg/mL in Cohort 7

Furthermore, despite the fact that peak plasma concentrations of the constituent peptides were generally higher in Cohort 2, the fold-changes in plasma cytokine levels 6 hours after the first dose of the peptide composition were either similar or frequently higher in Cohort 1 than in Cohort 2. The pronounced differences in plasma cytokine responses between Cohorts 1 and 7 may be explained by reactivity to the peptide composition being boosted by prior oral gluten challenge in Cohort 1 subjects. However, a further explanation for the observed differences in circulating plasma cytokine levels between cohorts may be that cohorts were poorly matched for factors modifying responsiveness to the peptide composition.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
measuring a level of at least one circulating cytokine or chemokine in a subject that has or is suspected of having celiac disease, wherein the subject has been administered a first composition comprising at least one gluten peptide, and
assessing the likelihood the subject has celiac disease.

2. The method of claim 1, wherein the method further comprises obtaining a sample from the subject and the measuring is performed on the sample.

3. The method of claim 2, wherein the sample from the subject is obtained 1 hour to 6 hours after the subject has been administered the first composition.

4. The method of claim 2 or 3, where the sample from the subject is a plasma or serum sample.

5. The method of any one of claims 1 to 4, wherein the subject has been administered the first composition by injection.

6. The method of any one of claims 1 to 4, wherein the subject has been administered the first composition by oral administration.

7. The method of claim 6, wherein the first composition comprises a foodstuff that contains gluten.

8. The method of any one of claims 1 to 7, wherein the method further comprises administering the first composition to the subject prior to measuring the level of the at least one circulating cytokine or chemokine.

9. The method of any one of claims 1 to 8, wherein the at least one circulating cytokine or chemokine is MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 or sCD40L.

10. The method of claim 9, wherein the at least one circulating cytokine or chemokine is MCP-1, IL-8 or G-CSF.

11. The method of claim 9, wherein the at least one circulating cytokine or chemokine is selected from MCP-1, IL-10, IL-6, IL-8, IL-2 and G-CSF.

12. The method of claim 9, wherein the at least one circulating cytokine or chemokine is MCP-1.

13. The method of claim 9, wherein the at least one circulating cytokine or chemokine is IL-8.

14. The method of claim 9, wherein the at least one circulating cytokine or chemokine is IL-8 and MCP-1.

15. The method of claim 9, wherein the at least one circulating cytokine or chemokine is IL-2, IL-8 and MCP-1.

16. The method of any one of the claims 1 to 15, wherein an elevated level of the at least one circulating cytokine or chemokine compared to a control level of the at least one circulating cytokine or chemokine indicates that the subject has celiac disease, and the step of assessing comprises comparing the level of the at least one circulating cytokine or chemokine to a control level of the at least one circulating cytokine or chemokine.

17. The method of any one of claims 1 to 16, wherein the control level is a baseline level.

18. The method of claim 17, wherein the baseline level is a level of the at least one circulating cytokine or chemokine prior to administration of the first composition.

19. The method of any one of claims 1 to 18, wherein the method further comprises recording whether or not the subject has celiac disease based on the assessing.

20. The method of any one of claims 1 to 19, further comprising treating, suggesting a treatment, or giving information in regard to a treatment to the subject.

21. The method of claim 20, wherein the treating or treatment comprises administration of a second composition comprising a gluten peptide to the subject.

22. The method of any one of claims 1 to 21, wherein measuring the level of the at least one circulating cytokine or chemokine comprises an immuno-based assay.

23. The method of claim 22, wherein the immuno-based assay comprises an ELISA or a multiplex bead-based assay.

24. The method of any one of claims 1 to 23, wherein the first composition comprises at least one of:
(a) a first peptide comprising the amino acid sequence PFPQPELPY (SEQ ID NO: 1) and PQPELPYPQ (SEQ ID NO: 2);
(b) a second peptide comprising the amino acid sequence PFPQPEQPF (SEQ ID NO: 3) and PQPEQPFPW (SEQ ID NO: 4); and
(c) a third peptide comprising the amino acid sequence PIPEQPQPY (SEQ ID NO: 5).

25. The method of any one of claims 1 to 24, wherein the first composition comprises at least one of:
(a) a first peptide comprising the amino acid sequence PFPQPELPY (SEQ ID NO: 1) and PQPELPYPQ (SEQ ID NO: 2);
(b) a second peptide comprising the amino acid sequence PFPQPEQPF (SEQ ID NO: 3) and PQPEQPFPW (SEQ ID NO: 4); and
(c) a third peptide comprising the amino acid sequence PIPEQPQPY (SEQ ID NO: 5) and EQPIPEQPQ (SEQ ID NO: 106).

26. The method of claim 24 or 25, wherein the first composition comprises the first and second peptide, the first and third peptide, or the second and third peptide.

27. The method of claim 26, wherein the first composition comprises the first and second peptide.

28. The method of claim 24 or 25, wherein the first composition comprises the first, second, and third peptide.

29. The method of claim 28, wherein the first composition comprises:
10 micrograms of the first peptide and an equimolar amount of each of the second and third peptides;
15 micrograms of the first peptide and an equimolar amount of each of the second and third peptides;
20 micrograms of the first peptide and an equimolar amount of each of the second and third peptides; or
50 micrograms of the first peptide and an equimolar amount of each of the second and third peptides.

30. The method of claim 28 or 29, wherein the first composition is administered once to the subject.

31. The method of claim 21, wherein the second composition comprises at least one of:
(a) the first peptide comprising the amino acid sequence PFPQPELPY (SEQ ID NO: 1) and PQPELPYPQ (SEQ ID NO: 2);
(b) the second peptide comprising the amino acid sequence PFPQPEQPF (SEQ ID NO: 3) and PQPEQPFPW (SEQ ID NO: 4); and
(c) the third peptide comprising the amino acid sequence PIPEQPQPY (SEQ ID NO: 5).

32. The method of claim 21 or 31, wherein the second composition comprises at least one of:
(a) the first peptide comprising the amino acid sequence PFPQPELPY (SEQ ID NO: 1) and PQPELPYPQ (SEQ ID NO: 2);
(b) the second peptide comprising the amino acid sequence PFPQPEQPF (SEQ ID NO: 3) and PQPEQPFPW (SEQ ID NO: 4); and
(c) the third peptide comprising the amino acid sequence PIPEQPQPY (SEQ ID NO: 5) and EQPIPEQPQ (SEQ ID NO: 106).

33. The method of claim 31 or 32, wherein the second composition comprises the first and second peptide, the first and third peptide, or the second and third peptide.

34. The method of claim 33, wherein the second composition comprises the first and second peptide.

35. The method of claim 31 or 32, wherein the second composition comprises the first, second, and third peptide.

36. The method of any one claims 24 to 35, wherein the first peptide comprises LQPFPQPELPYPQPQ (SEQ ID NO: 6); the second peptide comprises QPFPQPEQPFPWQP (SEQ ID NO: 7); and the third peptide comprises PEQPIPEQPQPYPQQ (SEQ ID NO: 8).

37. The method of any one of claims 24 to 36, wherein the first, second and/or third peptides comprise an N-terminal acetyl group or pyroglutamate group, and/or a C terminal amide group.

38. The method of claim 37, wherein the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and the third peptide comprises EPEQPIPEQPQPYPQQ (SEQ ID NO: 11), wherein the N-terminal E is a pyroglutamate.

39. The method of any one of claims 1 to 38, wherein the subject is HLA-DQ2.5 positive.

40. The method of any one of claims 1 to 39, wherein the method further comprises measuring a T cell response to the first composition comprising the at least one gluten peptide.

41. The method of claim 40, wherein the measuring a T cell response comprises contacting a sample comprising a T cell from the subject with the first composition comprising the at least one gluten peptide and measuring the T cell response in the sample.

42. The method of claim 41, wherein the T cell response is measured by measuring a level of IFN-γ.

43. The method of claim 42, wherein measuring the level of IFN-γ comprises an immuno-based assay.

44. The method of claim 43, wherein the immuno-based assay comprises an ELISA or multiplex bead-based assay.

45. A kit comprising i) the first composition as defined in any one of claims 1 to 44, ii) a means for injecting the first composition, and iii) a binding partner for the at least one cytokine or chemokine as defined in any one of claims 1 to 44.

46. A kit comprising i) the first composition as defined in any one of claims 1 to 44 and ii) a binding partner for any one of the cytokines or chemokines as defined in any one of claims 1 to 44.

47. The kit of claim 46, wherein the binding partner is for MCP-1, IL-2, IL-8 or G-CSF.

48. The kit of claim 46 or 47, wherein the binding partner is for MCP-1.

49. The kit of claim 48, further comprising a binding partner for IL-2 or a binding partner for IL-8.

50. The kit of claim 49, further comprising a binding partner for IL-2 and a binding partner for IL-8.

51. The kit of any one of claims 46 to 50, further comprising a binding partner for IFN-γ.

52. The kit of any one of claims 46 to 50, further comprising the second composition as defined in any one of the preceding claims.

53. A method for assessing tolerance to a gluten peptide in a subject having Celiac disease, the method comprising:
(a) measuring in a subject having Celiac disease that has been administered a first composition comprising at least one gluten peptide a level of at least one circulating cytokine or chemokine; and
(b) assessing the tolerance of the subject to the at least one gluten peptide based on the measuring.

54. The method of claim 53, wherein the method further comprises obtaining a sample from the subject and the measuring is performed on the sample.

55. The method of claim 53 or 54, wherein the subject has been administered the first composition by injection or oral administration.

56. The method of claim 55, wherein the subject has been administered the first composition by injection.

57. The method of claim 55, wherein the subject has been administered the first composition by oral administration.

58. The method of claim 57, wherein the first composition comprises a foodstuff that contains gluten.

59. The method of any one of claims 53 to 58, wherein the method further comprises administering the first composition to the subject prior to the measuring.

60. The method of any one of claims 53 to 59, wherein the assessing comprises comparing the level of the at least one circulating cytokine or chemokine to a circulating cytokine or chemokine control level.

61. The method of any one of claims 53 to 60, wherein the method further comprises treating the subject prior to measuring and assessing.

62. The method of any one of claims 53 to 61, wherein the method further comprises treating the subject or suggesting a treatment to the subject based on the assessing.

63. The method of claim 62, wherein the treating comprises continuing with the treatment, or the suggesting comprises suggesting the subject continue with the treatment, based on the assessing.

64. The method of claim 62, wherein the treating comprises ceasing the treatment, or the suggesting comprises suggesting the subject cease the treatment, based on the assessing.

65. The method of claim 62, wherein the treating comprises administering a different or additional treatment, or the suggesting comprises suggesting the subject be treated with an additional or different treatment, based on the assessing.

66. The method of any one of claims 53 to 65, further comprising recording the level(s), the result(s) of the assessing and/or the treatment, or suggestion for treatment, based on the assessing.

67. The method of any one of claims 53 to 66, wherein the at least one circulating cytokine or chemokine is selected from MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 and sCD40L.

68. The method of any one of claims 53 to 67, wherein the at least one circulating cytokine or chemokine is selected from MCP-1, IL-10, IL-6, IL-8, IL-2 and G-CSF.

69. The method of claim 68, wherein the at least one circulating cytokine or chemokine is MCP-1.

70. The method of claim 67, wherein the at least one circulating cytokine or chemokine is IL-2, IL-8, IL-10, or MCP-1.

71. The method of claim 70, wherein the at least one circulating cytokine or chemokine is at least four circulating cytokines or chemokines comprising IL-2, IL-8, IL-10, and MCP-1.

72. The method of claim 70, wherein the at least one circulating cytokine or chemokine is at least three circulating cytokines or chemokines comprising IL-2, IL-8, and MCP-1.

73. The method of any one of claims 53 to 72, wherein measuring the level of the at least one circulating cytokine or chemokine comprises an immuno-based assay.

74. The method of claim 73, wherein the immuno-based assay comprises an ELISA or a multiplex bead-based assay.

75. The method of any one of claims 53 to 74, where the sample obtained from the subject is a plasma, serum, or urine sample.

76. The method of any one of claims 53 to 75, where the sample obtained from the subject is a plasma or serum sample.

77. The method of any one of claims 53 to 76, where the sample is obtained from the subject within 4-6 hours of administration of the first composition.

78. The method of any one of claims 53 to 77, wherein the composition comprises at least one of:
(a) a first peptide comprising the amino acid sequence PFPQPELPY (SEQ ID NO: 1) and PQPELPYPQ (SEQ ID NO: 2);
(b) a second peptide comprising the amino acid sequence PFPQPEQPF (SEQ ID NO: 3) and PQPEQPFPW (SEQ ID NO: 4); and
(c) a third peptide comprising the amino acid sequence PIPEQPQPY (SEQ ID NO: 5).

79. The method of any one of claims 53 to 78, wherein the composition comprises at least one of:
(a) a first peptide comprising the amino acid sequence PFPQPELPY (SEQ ID NO: 1) and PQPELPYPQ (SEQ ID NO: 2);
(b) a second peptide comprising the amino acid sequence PFPQPEQPF (SEQ ID NO: 3) and PQPEQPFPW (SEQ ID NO: 4); and
(c) a third peptide comprising the amino acid sequence PIPEQPQPY (SEQ ID NO: 5) and EQPIPEQPQ (SEQ ID NO: 106).

80. The method of claim 78 or 79, wherein the composition comprises the first and second peptide, the first and third peptide, or the second and third peptide.

81. The method of claim 78 or 79, wherein the composition comprises the first and second peptide.

82. The method of claim 78 or 79, wherein the composition comprises the first, second, and third peptide.

83. The method of claim 82, wherein the first composition comprises:
10 micrograms of the first peptide and an equimolar amount of each of the second and third peptides;
15 micrograms of the first peptide and an equimolar amount of each of the second and third peptides;
20 micrograms of the first peptide and an equimolar amount of each of the second and third peptides; or
50 micrograms of the first peptide and an equimolar amount of each of the second and third peptides.

84. The method of claim 82 or 83, wherein the first composition is administered once to the subject.

85. The method of any one claims 79 to 84, wherein the first peptide comprises LQPFPQPELPYPQPQ (SEQ ID NO: 6); the second peptide comprises QPFPQPEQPFPWQP (SEQ ID NO: 7); and the third peptide comprises PEQPIPEQPQPYPQQ (SEQ ID NO: 8).

86. The method of any one of claims 79 to 85, wherein the first, second and/or third peptides comprise an N-terminal acetyl group or pyroglutamate group, and/or a C terminal amide group.

87. The method of claim 86, wherein the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and the third peptide comprises EPEQPIPEQPQPYPQQ (SEQ ID NO: 11), wherein the N-terminal E is a pyroglutamate.

88. The method of any one of claims 53 to 87, further comprising orally administering or directing the subject to consume gluten prior to the measuring step.

89. The method of claim 88, wherein the subject is orally administered or directed to consume gluten for three days.

90. The method of claim 88 or 89, wherein the measuring step is performed six days after the last of the gluten is orally administered or consumed.

91. The method of any one of claims 53 to 90, wherein the method further comprises measuring a T cell response to the first composition comprising the at least one gluten peptide.

92. The method of claim 91, wherein the measuring a T cell response comprises contacting a sample comprising a T cell from the subject with the first composition comprising the at least one gluten peptide and measuring the T cell response in the sample.

93. The method of claim 92, wherein the T cell response is measured by measuring a level of IFN-γ.

94. The method of claim 93, wherein measuring the level of IFN-γ comprises an immuno-based assay.

95. The method of claim 94, wherein the immuno-based assay comprises an ELISA.

96. A method for assessing the efficacy of treatment of celiac disease, the method comprising:
(a) measuring in a subject that has been administered a first composition comprising at least one gluten peptide
(i) a level of at least one circulating cytokine or chemokine,
and/or
(ii) a level of at least one circulating T cell; and
(b) assessing the efficacy based on the measuring.

97. The method of claim 96, wherein the method further comprises (c) treating the subject, or suggesting a treatment to the subject, based on the assessing.

98. The method of claim 96 or 97, wherein the method further comprises obtaining a sample from the subject and the measuring is performed on the sample.

99. The method of any one of claims 96 to 98, wherein the subject has been administered the first composition by injection or oral administration.

100. The method of claim 99, wherein the subject has been administered the first composition by injection.

101. The method of any one of claims 53 to 100, wherein the method further comprises administering the first composition to the subject prior to the measuring.

102. The method of any one of claims 53 to 101, wherein the assessing comprises comparing the level of the at least one circulating cytokine or chemokine, and/or the level of at least one circulating T cell, to a circulating cytokine or chemokine control level, and/or a circulating T cell control level.

103. The method of any one of claims 53 to 102, wherein the treating comprises continuing with the treatment, or the suggesting comprises suggesting the subject continue with the treatment, based on the assessing.

104. The method of any one of claims 53 to 103, wherein the treating comprises ceasing the treatment, or the suggesting comprises suggesting the subject cease the treatment, based on the assessing.

105. The method of any one of claims 53 to 104, wherein the treating comprises administering a different or additional treatment, or the suggesting comprises suggesting the subject be treated with an additional or different treatment, based on the assessing.

106. The method of any one of claims 53 to 105 further comprising recording the level(s), the result(s) of the assessing and/or the treatment, or suggestion for treatment, based on the assessing.

107. The method of any one of claims 53 to 106, wherein the at least one circulating cytokine or chemokine is selected from MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 and sCD40L.

108. The method of claim 107, wherein the at least one circulating cytokine or chemokine is selected from MCP-1, IL-10, IL-6, IL-8, IL-2 and G-CSF.

109. The method of claim 108, wherein the at least one circulating cytokine or chemokine is MCP-1.

110. The method of claim 107, wherein the at least one circulating cytokine or chemokine is IL-2, IL-8, IL-10, or MCP-1.

111. The method of claim 107, wherein the at least one circulating cytokine or chemokine is at least four circulating cytokines or chemokines comprising IL-2, IL-8, IL-10, and MCP-1.

112. The method of claim 107, wherein the at least one circulating cytokine or chemokine is at least three circulating cytokines or chemokines comprising IL-2, IL-8, and MCP-1.

113. The method of any one of claims 53 to 112, wherein the at least one circulating T cell recognizes the at least one gluten peptide in the composition.

114. The method of any one of claims 53 to 113, wherein measuring the level of the at least one circulating cytokine or chemokine comprises an immuno-based assay.

115. The method of claim 114, wherein the immuno-based assay comprises an ELISA or a multiplex bead-based assay.

116. The method of any one of claims 53 to 115, wherein measuring the level of the at least one circulating T cell comprises a Major Histocompatibility Complex (MHC) tetramer assay.

117. The method of any one of claims 53 to 116, where the sample obtained from the subject is a plasma, serum, or urine sample.

118. The method of any one of claims 53 to 117, where the sample obtained from the subject is a plasma or serum sample.

119. The method of any one of claims 53 to 118, wherein the composition comprises at least one of:
(a) a first peptide comprising the amino acid sequence PFPQPELPY (SEQ ID NO: 1) and PQPELPYPQ (SEQ ID NO: 2);
(b) a second peptide comprising the amino acid sequence PFPQPEQPF (SEQ ID NO: 3) and PQPEQPFPW (SEQ ID NO: 4); and
(c) a third peptide comprising the amino acid sequence PIPEQPQPY (SEQ ID NO: 5).

120. The method of any one of claims 53 to 119, wherein the composition comprises at least one of:
(a) a first peptide comprising the amino acid sequence PFPQPELPY (SEQ ID NO: 1) and PQPELPYPQ (SEQ ID NO: 2);
(b) a second peptide comprising the amino acid sequence PFPQPEQPF (SEQ ID NO: 3) and PQPEQPFPW (SEQ ID NO: 4); and
(c) a third peptide comprising the amino acid sequence PIPEQPQPY (SEQ ID NO: 5) and EQPIPEQPQ (SEQ ID NO: 106).

121. The method of claim 119 or 120, wherein the composition comprises the first and second peptide, the first and third peptide, or the second and third peptide.

122. The method of claim 121, wherein the composition comprises the first and second peptide.

123. The method of claim 119 or 120, wherein the composition comprises the first, second, and third peptide.

124. The method of claim 123, wherein the first composition comprises:
10 micrograms of the first peptide and an equimolar amount of each of the second and third peptides;
15 micrograms of the first peptide and an equimolar amount of each of the second and third peptides;
20 micrograms of the first peptide and an equimolar amount of each of the second and third peptides; or
50 micrograms of the first peptide and an equimolar amount of each of the second and third peptides.

125. The method of claim 123 or 124, wherein the first composition is administered once to the subject.

126. The method of any one claims 119 to 125, wherein the first peptide comprises LQPFPQPELPYPQPQ (SEQ ID NO: 6); the second peptide comprises QPFPQPEQPFPWQP (SEQ ID NO: 7); and the third peptide comprises PEQPIPEQPQPYPQQ (SEQ ID NO: 8).

127. The method of any one of claims 119 to 126, wherein the first, second and/or third peptides comprise an N-terminal acetyl group or pyroglutamate group, and/or a C terminal amide group.

128. The method of claim 127, wherein the first peptide comprises ELQPFPQPELPYPQPQ (SEQ ID NO: 9), wherein the N-terminal E is a pyroglutamate; the second peptide comprises EQPFPQPEQPFPWQP (SEQ ID NO: 10), wherein the N-terminal E is a pyroglutamate; and the third peptide comprises EPEQPIPEQPQPYPQQ (SEQ ID NO: 11), wherein the N-terminal E is a pyroglutamate.

129. The method of any one of claims 53 to 128, further comprising orally administering or directing the subject to consume gluten prior to the measuring step.

130. The method of claim 129, wherein the subject is orally administered or directed to consume gluten for three days.

131. The method of claim 129 or 130, wherein the measuring step is performed six days after the last of the gluten is orally administered or consumed.

132. The method of any one of claims 53 to 131, wherein the method further comprises measuring a T cell response to the first composition comprising the at least one gluten peptide.

133. The method of claim 132, wherein the measuring a T cell response comprises contacting a sample comprising a T cell from the subject with the first composition comprising the at least one gluten peptide and measuring the T cell response in the sample.

134. The method of claim 133, wherein the T cell response is measured by measuring a level of IFN-γ.

135. The method of claim 134, wherein measuring the level of IFN-γ comprises an immuno-based assay.

136. The method of claim 135, wherein the immuno-based assay comprises an ELISA.

137. A kit comprising i) the first composition as defined in any one of claims 53 to 136, ii) a means for injecting the first composition, and iii) a binding partner for the at least one cytokine, chemokine, or T cell as defined in any one of claims 53 to 136.

138. A kit comprising i) the first composition as defined in any one of claims 53 to 136 and ii) a binding partner for MCP-1, IL-2 or IL-8.

139. The kit of claim 138, when the binding partner is for MCP-1, the kit further comprising a binding partner for IL-2 or a binding partner for IL-8.

140. The kit of claim 139, further comprising a binding partner for IL-2 and a binding partner for IL-8.

141. The kit of any one of claims 137 to 140, further comprising a binding partner for IFN-γ.


SEQUENCE LISTING (107 sequences)

SEQ ID NO: 1 - protein, 9 aa, Artificial Sequence
  Synthetic Polypeptide
PFPQPELPY

SEQ ID NO: 2 - protein, 9 aa, Artificial Sequence
  Synthetic Polypeptide
PQPELPYPQ

SEQ ID NO: 3 - protein, 9 aa, Artificial Sequence
  Synthetic Polypeptide
PFPQPEQPF

SEQ ID NO: 4 - protein, 9 aa, Artificial Sequence
  Synthetic Polypeptide
PQPEQPFPW

SEQ ID NO: 5 - protein, 9 aa, Artificial Sequence
  Synthetic Polypeptide
PIPEQPQPY

SEQ ID NO: 6 - protein, 15 aa, Artificial Sequence
  Synthetic Polypeptide
LQPFPQPELPYPQPQ

SEQ ID NO: 7 - protein, 14 aa, Artificial Sequence
  Synthetic Polypeptide
QPFPQPEQPFPWQP

SEQ ID NO: 8 - protein, 15 aa, Artificial Sequence
  Synthetic Polypeptide
PEQPIPEQPQPYPQQ

SEQ ID NO: 9 - protein, 16 aa, Artificial Sequence
  Synthetic Polypeptide
XLQPFPQPELPYPQPQ

SEQ ID NO: 10 - protein, 15 aa, Artificial Sequence
  Synthetic Polypeptide
XQPFPQPEQPFPWQP

SEQ ID NO: 11 - protein, 16 aa, Artificial Sequence
  Synthetic Polypeptide
XPEQPIPEQPQPYPQQ

SEQ ID NO: 12 - protein, 4 aa, Artificial Sequence
  Synthetic Polypeptide
PELP

SEQ ID NO: 13 - protein, 5 aa, Artificial Sequence
  Synthetic Polypeptide
PELPY

SEQ ID NO: 14 - protein, 9 aa, Artificial Sequence
  Synthetic Polypeptide
PYPQPELPY

SEQ ID NO: 15 - protein, 9 aa, Artificial Sequence
  Synthetic Polypeptide
PFPQPQLPY

SEQ ID NO: 16 - protein, 4 aa, Artificial Sequence
  Synthetic Polypeptide
PQLP

SEQ ID NO: 17 - protein, 5 aa, Artificial Sequence
  Synthetic Polypeptide
PQLPY

SEQ ID NO: 18 - protein, 9 aa, Artificial Sequence
  Synthetic Polypeptide
PYPQPQLPY

SEQ ID NO: 19 - protein, 9 aa, Artificial Sequence
  Synthetic Polypeptide
PFPQPQLPY

SEQ ID NO: 20 - protein, 9 aa, Artificial Sequence
  Synthetic Polypeptide
PQPQLPYPQ

SEQ ID NO: 21 - protein, 9 aa, Artificial Sequence
  Synthetic Polypeptide
PFPQPQQPF

SEQ ID NO: 22 - protein, 9 aa, Artificial Sequence
  Synthetic Polypeptide
PQPQQPFPW

SEQ ID NO: 23 - protein, 9 aa, Artificial Sequence
  Synthetic Polypeptide
PIPQQPQPY

SEQ ID NO: 24 - protein, 15 aa, Artificial Sequence
  Synthetic Polypeptide
LQPFPQPQLPYPQPQ

SEQ ID NO: 25 - protein, 14 aa, Artificial Sequence
  Synthetic Polypeptide
QPFPQPQQPFPWQP

SEQ ID NO: 26 - protein, 15 aa, Artificial Sequence
  Synthetic Polypeptide
PEQPIPQQPQPYPQQ

SEQ ID NO: 27 - protein, 9 aa, Artificial Sequence
  Synthetic Polypeptide
PFPQPDLPY

SEQ ID NO: 28 - protein, 9 aa, Artificial Sequence
  Synthetic Polypeptide
PQPELPYPQ

SEQ ID NO: 29 - protein, 9 aa, Artificial Sequence
  Synthetic Polypeptide
FRPEQPYPQ

SEQ ID NO: 30 - protein, 9 aa, Artificial Sequence
  Synthetic Polypeptide
PQQSFPEQQ

SEQ ID NO: 31 - protein, 9 aa, Artificial Sequence
  Synthetic Polypeptide
IQPEQPAQL

SEQ ID NO: 32 - protein, 9 aa, Artificial Sequence
  Synthetic Polypeptide
QQPEQPYPQ

SEQ ID NO: 33 - protein, 9 aa, Artificial Sequence
  Synthetic Polypeptide
SQPEQEFPQ

SEQ ID NO: 34 - protein, 9 aa, Artificial Sequence
  Synthetic Polypeptide
PQPEQEFPQ

SEQ ID NO: 35 - protein, 9 aa, Artificial Sequence
  Synthetic Polypeptide
QQPEQPFPQ

SEQ ID NO: 36 - protein, 9 aa, Artificial Sequence
  Synthetic Polypeptide
PQPEQPFCQ

SEQ ID NO: 37 - protein, 9 aa, Artificial Sequence
  Synthetic Polypeptide
QQPFPEQPQ

SEQ ID NO: 38 - protein, 9 aa, Artificial Sequence
  Synthetic Polypeptide
PFPQPEQPF

SEQ ID NO: 39 - protein, 9 aa, Artificial Sequence
  Synthetic Polypeptide
PQPEQPFPW

SEQ ID NO: 40 - protein, 9 aa, Artificial Sequence
  Synthetic Polypeptide
PFSEQEQPV

SEQ ID NO: 41 - protein, 9 aa, Artificial Sequence
  Synthetic Polypeptide
FSQQQESPF

SEQ ID NO: 42 - protein, 9 aa, Artificial Sequence
  Synthetic Polypeptide
PFPQPEQPF

SEQ ID NO: 43 - protein, 9 aa, Artificial Sequence
  Synthetic Polypeptide
PQPEQPFPQ

SEQ ID NO: 44 - protein, 9 aa, Artificial Sequence
  Synthetic Polypeptide
PIPEQPQPY

SEQ ID NO: 45 - protein, 9 aa, Artificial Sequence
  Synthetic Polypeptide
PFPQPEQPF

SEQ ID NO: 46 - protein, 9 aa, Artificial Sequence
  Synthetic Polypeptide
PQPEQPFPQ

SEQ ID NO: 47 - protein, 9 aa, Artificial Sequence
  Synthetic Polypeptide
PYPEQEEPF

SEQ ID NO: 48 - protein, 9 aa, Artificial Sequence
  Synthetic Polypeptide
PYPEQEQPF

SEQ ID NO: 49 - protein, 9 aa, Artificial Sequence
  Synthetic Polypeptide
PFSEQEQPV

SEQ ID NO: 50 - protein, 9 aa, Artificial Sequence
  Synthetic Polypeptide
EGSFQPSQE

SEQ ID NO: 51 - protein, 9 aa, Artificial Sequence
  Synthetic Polypeptide
EQPQQPFPQ

SEQ ID NO: 52 - protein, 9 aa, Artificial Sequence
  Synthetic Polypeptide
EQPQQPYPE

SEQ ID NO: 53 - protein, 10 aa, Artificial Sequence
  Synthetic Polypeptide
QQGYYPTSPQ

SEQ ID NO: 54 - protein, 9 aa, Artificial Sequence
  Synthetic Polypeptide
EGSFQPSQE

SEQ ID NO: 55 - protein, 9 aa, Artificial Sequence
  Synthetic Polypeptide
PQQSFPEQE

SEQ ID NO: 56 - protein, 9 aa, Artificial Sequence
  Synthetic Polypeptide
QGYYPTSPQ

SEQ ID NO: 57 - protein, 9 aa, Artificial Sequence
  Synthetic Polypeptide
PQPQLPYPQ

SEQ ID NO: 58 - protein, 9 aa, Artificial Sequence
  Synthetic Polypeptide
FRPQQPYPQ

SEQ ID NO: 59 - protein, 9 aa, Artificial Sequence
  Synthetic Polypeptide
PQQSFPQQQ

SEQ ID NO: 60 - protein, 9 aa, Artificial Sequence
  Synthetic Polypeptide
IQPQQPAQL

SEQ ID NO: 61 - protein, 9 aa, Artificial Sequence
  Synthetic Polypeptide
QQPQQPYPQ

SEQ ID NO: 62 - protein, 9 aa, Artificial Sequence
  Synthetic Polypeptide
SQPQQQFPQ

SEQ ID NO: 63 - protein, 9 aa, Artificial Sequence
  Synthetic Polypeptide
PQPQQQFPQ

SEQ ID NO: 64 - protein, 9 aa, Artificial Sequence
  Synthetic Polypeptide
QQPQQPFPQ

SEQ ID NO: 65 - protein, 9 aa, Artificial Sequence
  Synthetic Polypeptide
PQPQQPFCQ

SEQ ID NO: 66 - protein, 9 aa, Artificial Sequence
  Synthetic Polypeptide
QQPFPQQPQ

SEQ ID NO: 67 - protein, 9 aa, Artificial Sequence
  Synthetic Polypeptide
PFPQPQQPF

SEQ ID NO: 68 - protein, 9 aa, Artificial Sequence
  Synthetic Polypeptide
PQPQQPFPW

SEQ ID NO: 69 - protein, 9 aa, Artificial Sequence
  Synthetic Polypeptide
PFSQQQQPV

SEQ ID NO: 70 - protein, 9 aa, Artificial Sequence
  Synthetic Polypeptide
FSQQQQSPF

SEQ ID NO: 71 - protein, 9 aa, Artificial Sequence
  Synthetic Polypeptide
PFPQPQQPF

SEQ ID NO: 72 - protein, 9 aa, Artificial Sequence
  Synthetic Polypeptide
PQPQQPFPQ

SEQ ID NO: 73 - protein, 9 aa, Artificial Sequence
  Synthetic Polypeptide
PIPQQPQPY

SEQ ID NO: 74 - protein, 9 aa, Artificial Sequence
  Synthetic Polypeptide
PFPQPQQPF

SEQ ID NO: 75 - protein, 9 aa, Artificial Sequence
  Synthetic Polypeptide
PQPQQPFPQ

SEQ ID NO: 76 - protein, 9 aa, Artificial Sequence
  Synthetic Polypeptide
PYPEQQEPF

SEQ ID NO: 77 - protein, 9 aa, Artificial Sequence
  Synthetic Polypeptide
PYPEQQQPF

SEQ ID NO: 78 - protein, 9 aa, Artificial Sequence
  Synthetic Polypeptide
PFSQQQQPV

SEQ ID NO: 79 - protein, 9 aa, Artificial Sequence
  Synthetic Polypeptide
QGSFQPSQQ

SEQ ID NO: 80 - protein, 9 aa, Artificial Sequence
  Synthetic Polypeptide
QQPQQPFPQ

SEQ ID NO: 81 - protein, 9 aa, Artificial Sequence
  Synthetic Polypeptide
QQPQQPYPQ

SEQ ID NO: 82 - protein, 10 aa, Artificial Sequence
  Synthetic Polypeptide
QQGYYPTSPQ

SEQ ID NO: 83 - protein, 9 aa, Artificial Sequence
  Synthetic Polypeptide
QGSFQPSQQ

SEQ ID NO: 84 - protein, 9 aa, Artificial Sequence
  Synthetic Polypeptide
PQQSFPQQQ

SEQ ID NO: 85 - protein, 9 aa, Artificial Sequence
  Synthetic Polypeptide
QGYYPTSPQ

SEQ ID NO: 86 - protein, 15 aa, Artificial Sequence
  Synthetic Polypeptide
LQPFPQPELPYPQPQ

SEQ ID NO: 87 - protein, 14 aa, Artificial Sequence
  Synthetic Polypeptide
QPFPQPQQPFPWQP

SEQ ID NO: 88 - protein, 15 aa, Artificial Sequence
  Synthetic Polypeptide
PQQPIPQQPQPYPQQ

SEQ ID NO: 89 - protein, 7 aa, Artificial Sequence
  Synthetic Polypeptide
QPELPYP

SEQ ID NO: 90 - protein, 7 aa, Artificial Sequence
  Synthetic Polypeptide
PQPELPY

SEQ ID NO: 91 - protein, 7 aa, Artificial Sequence
  Synthetic Polypeptide
FPQPELP

SEQ ID NO: 92 - protein, 7 aa, Artificial Sequence
  Synthetic Polypeptide
PELPYPQ

SEQ ID NO: 93 - protein, 9 aa, Artificial Sequence
  Synthetic Polypeptide
FPQPELPYP

SEQ ID NO: 94 - protein, 9 aa, Artificial Sequence
  Synthetic Polypeptide
PQPDLPYPQ

SEQ ID NO: 95 - protein, 9 aa, Artificial Sequence
  Synthetic Polypeptide
PFPQPDQPF

SEQ ID NO: 96 - protein, 9 aa, Artificial Sequence
  Synthetic Polypeptide
PQPDQPFPW

SEQ ID NO: 97 - protein, 9 aa, Artificial Sequence
  Synthetic Polypeptide
PIPDQPQPY

SEQ ID NO: 98 - protein, 15 aa, Artificial Sequence
  Synthetic Polypeptide
LQPFPQPDLPYPQPQ

SEQ ID NO: 99 - protein, 14 aa, Artificial Sequence
  Synthetic Polypeptide
QPFPQPDQPFPWQP

SEQ ID NO: 100 - protein, 15 aa, Artificial Sequence
  Synthetic Polypeptide
PQQPIPDQPQPYPQQ

SEQ ID NO: 101 - protein, 7 aa, Artificial Sequence
  Synthetic Polypeptide
QPQLPYP

SEQ ID NO: 102 - protein, 7 aa, Artificial Sequence
  Synthetic Polypeptide
PQPQLPY

SEQ ID NO: 103 - protein, 7 aa, Artificial Sequence
  Synthetic Polypeptide
FPQPQLP

SEQ ID NO: 104 - protein, 7 aa, Artificial Sequence
  Synthetic Polypeptide
PQLPYPQ

SEQ ID NO: 105 - protein, 9 aa, Artificial Sequence
  Synthetic Polypeptide
FPQPQLPYP

SEQ ID NO: 106 - protein, 9 aa, Artificial Sequence
  Synthetic Polypeptide
EQPIPEQPQ

SEQ ID NO: 107 - protein, 9 aa, Artificial Sequence
  Synthetic Polypeptide
EQPIPQQPQ